(19) United States
(12) Patent Application Publication
(10) Pub. No.: US 2004/0005612 A1
(43) Pub. Date: Jan. 8, 2004

(54) Endometrial genes in endometrial disorders

(76) Inventors: Linda C. Giudice, Los Altos Hills, CA (US); Lee C. Kao, Foster City, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/437,733

(22) Filed: May 13, 2003

Related U.S. Application Data
(60) Provisional application No. 60/380,689, filed on May 14, 2002.

(51) Int. Cl. C12Q 1/68

(52) U.S. Cl. 435/6

(57) ABSTRACT

Genetic sequences are identified with expression levels that are upregulated or downregulated in human endometrium during the window of implantation. The endometrial signature of genes during the window of implantation provides diagnostic screening tests for patients with infertility and endometrial disorders, and endometriosis; and for targeted drug discovery for treating implantation-based infertility, other endometrial disorders, and endometrial-based contraception.

### BACKGROUND OF THE INVENTION

Implantation in humans involves complex interactions between the embryo and the maternal endometrium. Histologic examination of early human pregnancies reveals distinct patterns of blastocyst attachment to the endometrial surface and the underlying stroma, supporting a model of implantation in humans in which the embryo apposes and attaches to the endometrial epithelium, traverses adjacent cells of the epithelial lining, and invades into the endometrial stroma. The endometrium is receptive to embryonic implantation during a defined “window” that is temporally and spatially restricted.

The implantation process begins with attachment of the embryo to the endometrial epithelium, intrusion through the epithelium and then invasion into the decidualizing stromal compartment, eventually resulting in anchoring of the conceptus and establishment of the fetal placenta and blood supply. Molecular definition of the window of implantation in human endometrium is beginning to be understood, and several molecular “markers” of the window and of uterine receptivity to embryonic implantation have been identified.

Temporal definition of the window of implantation in human endometrium derives from several sources. Early studies suggest that the window resides in the mid-secretory phase, because embryos identified in secretory phase hysterectomy specimens were all free-floating before day 20 of the cycle and were all attached when specimens were obtained after day 20. In addition, the temporal and spatial appearance of epithelial dome-like structures (“pinopodes”) support a receptive phase of embryonic implantation, since they appear on cycle days 20-24, correlate with implantation sites, and are believed to participate in attachment of the embryo to the epithelium. A recent report demonstrates a high success (84%) of continuing pregnancy for embryos that implant between cycle days 22-24 (post-ovulatory day 8-10), compared to 18% when implantation occurred 11 days or more after ovulation (Wilcox et al. (1999) N Engl J Med 340:1776-1779). Together these data suggest that the window of implantation in humans spans cycle days 20-24 and involves the epithelium and subsequently underlying stroma.

Molecular definition of the window of implantation in human endometrium has been more difficult to define and derives primarily from animal models and clinical specimens, see Lessey (2000) Human Reprod 15:39-50. These studies have revealed a limited number of potential molecular “markers” of the implantation window and of uterine receptivity to embryonic implantation.

Animal models of homologous recombination and gene “knockouts” that demonstrate an implantation-based infertility phenotype provide important insight into potential markers for uterine receptivity and participants in the molecular mechanisms occurring during embryonic implantation into the maternal endometrium. By translation from such models and building upon a literature of known expressed genes and proteins and uniquely expressed secretory proteins in human endometrium, the expression of several molecules has been found to be specifically and temporally expressed within and framing the window of implantation in humans (Paria et al. (2000) Semin Cell Dev Biol 11:67-76), suggesting their functionality in the implantation process.

The molecular dialogue that occurs between the endometrium and the implanting conceptus involves cell-cell and cell-extracellular matrix interactions, mediated by lectins, integrins, matrix degrading enzymes and their inhibitors, and a variety of growth factors and cytokines, their receptors and modulatory proteins. Of note are molecules that participate in attachment of an embryo to the maternal endometrial epithelium, including carbohydrate epitopes (e.g, H-type 1 antigen), heparan sulfate proteoglycan, mucins, integrins (especially αᵥβ₃, α₄β₄), and the trophin-bystin/tastin complex. Molecules that participate in embryonic attachment to the epithelium and subsequent signaling between epithelium and stroma have been deduced from “knockout” studies of a given gene in mice that result in absence of embryonic attachment to the epithelium and loss of decidualization of the stroma. These molecules include leukemia inhibitor factor, the homeobox genes, HoxA-10 and HoxA-11, and cyclooxygenase 2 (COX-2).

Endometriosis is an estrogen-dependent, benign gynecologic disorder affecting about 10 to 15% of women of reproductive age. It is characterized by endometrial tissue found outside of the uterus (primarily in the pelvic cavity) and is associated with pelvic pain and infertility. A recent meta-analysis of assisted reproductive outcomes revealed that women with endometriosis and infertility who undergo in vitro fertilization and embryo transfer (IVF-ET) have pregnancy rates that are about 50% of women who undergo IVF-ET for tubal factor infertility. Abnormalities in the endometrium resulting in failure of embryonic implantation are believed largely to account for the lower pregnancy rates in women with endometriosis. However, since the pathogenesis of endometriosis per se is uncertain, the basis of implantation failure in women with endometriosis has been difficult to define.

In the pre-genomic era a “one-by-one” approach has been useful to reveal select candidates for uterine receptivity or to investigate endometrial abnormalities in women with or without endometriosis during the implantation window or at other times of the cycle. Recently, discovery-based genome-wide microarray comparisons have been used to broadly investigate various systems from yeast to cancers. Methods of high throughput analysis of gene expression are of interest to address this issue.

Literature

Wilcox et al. (1999) N Engl J Med 340:1776-1779; Lessey (2000) Human Reprod 15:39-50; Paria et al. (2000) Semin Cell Dev Biol 11:67-76; Giudice et al. (1998) J. Reprod. Med. 43(3 Suppl):252-262; Barnhart et al. (2002) Fertil. Steril. 77(6):1148-1155; Giudice et al. (2002) Ann. N.Y. Acad. Sci. 955:252-264; Lessey et al. (1994) Fertil. Steril. 62(3):497-506; Bhatt et al. (1991) Proc. Natl. Acad. Sci. U.S.A. 88(24):11408-11412; Kothapalli et al. (1997) J. Clin. Invest. 99(10):2342-2350; Noble et al. (1996) J. Clin. Endocrinol. Metab. 81(1):174-179; Zeitoun et al. (1998) J. Clin. Endocrinol. Metab. 83(12):4474-4480; Bruner-Tran et al. (2002) J. Clin. Endocrinol. Metab. 87(10):4782-4791; Kao et al. (2002) Endocrinology. 143(6):2119-2138; Carson et al. (2002) Mol. Hum. Reprod. 8(9):871-879; Arici et al. (1996) Fertil. Steril. 65(3):603-607; Valdes et al. (2001) Endocrine. 16(3):207-215; Nisolle et al. (1994) Fertil. Steril. 62(4):751-759; Attia et al. (2000) J. Clin. Endocrinol. Metab. 85(8):2897-2902; Okada et al. (2000) Mol. Hum. Reprod. 6(1):75-80; Kitaya et al. (2000) Biology of Reproduction. 63(3):683-687; Dunn et al. (2002) J. Clin. Endocrinol. Metab. 87(4): 1898-1901;

### SUMMARY OF THE INVENTION

Genetic sequences are identified with expression levels that are upregulated or downregulated in human endometrium during the window of implantation and associated with endometrial abnormalities. The data provide an expression signature of endometrial genes during the window of implantation that provides insight into the pathogenesis of implantation failure in women with endometriosis and a unique opportunity to design diagnostic tests for endometriosis and targeted drug discovery for endometriosis-based implantation failure.

These genes, gene families, and signaling pathways are provided that are candidates for uterine receptivity, and allow definition of molecular mechanisms underlying the process of human implantation. The endometrial signature of genes during the window of implantation provides diagnostic screening tests for patients with infertility and endometrial disorders, including endometriosis, and for targeted drug discovery for treating implantation-based infertility, other endometrial disorders, endometriosis, and endometrial-based contraception.

### BRIEF DESCRIPTION OF THE DRAWINGS

FIG. 1 depicts validation of selected genes >2-fold up- or down-regulated during the window of implantation in human endometrium by RT-PCR.

FIG. 2 depicts Northern analysis demonstrating up-regulation of Dkk-1, IGFBP-1, GABA_{A} R π subunit, glycodelin, and down-regulation of PGRMC-1, matrilysin and FrpHE in the secretory phase (implantation window, lane c), compared to the proliferative phase (lane b).

FIGS. 3A-B depict expression of selected genes in cultured human endometrial epithelial (Panel A) and stromal (Panel B) cells by RT-PCR.

FIG. 4 depicts equal cycle RT-PCR of selected genes up-regulated in eutopic human endometrium during the window of implantation, from women without (N) and with (D) endometriosis.

FIG. 5 depicts equal cycle RT-PCR of selected genes down-regulated in eutopic human endometrium during the window of implantation, from women without (N) and with (D) endometriosis.

FIGS. 6A-C depict Northern blot analyses demonstrating: (A) up-regulation of collagen alpha-2 type 1, (B) down-regulation of GlcNAc, glycodelin, integrin 2 α subunit and B61, in eutopic human endometrium during the window of implantation, from women without (a) or with (b) endometriosis.

### DETAILED DESCRIPTION OF THE EMBODIMENTS

Methods and compositions are provided for the diagnosis and treatment of infertility and endometrial disorders, including endometriosis. The invention is based, in part, on the evaluation of the expression and role of genes that are differentially expressed in endometrial tissue during the window of implantation. Endometrial tissue samples for expression analysis were taken at varying time points and analyzed for differential expression of genes. Identification of these genes permits the definition of physiological pathways, and the identification of targets in pathways that are useful both diagnostically and therapeutically.

The data presented herein provides an endometrial database of genes expressed during the window of implantation. Using microarray technology, global changes in gene expression in human endometrium are defined, and are extrapolated to defining the genetic profiles during the proliferative phase, peri-ovulatory phase, and during the late secretory phase in the absence of implantation and in preparation for menstrual desquamation. Global changes in gene expression can be determined in disorders of the endometrium, including implantation-related infertility (as in women with endometriosis), evaluation of the endometrium for normalcy in women with hyperandrogenic disorders, in normovulatory women in response to therapeutics in which the endometrium is targeted (or as a side effect of other therapies), as well as endometrial hyperplasia and endometrial cancers. Candidate genes are identified for the diagnosis of patients with infertility and for targeted drug discovery for enhancing (or inhibiting) implantation for infertility treatment (or contraception).

The identification of differentially expressed endometrial genes provides diagnostic and prognostic methods, which detect the occurrence of an endometrial disorder, or assess an individual's susceptibility to such disease. Therapeutic and prophylactic treatment methods for individuals suffering, or at risk of an endometrial disorder, involve administering either a therapeutic or prophylactic amount of an agent that modulates the activity of endometrial genes. Agents of interest include purified forms of the encoded protein, agents that stimulate expression or synthesis of such gene products, agents that block activity of such gene products or that down regulates the expression of such genes, or a nucleic acid, including coding sequences of endometrial genes or anti-sense or RNAi sequences corresponding to these genes.

Screening methods generally involve conducting various types of assays to identify agents that modulate the expression or activity of an endometrial target protein. Such screening methods can initially involve screens to identify compounds that can bind to the protein. Certain assays are designed to measure more clearly the effect that different agents have on gene product activities or expression levels. Lead compounds identified during these screens can serve as the basis for the synthesis of more active analogs. Lead compounds and/or active analogs generated therefrom can be formulated into pharmaceutical compositions effective in treating endometrial disorders and conditions.

In order to identify endometrial target genes, tissue was taken at defined time points during menstrual cycle. RNA was isolated from one or more such tissues. Differentially expressed genes were detected by comparing the pattern of gene expression. Once a particular gene was identified, its expression pattern was further characterized by DNA sequencing. Differential expression and expression patterns of genes may be confirmed by in situ hybridization or reverse transcription-polymerase chain reaction (RT-PCR) on tissue generated from normal samples, culture models, diseased tissue, etc.

“Differential expression” as used herein refers to both quantitative as well as qualitative differences in the genes' temporal and/or tissue expression patterns. Thus, a differentially expressed gene may have its expression activated or completely inactivated in normal versus endometrial disease conditions, or under control versus experimental conditions. Such a qualitatively regulated gene will exhibit an expression pattern within a given tissue or cell type that is detectable in either control or subjects with endometriosis, but is not detectable in both; or that is differentially expressed in subjects with endometriosis during the window of implantation. Detectable, as used herein, refers to an RNA expression pattern that is detectable via the standard techniques of differential display, reverse transcriptase-(RT-) PCR and/or Northern analyses, which are well known to those of skill in the art. Generally, differential expression means that there is at least a 20% change, and in other instances at least a 2-, 3-, 5- or 10-fold difference between disease and control tissue expression. The difference usually is one that is statistically significant, meaning that the probability of the difference occurring by chance (the P-value) is less than some predetermined level (e.g., 0.05). Usually the confidence level P is <0.05, more typically <0.01, and in other instances, <0.001.

Alternatively, a differentially expressed gene may have its expression modulated, i.e., quantitatively increased or decreased, in normal versus disease states, or under control versus experimental conditions. The difference in expression need only be large enough to be visualized via standard detection techniques as described above.

Once a sequence has been identified as differentially expressed, the sequence can be subjected to a functional validation process to determine whether the gene plays a role in disease, implantation, etc. Such candidate genes can potentially be correlated with a wide variety of cellular states or activities. The term “functional validation” as used herein refers to a process whereby one determines whether modulation of expression of a candidate gene or set of such genes causes a detectable change in a cellular activity or cellular state for a reference cell, which cell can be a population of cells such as a tissue or an entire organism. The detectable change or alteration that is detected can be any activity carried out by the reference cell. Specific examples of activities or states in which alterations can be detected include, but are not limited to, phenotypic changes (e.g., cell morphology, cell proliferation, cell viability and cell death); cells acquiring resistance to a prior sensitivity or acquiring a sensitivity which previously did not exist; protein/protein interactions; cell movement; intracellular or intercellular signaling; cell/cell interactions; cell activation; release of cellular components (e.g., hormones, chemokines and the like); and metabolic or catabolic reactions.

The identity of endometrial target genes is set forth in Tables 2 and 5, and Tables 3 and 6, for upregulated sequences and downregulated sequences, respectively. Nucleic acids comprising these sequences find use in diagnostic and prognostic methods, for the recombinant production of the encoded polypeptide, and the like. The nucleic acids of the invention include nucleic acids having a high degree of sequence similarity or sequence identity to the identified sequences. Sequence identity can be determined by hybridization under stringent conditions, for example, at 50° C. or higher and 0.1×SSC (9 mM NaCl/0.9 mM Na citrate). Hybridization methods and conditions are well known in the art, see, e.g., U.S. Pat. No. 5,707,829. Nucleic acids may also be substantially identical to the provided nucleic acid sequences, e.g. allelic variants, genetically altered versions of the gene, etc. Further specific guidance regarding the preparation of nucleic acids is provided by Fleury et al. (1997) Nature Genetics 15:269-272; Tartaglia et al., PCT Publication No. WO 96/05861; and Chen et al., PCT Publication No. WO 00/06087, each of which is incorporated herein in its entirety.

The endometrial target sequences may be obtained using various methods well known to those skilled in the art, including but not limited to the use of appropriate probes to detect the gene within an appropriate cDNA or genomic DNA library, antibody screening of expression libraries to detect cloned DNA fragments with shared structural features, direct chemical synthesis, and amplification protocols. Cloning methods are described in Berger and Kimmel, Guide to Molecular Cloning Techniques, Methods in Enzymology, 152, Academic Press, Inc. San Diego, Calif.; Sambrook, et al. (1989) Molecular Cloning—A Laboratory Manual (2nd ed) Vols. 1-3, Cold Spring Harbor Laboratory, Cold Spring Harbor Press, NY; and Current Protocols (1994), a joint venture between Greene Publishing Associates, Inc. and John Wiley and Sons, Inc.

Sequences obtained from partial clones can be used to obtain the entire coding region by using the rapid amplification of cDNA ends (RACE) method (Chenchik et al (1995) CLONTECHniques (X) 1: 5-8). Oligonucleotides can be designed based on the sequence obtained from the partial clone that can amplify a reverse transcribed mRNA encoding the entire coding sequence. Alternatively, probes can be used to screen cDNA libraries prepared from an appropriate cell or cell line in which the gene is transcribed. Once the target nucleic acid is identified, it can be isolated and cloned using well-known amplification techniques. Such techniques include the polymerase chain reaction (PCR) the ligase chain reaction (LCR), Qβ-replicase amplification, the self-sustained sequence replication system (SSR) and the transcription based amplification system (TAS). Such methods include, those described, for example, in U.S. Pat. No. 4,683,202 to Mullis et al.; PCR Protocols A Guide to Methods and Applications (Innis et al. eds) Academic Press Inc. San Diego, Calif. (1990); Kwoh et al. (1989) Proc. Natl. Acad. Sci. USA 86: 1173; Guatelli et al. (1990) Proc. Natl. Acad. Sci. USA 87: 1874; Lomell et al. (1989) J. Clin. Chem. 35: 1826; Landegren et al. (1988) Science 241: 1077-1080; Van Brunt (1990) Biotechnology 8: 291-294; Wu and Wallace (1989) Gene 4: 560; and Barringer et al. (1990) Gene 89: 117.

As an alternative to cloning a nucleic acid, a suitable nucleic acid can be chemically synthesized. Direct chemical synthesis methods include, for example, the phosphotriester method of Narang et al. (1979) Meth. Enzymol. 68: 90-99; the phosphodiester method of Brown et al. (1979) Meth. Enzymol. 68: 109-151; the diethylphosphoramidite method of Beaucage et al. (1981) Tetra. Lett., 22: 1859-1862; and the solid support method of U.S. Pat. No. 4,458,066. Chemical synthesis produces a single stranded oligonucleotide. This can be converted into double stranded DNA by hybridization with a complementary sequence, or by polymerization with a DNA polymerase using the single strand as a template. While chemical synthesis of DNA is often limited to sequences of about 100 bases, longer sequences can be obtained by the ligation of shorter sequences. Alternatively, subsequences may be cloned and the appropriate subsequences cleaved using appropriate restriction enzymes.

Nucleic acids used in the present methods can be cDNAs or genomic DNAs, as well as fragments thereof. The term “cDNA” as used herein is intended to include all nucleic acids that share the arrangement of sequence elements found in native mature mRNA species, where sequence elements are exons and 3′ and 5′ non-coding regions. Normally mRNA species have contiguous exons, with the intervening introns, when present, being removed by nuclear RNA splicing, to create a continuous open reading frame encoding a polypeptide of the invention.

A genomic sequence of interest comprises the nucleic acid present between the initiation codon and the stop codon, as defined in the listed sequences, including all of the introns that are normally present in a native chromosome. It can further include the 3′ and 5′ untranslated regions found in the mature mRNA. It can further include specific transcriptional and translational regulatory sequences, such as promoters, enhancers, etc., including about 1 kb, but possibly more, of flanking genomic DNA at either the 5′ or 3′ end of the transcribed region. The genomic DNA flanking the coding region, either 3′ or 5′, or internal regulatory sequences as sometimes found in introns, contains sequences required for proper tissue, stage-specific, or disease-state specific expression, and are useful for investigating the up-regulation of expression in endometrial cells.

Probes specific to an endometrial target gene is preferably at least about 18 nt, 25 nt, 50 nt or more of the corresponding contiguous sequence of one of the sequences identified in Table 2, Table 3, Table 5, Table 6, and are usually less than about 500 bp in length. Preferably, probes are designed based on a contiguous sequence that remains unmasked following application of a masking program for masking low complexity, e.g. BLASTX. Double or single stranded fragments can be obtained from the DNA sequence by chemically synthesizing oligonucleotides in accordance with conventional methods, by restriction enzyme digestion, by PCR amplification, etc. The probes can be labeled, for example, with a radioactive, biotinylated, or fluorescent tag.

The nucleic acids of the subject invention are isolated and obtained in substantial purity, generally as other than an intact chromosome. Usually, the nucleic acids, either as DNA or RNA, will be obtained substantially free of other naturally-occurring nucleic acid sequences, generally being at least about 50%, usually at least about 90% pure and are typically “recombinant,” e.g., flanked by one or more nucleotides with which it is not normally associated on a naturally occurring chromosome.

The nucleic acids of the invention can be provided as a linear molecule or within a circular molecule, and can be provided within autonomously replicating molecules (vectors) or within molecules without replication sequences. Expression of the nucleic acids can be regulated by their own or by other regulatory sequences known in the art. The nucleic acids of the invention can be introduced into suitable host cells using a variety of techniques available in the art, such as transferrin polycation-mediated DNA transfer, transfection with naked or encapsulated nucleic acids, liposome-mediated DNA transfer, intracellular transportation of DNA-coated latex beads, protoplast fusion, viral infection, electroporation, gene gun, calcium phosphate-mediated transfection, and the like.

For use in amplification reactions, such as PCR, a pair of primers will be used. The exact composition of the primer sequences is not critical to the invention, but for most applications the primers will hybridize to the subject sequence under stringent conditions, as known in the art. It is preferable to choose a pair of primers that will generate an amplification product of at least about 50 nt, preferably at least about 100 nt. Algorithms for the selection of primer sequences are generally known, and are available in commercial software packages. Amplification primers hybridize to complementary strands of DNA, and will prime towards each other. For hybridization probes, it may be desirable to use nucleic acid analogs, in order to improve the stability and binding affinity. The term “nucleic acid” shall be understood to encompass such analogs.

Polypeptides

Endometrial target polypeptides are of interest for screening methods, as reagents to raise antibodies, as therapeutics, and the like. Such polypeptides can be produced through isolation from natural sources, recombinant methods and chemical synthesis. In addition, functionally equivalent polypeptides may find use, where the equivalent polypeptide may contain deletions, additions or substitutions of amino acid residues that result in a silent change, thus producing a functionally equivalent differentially expressed on pathway gene product. Amino acid substitutions may be made on the basis of similarity in polarity, charge, solubility, hydrophobicity, hydrophilicity, and/or the amphipathic nature of the residues involved. “Functionally equivalent”, as used herein, refers to a protein capable of exhibiting a substantially similar in vivo activity as the starting polypeptide.

The polypeptides may be produced by recombinant DNA technology using techniques well known in the art. Methods which are well known to those skilled in the art can be used to construct expression vectors containing coding sequences and appropriate transcriptional/translational control signals. These methods include, for example, in vitro recombinant DNA techniques, synthetic techniques and in vivo recombination/genetic recombination. Alternatively, RNA capable of encoding the polypeptides of interest may be chemically synthesized.

Typically, the coding sequence is placed under the control of a promoter that is functional in the desired host cell to produce relatively large quantities of the gene product. An extremely wide variety of promoters are well known, and can be used in the expression vectors of the invention, depending on the particular application. Ordinarily, the promoter selected depends upon the cell in which the promoter is to be active. Other expression control sequences such as ribosome binding sites, transcription termination sites and the like are also optionally included. Constructs that include one or more of this control sequences are termed “expression cassettes.” Expression can be achieved in prokaryotic and eukaryotic cells utilizing promoters and other regulatory agents appropriate for the particular host cell. Exemplary host cells include, but are not limited to, E. coli, other bacterial hosts, yeast, and various higher eukaryotic cells such as the COS, CHO and HeLa cells lines and myeloma cell lines.

In mammalian host cells, a number of viral-based expression systems may be used, including retrovirus, lentivirus, adenovirus, adeno-associated virus, and the like. In cases where an adenovirus is used as an expression vector, the coding sequence of interest can be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the protein in infected hosts.

Specific initiation signals may also be required for efficient translation of the genes. These signals include the ATG initiation codon and adjacent sequences. In cases where a complete gene, including its own initiation codon and adjacent sequences, is inserted into the appropriate expression vector, no additional translational control signals may be needed. However, in cases where only a portion of the gene coding sequence is inserted, exogenous translational control signals must be provided. These exogenous translational control signals and initiation codons can be of a variety of origins, both natural and synthetic. The efficiency of expression may be enhanced by the inclusion of appropriate transcription enhancer elements, transcription terminators, etc.

In addition, a host cell strain may be chosen that modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, WI38, etc.

For long-term, production of recombinant proteins, stable expression is preferred. For example, cell lines that stably express endometrial target genes may be engineered. Rather than using expression vectors that contain viral origins of replication, host cells can be transformed with DNA controlled by appropriate expression control elements, and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines that express the target protein. Such engineered cell lines may be particularly useful in screening and evaluation of compounds that affect the endogenous activity of the protein. A number of selection systems may be used, including but not limited to the herpes simplex virus thymidine kinase, hypoxanthine-guanine phosphoribosyltransferase, and adenine phosphoribosyltransferase genes. Antimetabolite resistance can be used as the basis of selection for dhfr, which confers resistance to methotrexate; gpt, which confers resistance to mycophenolic acid; neo, which confers resistance to the aminoglycoside G-418; and hygro, which confers resistance to hygromycin.

The polypeptide may be labeled, either directly or indirectly. Any of a variety of suitable labeling systems may be used, including but not limited to, radioisotopes such as ¹²⁵I; enzyme labeling systems that generate a detectable colorimetric signal or light when exposed to substrate; and fluorescent labels. Indirect labeling involves the use of a protein, such as a labeled antibody, that specifically binds to the polypeptide of interest. Such antibodies include but are not limited to polyclonal, monoclonal, chimeric, single chain, Fab fragments and fragments produced by a Fab expression library.

Once expressed, the recombinant polypeptides can be purified according to standard procedures of the art, including ammonium sulfate precipitation, affinity columns, ion exchange and/or size exclusivity chromatography, gel electrophoresis and the like (see, generally, R. Scopes, Protein Purification, Springer—Overflag, N.Y. (1982), Deutsche, Methods in Enzymology Vol. 182: Guide to Protein Purification., Academic Press, Inc. N.Y. (1990)).

As an option to recombinant methods, polypeptides and oligopeptides can be chemically synthesized. Such methods typically include solid-state approaches, but can also utilize solution based chemistries and combinations or combinations of solid-state and solution approaches. Examples of solid-state methodologies for synthesizing proteins are described by Merrifield (1964) J. Am. Chem. Soc. 85:2149; and Houghton (1985) Proc. Natl. Acad. Sci., 82:5132. Fragments of an ischemia-associated protein can be synthesized and then joined together. Methods for conducting such reactions are described by Grant (1992) Synthetic Peptides: A User Guide, W. H. Freeman and Co., N.Y.; and in “Principles of Peptide Synthesis,” (Bodansky and Trost, ed.), Springer-Verlag, Inc. N.Y., (1993).

### Diagnostic and Prognostic Methods

The differential expression of the implantation window in endometriosis indicates that these can serve as markers for the diagnosis of endometriosis, for confirming fertility and infertility, and other physiological states of the endometrium. Diagnostic methods include detection of specific markers correlated with specific stages in the physiological processes involved in these states. Knowledge of the progression stage can be the basis for more accurate assessment of the most appropriate treatment and most appropriate administration of therapeutics.

In general, such diagnostic and prognostic methods involve detecting an altered level of expression of endometrial target transcripts or gene product in the cells or tissue of an individual or a sample therefrom. A variety of different assays can be utilized to detect an increase or decrease in endometrial target expression, including methods that detect gene transcript or protein levels. More specifically, the diagnostic and prognostic methods disclosed herein involve obtaining a sample from an individual and determining at least qualitatively, and preferably quantitatively, the level of a endometrial target expression in the sample. Usually this determined value or test value is compared against some type of reference or baseline value.

Nucleic acids or binding members such as antibodies that are specific for endometrial target polypeptides are used to screen patient samples for increased expression of the corresponding mRNA or protein, or for the presence of amplified DNA in the cell. Samples can be obtained from a variety of sources. For example, since the methods are designed primarily to diagnosis and assess risk factors for humans, samples are typically obtained from a human subject. However, the methods can also be utilized with samples obtained from various other mammals, such as primates, e.g. apes and chimpanzees, mice, cats, rats, and other animals. Such samples are referred to as a patient sample.

Samples can be obtained from the tissues or fluids of an individual, as well as from cell cultures or tissue homogenates. For example, samples can be obtained from whole blood, endometrial tissue scrapings, serum, semen, saliva, tears, urine, fecal material, sweat, buccal, skin, spinal fluid and amniotic fluid. Also included in the term are derivatives and fractions of such cells and fluids. Samples can also be derived from in vitro cell cultures, including the growth medium, recombinant cells and cell components. The number of cells in a sample will often be at least about 10², usually at least 10³, and may be about 10⁴ or more. The cells may be dissociated, in the case of solid tissues, or tissue sections may be analyzed. Alternatively a lysate of the cells may be prepared.

The various test values determined for a sample from an individual typically are compared against a baseline value or a control value to assess the extent of increased expression, if any. This baseline value can be any of a number of different values. In some embodiments, a baseline value is a value at a point in the menstrual cycle. In some embodiments, a control value is a level of a gene product at a given point in the menstrual cycle in a normal, healthy individual (e.g., an individual who does not have endometriosis). In some instances, the baseline value is a value established in a trial using a healthy cell or tissue sample that is run in parallel with the test sample. Alternatively, the baseline value can be a statistical value (e.g., a mean or average) established from a population of control cells or individuals. For example, the baseline value can be a value or range which is characteristic of a control individual or control population. For instance, the baseline value can be a statistical value or range that is reflective of expression levels for the general population, or more specifically, healthy individuals not affected with the condition being tested.

As discussed in the examples, a number of genes were identified that are differentially expressed during the window of implantation, as compared to other time points during the menstrual cycle, in normal women (e.g., women without endometriosis). Furthermore, certain genes were identified that are differentially expressed during the window of implantation in endometriosis (as compared to the level of expression during the window of implantation in normal women without endometriosis). Table 2 presents genes that are up-regulated (i.e., the level of mRNA is increased) during the window of implantation. Table 3 presents genes that a red own-regulated (i.e., the level of mRNA is decreased) during the window of implantation. Table 5 presents genes that are up-regulated during the window of implantation in women with endometriosis as compared to the level during the window of implantation in women without endometriosis. Table 6 presents genes that are down-regulated during the window of implantation in women with endometriosis as compared to the level during the window of implantation in women without endometriosis. In some embodiments, an mRNA level, or a level of a protein encoded by an mRNA, that is normally differentially expressed during the window of implantation is detected, and provides an indication as to whether the window of implantation has been reached, and of the likelihood of successful blastocyst implantation. For example, the level of expression any of the genes listed in Table 2 and/or Table 3 that is up-regulated or down-regulated during the window of implantation such that the level is increased or decreased by from about 2-fold to about 100-fold or more, e.g., from about 2-fold to about 5-fold, from about 5-fold to about 10-fold, from about 10-fold to about 20-fold, from about 20-fold to about 30-fold, from about 30-fold to about 40-fold, from about 40-fold to about 50-fold, from about 60-fold to about 70-fold, from about 70-fold to about 80-fold, from about 80-fold to about 90-fold, or from about 90-fold to about 100-fold or higher, can be detected. In other embodiments, an mRNA level, or a level of a protein encoded by an mRNA, that is differentially expressed in endometriosis during the window of implantation, is detected, and provides an indication as to whether the individual has endometriosis. For example, the level of expression of any of the genes listed in Table 5 and/or Table 6 that is up-regulated or down-regulated during the window of implantation in women with endometriosis such that the level is increased or decreased by from about 2-fold to about 100-fold or more, e.g., from about 2-fold to about 5-fold, from about 5-fold to about 10-fold, from about 10-fold to about 20-fold, from about 20-fold to about 30-fold, from about 30-fold to about 40-fold, from about 40-fold to about 50-fold, from about 60-fold to about 70-fold, from about 70-fold to about 80-fold, from about 80-fold to about 90-fold, or from about 90-fold to about 100-fold or higher, can be detected.

Detecting Endometriosis

In some embodiments, the invention provides a method for detecting endometriosis in an individual. The method generally involves determining the level of an mRNA or protein, which is differentially expressed in endometriosis, in a sample taken from an individual during the window of implantation (e.g., menstrual cycle days 20-24), and comparing the expression level to a control value, e.g., an expression level in an individual or a population of individuals without endometriosis. A substantially higher or lower than normal value indicates that the individual has endometriosis.

In some embodiments, the mRNA or protein level being detected is an mRNA or protein that is up-regulated significantly during the window of implantation in endometrium in women with endometriosis, and that is down-regulated during the normal window of implantation (e.g., in women without endometriosis). An increase in mRNA or protein level, when compared to a normal control, of 2-fold to 100-fold or more, e.g., from about 2-fold to about 5-fold, from about 5-fold to about 10-fold, from about 10-fold to about 20-fold, from about 20-fold to about 30-fold, from about 30-fold to about 40-fold, from about 40-fold to about 50-fold, from about 60-fold to about 70-fold, from about 70-fold to about 80-fold, from about 80-fold to about 90-fold, or from about 90-fold to about 100-fold or higher, indicates that the individual has endometriosis. Non-limiting examples of mRNAs having increased levels during the window of implantation in women with endometriosis, and that are normally down-regulated during the window of implantation include an mRNA listed in Table 5, semaphorin E mRNA, neuronal olfactomedin-related ER localized protein mRNA, and Sam68-like phosphotyrosine protein alpha mRNA. In those embodiments in which a protein level is detected, the protein encoded by an mRNA that is differentially expressed in endometriosis is detected. Non-limiting examples of suitable proteins include semaphorin E, neuronal olfactomedin-related ER localized protein, and Sam68-like phosphotyrosine protein alpha.

In other embodiments, the mRNA or protein level being detected is an mRNA or protein that is up-regulated during the window of implantation in women without endometriosis and that is significantly decreased during the window of implantation in women with endometriosis. A decrease in mRNA or protein level, when compared to a normal control, of 2-fold to 100-fold or more, e.g., from about 2-fold to about 5-fold, from about 5-fold to about 10-fold, from about 10-fold to about 20-fold, from about 20-fold to about 30-fold, from about 30-fold to about 40-fold, from about 40-fold to about 50-fold, from about 60-fold to about 70-fold, from about 70-fold to about 80-fold, from about 80-fold to about 90-fold, or from about 90-fold to about 100-fold or higher, indicates that the individual has endometriosis. Non-limiting examples of mRNA having decreased levels during the window of implantation in women with endometriosis and increased levels during the window of implantation in women without endometriosis include an mRNA listed in Table 6, IL-15 mRNA, proline-rich protein mRNA, B61 mRNA, Dickkopf-1 mRNA, glycodelin mRNA, GlcNAc6ST mRNA, G0S2 protein mRNA, and purine nucleoside phosphorylase mRNA. In those embodiments in which a protein level is detected, the protein encoded by an mRNA that is differentially expressed in endometriosis is detected. Non-limiting examples of suitable proteins include IL-15, proline-rich protein, B61, Dickkopf-1, glycodelin, GlcNAc6ST, G0S2 protein, and purine nucleoside phosphorylase.

In other embodiments, the mRNA or protein level being detected is an mRNA or protein that is down-regulated during the window of implantation in women without endometriosis, and that is further down-regulated during the window of implantation in women with endometriosis. A decrease in mRNA or protein level, when compared to a normal control, of 2-fold to 100-fold or more, e.g., from about 2-fold to about 5-fold, from about 5-fold to about 10-fold, from about 10-fold to about 20-fold, from about 20-fold to about 30-fold, from about 30-fold to about 40-fold, from about 40-fold to about 50-fold, from about 60-fold to about 70-fold, from about 70-fold to about 80-fold, from about 80-fold to about 90-fold, or from about 90-fold to about 100-fold or higher, indicates that the individual has endometriosis. Non-limiting examples of mRNA having decreased levels during the window of implantation in women without endometriosis, and having further decreased levels during the window of implantation in women with endometriosis include neuronal pentraxin II mRNA. In those embodiments in which a protein level is detected, the protein encoded by an mRNA that is differentially expressed in endometriosis is detected. Non-limiting examples of suitable proteins include neuronal pentraxin II.

In many embodiments, two or more mRNA that are differentially expressed in endometriosis are detected, and the levels compared to normal control values. For example, in some embodiments, from two to 50 (or more) different mRNAs are detected, e.g., from 2 to about 5, from about 5 to about 10, from about 10 to about 20, from about 20 to about 30, from about 30 to about 40, from about 40 to about 50, or more than 50, different mRNAs are detected, and the levels compared to normal controls.

In many embodiments, two or more proteins encoded by mRNAs that are differentially expressed in endometriosis are detected, and the levels compared to normal control values. For example, in some embodiments, from two to 50 (or more) different proteins are detected, e.g., from 2 to about 5, from about 5 to about 10, from about 10 to about 20, from about 20 to about 30, from about 30 to about 40, from about 40 to about 50, or more than 50, different proteins are detected, and the levels compared to normal controls.

In some embodiments, multiple samples are taken at various points in the menstrual cycle, and expression levels of mRNA or proteins that are differentially expressed in endometriosis are compared with control values, e.g., expression levels in individuals without endometriosis.

Detecting Uterine Receptivity

The present invention provides methods for detecting uterine receptivity to blastocyst implantation during the window of implantation. The present invention provides methods for determining the likelihood of success of implantation of a blastocyst into the uterine wall. The present invention provides methods of determining a probability of success with an assisted reproductive technology or a naturally achieved conception. The methods generally involve detecting a level of an mRNA or protein that is differentially expressed in endometriosis and/or that is differentially expressed during the normal menstrual cycle, and, based on the level compared to a normal control or standard value, determining the likelihood of successful blastocyst implantation.

Determination of the receptivity to implantation is of particular importance in techniques such as in vitro fertilization (IVF), embryo transfer, gamete intrafallopian transfer (GIFT), tubal embryo transfer (TET), intracytoplasmic sperm injection (ICSI) and intrauterine insemination (IUI). Determination of uterine receptivity is also important in determining optimal timing of achieving conception following sexual intercourse by couples attempting to conceive by sexual intercourse.

In some embodiments, the present invention provides a method of determining the probability of success of implantation following an assisted reproductive technology or naturally achieved conception. The methods generally involve determining the level, in a biological sample from an individual, of an mRNA or protein that is differentially expressed during the window of implantation. In some embodiments, the mRNA or protein level being detected is an mRNA or protein that is up-regulated (e.g., the level is increased) significantly during the window of implantation (e.g., as compared to other times during the menstrual cycle) in normal women. A significant increase in mRNA or protein level, when compared to the level of mRNA or protein produced during a period of time other than the window of implantation, or when compared to a control value, indicates an increased likelihood of successful implantation of a blastocyst. An increase in mRNA or protein level, when compared to a normal control, of 2-fold to 100-fold or more, e.g., from about 2-fold to about 5-fold, from about 5-fold to about 10-fold, from about 10-fold to about 20-fold, from about 20-fold to about 30-fold, from about 30-fold to about 40-fold, from about 40-fold to about 50-fold, from about 60-fold to about 70-fold, from about 70-fold to about 80-fold, from about 80-fold to about 90-fold, or from about 90-fold to about 100-fold or higher, indicates an increased likelihood of successful blastocyst implantation. In some of these embodiments, a control value is an average level of an mRNA or protein that is produced in normal women outside of the window of implantation. Non-limiting examples of mRNAs having increased levels during the window of implantation in control women (e.g., women without endometriosis) include an mRNA listed in Table 2, Dkk-1, IGFBP-1, GABA_{A} R π subunit, and glycodelin. Non-limiting examples of proteins suitable for detection include proteins encoded by one or more of Dkk-1, IGFBP-1, GABA_{A} R π subunit, and glycodelin.

In other embodiments, the mRNA or protein level being detected is an mRNA or protein that is down-regulated (e.g., the level is decreased) significantly during the window of implantation (e.g., as compared to other times during the menstrual cycle) in normal women. A significant decrease in mRNA or protein level, when compared to the level of mRNA or protein produced during a period of time other than the window of implantation, or when compared to a control value, indicates an increased likelihood of successful implantation of a blastocyst. A decrease in mRNA or protein level, when compared to a normal control, of 2-fold to 100-fold or more, e.g., from about 2-fold to about 5-fold, from about 5-fold to about 10-fold, from about 10-fold to about 20-fold, from about 20-fold to about 30-fold, from about 30-fold to about 40-fold, from about 40-fold to about 50-fold, from about 60-fold to about 70-fold, from about 70-fold to about 80-fold, from about 80-fold to about 90-fold, or from about 90-fold to about 100-fold or higher, indicates an increased likelihood of successful blastocyst implantation. In some of these embodiments, a control value is an average level of an mRNA or protein that is produced in normal women outside of the window of implantation. Non-limiting examples of mRNAs having decreased levels during the window of implantation in control women (e.g., women without endometriosis) include an mRNA listed in Table 3, PGRMC-1, matrilysin, and FrpHE. Non-limiting examples of proteins suitable for detection include proteins encoded by one or more of PGRMC-1, matrilysin, and FrpHE.

In some embodiments, the present invention provides a method of determining the probability of success of implantation following an assisted reproductive technology or naturally achieved conception. The methods generally involve determining, in a biological sample from an individual, the level of an mRNA or protein that is differentially expressed in endometriosis during the window of implantation. The level is compared to a standard. Deviation of the level of mRNA or protein from a normal control correlates with a decreased likelihood of success of blastocyst implantation. Thus, e.g., a deviation in an mRNA or protein level of 2-fold to 100-fold or more, e.g., from about 2-fold to about 5-fold, from about 5-fold to about 10-fold, from about 10-fold to about 20-fold, from about 20-fold to about 30-fold, from about 30-fold to about 40-fold, from about 40-fold to about 50-fold, from about 60-fold to about 70-fold, from about 70-fold to about 80-fold, from about 80-fold to about 90-fold, or from about 90-fold to about 100-fold or higher, when compared to a normal control, indicates a reduced likelihood of successful blastocyst implantation. A level of an mRNA or protein that is differentially expressed in endometriosis that deviates from a normal control value by less than about 20-fold to less than about 10-fold, by less than about 10-fold to less than about 5-fold, or by less than about 5-fold to less than about 2-fold, indicates a greater likelihood of successful blastocyst implantation.

In some embodiments, the mRNA or protein level being detected is an mRNA or protein that is up-regulated significantly during the window of implantation in endometrium in women with endometriosis, and that is down-regulated during the normal window of implantation (e.g., in women without endometriosis). An increase in mRNA or protein level, when compared to a normal control, of 2-fold to 100-fold or more, e.g., from about 2-fold to about 5-fold, from about 5-fold to about 10-fold, from about 10-fold to about 20-fold, from about 20-fold to about 30-fold, from about 30-fold to about 40-fold, from about 40-fold to about 50-fold, from about 60-fold to about 70-fold, from about 70-fold to about 80-fold, from about 80-fold to about 90-fold, or from about 90-fold to about 100-fold or higher, indicates a reduced likelihood of successful blastocyst implantation. A level of an mRNA or protein that is differentially expressed in endometriosis that deviates from a normal control value by less than about 20-fold to less than about 10-fold, by less than about 10-fold to less than about 5-fold, or by less than about 5-fold to less than about 2-fold, indicates a greater likelihood of successful blastocyst implantation. Non-limiting examples of mRNAs having increased levels during the window of implantation in women with endometriosis, and that are normally down-regulated during the window of implantation include an mRNA listed in Table 5, semaphorin E mRNA, neuronal olfactomedin-related ER localized protein mRNA, and Sam68-like phosphotyrosine protein alpha mRNA. In those embodiments in which a protein level is detected, the protein encoded by an mRNA that is differentially expressed in endometriosis is detected. Non-limiting examples of suitable proteins include a protein encoded by an mRNA listed in Table 5, semaphorin E, neuronal olfactomedin-related ER localized protein, and Sam68-like phosphotyrosine protein alpha.

In other embodiments, the mRNA or protein level being detected is an mRNA or protein that is up-regulated during the window of implantation in women without endometriosis and that is significantly decreased during the window of implantation in women with endometriosis. A decrease in mRNA or protein level, when compared to a normal control, of 2-fold to 100-fold or more, e.g., from about 2-fold to about 5-fold, from about 5-fold to about 10-fold, from about 10-fold to about 20-fold, from about 20-fold to about 30-fold, from about 30-fold to about 40-fold, from about 40-fold to about 50-fold, from about 60-fold to about 70-fold, from about 70-fold to about 80-fold, from about 80-fold to about 90-fold, or from about 90-fold to about 100-fold or higher, indicates a reduced likelihood of successful blastocyst implantation. A level of an mRNA or protein that is differentially expressed in endometriosis that deviates from a normal control value by less than about 20-fold to less than about 10-fold, by less than about 10-fold to less than about 5-fold, or by less than about 5-fold to less than about 2-fold, indicates a greater likelihood of successful blastocyst implantation. Non-limiting examples of mRNA having decreased levels during the window of implantation in women with endometriosis and increased levels during the window of implantation in women without endometriosis include an mRNA listed in Table 6, IL-15 mRNA, proline-rich protein mRNA, B61 mRNA, Dickkopf-1 mRNA, glycodelin mRNA, GlcNAc6ST mRNA, G0S2 protein mRNA, and purine nucleoside phosphorylase mRNA. In those embodiments in which a protein level is detected, the protein encoded by an mRNA that is differentially expressed in endometriosis is detected. Non-limiting examples of suitable proteins include a protein encoded by an mRNA listed in Table 6, IL-15, proline-rich protein, B61, Dickkopf-1, glycodelin, GlcNAc6ST, G0S2 protein, and purine nucleoside phosphorylase.

In other embodiments, the mRNA or protein level being detected is an mRNA or protein that is down-regulated during the window of implantation in women without endometriosis, and that is further down-regulated during the window of implantation in women with endometriosis. A decrease in mRNA or protein level, when compared to a normal control, of 2-fold to 100-fold or more, e.g., from about 2-fold to about 5-fold, from about 5-fold to about 10-fold, from about 10-fold to about 20-fold, from about 20-fold to about 30-fold, from about 30-fold to about 40-fold, from about 40-fold to about 50-fold, from about 60-fold to about 70-fold, from about 70-fold to about 80-fold, from about 80-fold to about 90-fold, or from about 90-fold to about 100-fold or higher, indicates a reduced likelihood of successful blastocyst implantation. A level of an mRNA or protein that is differentially expressed in endometriosis that deviates from a normal control value by less than about 20-fold to less than about 10-fold, by less than about 10-fold to less than about 5-fold, or by less than about 5-fold to less than about 2-fold, indicates a greater likelihood of successful blastocyst implantation. Non-limiting examples of mRNA having decreased levels during the window of implantation in women without endometriosis, and having further decreased levels during the window of implantation in women with endometriosis include neuronal pentraxin II mRNA. In those embodiments in which a protein level is detected, the protein encoded by an mRNA that is differentially expressed in endometriosis is detected. Non-limiting examples of suitable proteins include neuronal pentraxin II.

In many embodiments, two or more mRNA that are differentially expressed in endometriosis are detected, and the levels compared to normal control values. For example, in some embodiments, from two to 50 (or more) different mRNAs are detected, e.g., from 2 to about 5, from about 5 to about 10, from about 10 to about 20, from about 20 to about 30, from about 30 to about 40, from about 40 to about 50, or more than 50, different mRNAs are detected, and the levels compared to normal controls.

In many embodiments, two or more proteins encoded by mRNAs that are differentially expressed in endometriosis are detected, and the levels compared to normal control values. For example, in some embodiments, from two to 50 (or more) different proteins are detected, e.g., from 2 to about 5, from about 5 to about 10, from about 10 to about 20, from about 20 to about 30, from about 30 to about 40, from about 40 to about 50, or more than 50, different proteins are detected, and the levels compared to normal controls.

In some embodiments, the invention provides methods of determining the window of implantation, e.g., for determining the optimal timing for blastocyst implantation. Such methods are useful for determining the optimal timing for an assisted reproduction technology. Such methods are also useful for home use, to determine the optimal timing for achieving conception naturally. The methods generally involve detecting a level of an mRNA or protein that is differentially expressed during a normal menstrual cycle. The level is compared to a normal control value. A level of an mRNA or protein, which is differentially expressed during the normal menstrual cycle, that is at or near the normal level produced during the window of implantation indicates that the likelihood of achieving conception following sexual intercourse is increased relative to other times during the cycle.

In some embodiments, the mRNA or protein level being detected is an mRNA or protein that is up-regulated significantly (e.g., the level is increased) during the window of implantation in women without endometriosis (e.g., normal controls). A level of an mRNA or protein, which is differentially expressed during the window of implantation, that deviates from a normal control value by less than about 20-fold to less than about 10-fold, by less than about 10-fold to less than about 5-fold, or by less than about 5-fold to less than about 2-fold, indicates a greater likelihood of successful blastocyst implantation. Non-limiting examples of mRNA that are up-regulated during the window of implantation in normal controls include an mRNA listed in Table 2, Dkk-1, IGFBP-1, GABA_{A} R π subunit, and glycodelin. In those embodiments in which a protein level is detected, the protein encoded by an mRNA that is differentially expressed during the window of implantation in normal controls is detected.

In some embodiments, the mRNA or protein level being detected is an mRNA or protein that is down-regulated significantly (e.g., the level is decreased) during the window of implantation in women without endometriosis (e.g., normal controls). A level of an mRNA or protein, which is differentially expressed during the window of implantation, that deviates from a normal control value by less than about 20-fold to less than about 10-fold, by less than about 10-fold to less than about 5-fold, or by less than about 5-fold to less than about 2-fold, indicates a greater likelihood of successful blastocyst implantation. Non-limiting examples of mRNA that are down-regulated during the window of implantation in normal controls include an mRNA listed in Table 3, PGRMC-1, matrilysin, and FrpHE. In those embodiments in which a protein level is detected, the protein encoded by an mRNA that is differentially expressed during the window of implantation in normal controls is detected.

In some embodiments, the mRNA or protein level being detected is an mRNA or protein that is up-regulated significantly during the window of implantation in endometrium in women with endometriosis, and that is down-regulated during the normal window of implantation (e.g., in women without endometriosis). A level of an mRNA or protein, which is differentially expressed during the window of implantation, that deviates from a normal control value by less than about 20-fold to less than about 10-fold, by less than about 10-fold to less than about 5-fold, or by less than about 5-fold to less than about 2-fold, indicates a greater likelihood of successful blastocyst implantation. Non-limiting examples of mRNAs having increased levels during the window of implantation in women with endometriosis, and that are normally down-regulated during the window of implantation include an mRNA listed in Table 5, semaphorin E mRNA, neuronal olfactomedin-related ER localized protein mRNA, and Sam68-like phosphotyrosine protein alpha mRNA. In those embodiments in which a protein level is detected, the protein encoded by an mRNA that is differentially expressed in endometriosis is detected. Non-limiting examples of suitable proteins include a protein encoded by an mRNA listed in Table 5, semaphorin E, neuronal olfactomedin-related ER localized protein, and Sam68-like phosphotyrosine protein alpha.

In other embodiments, the mRNA or protein level being detected is an mRNA or protein that is up-regulated during the window of implantation in women without endometriosis and that is significantly decreased during the window of implantation in women with endometriosis. A level of an mRNA or protein, which is differentially expressed during the window of implantation, that deviates from a normal control value by less than about 20-fold to less than about 10-fold, by less than about 10-fold to less than about 5-fold, or by less than about 5-fold to less than about 2-fold, indicates a greater likelihood of successful blastocyst implantation. Non-limiting examples of mRNA having decreased levels during the window of implantation in women with endometriosis and increased levels during the window of implantation in women without endometriosis include an mRNA listed in Table 6, IL-15 mRNA, proline-rich protein mRNA, B61 mRNA, Dickkopf-1 mRNA, glycodelin mRNA, GlcNAc6ST mRNA, G0S2 protein mRNA, and purine nucleoside phosphorylase mRNA. In those embodiments in which a protein level is detected, the protein encoded by an mRNA that is differentially expressed in endometriosis is detected. Non-limiting examples of suitable proteins include a protein encoded by an mRNA listed in Table 6, IL-15, proline-rich protein, B61, Dickkopf-1, glycodelin, GlcNAc6ST, G0S2 protein, and purine nucleoside phosphorylase.

In other embodiments, the mRNA or protein level being detected is an mRNA or protein that is down-regulated during the window of implantation in women without endometriosis, and that is further down-regulated during the window of implantation in women with endometriosis. A level of an mRNA or protein, which is differentially expressed during the window of implantation, that deviates from a normal control value by less than about 20-fold to less than about 10-fold, by less than about 10-fold to less than about 5-fold, or by less than about 5-fold to less than about 2-fold, indicates a greater likelihood-of successful blastocyst implantation. Non-limiting examples of mRNA having decreased levels during the window of implantation in women without endometriosis, and having further decreased levels during the window of implantation in women with endometriosis include neuronal pentraxin II mRNA. In those embodiments in which a protein level is detected, the protein encoded by an mRNA that is differentially expressed in endometriosis is detected. Non-limiting examples of suitable proteins include neuronal pentraxin II.

In many embodiments, two or more mRNA that are differentially expressed in endometriosis are detected, and the levels compared to normal control values. For example, in some embodiments, from two to 50 (or more) different mRNAs are detected, e.g., from 2 to about 5, from about 5 to about 10, from about 10 to about 20, from about 20 to about 30, from about 30 to about 40, from about 40 to about 50, or more than 50, different mRNAs are detected, and the levels compared to normal controls.

In many embodiments, two or more proteins encoded by mRNAs that are differentially expressed in endometriosis are detected, and the levels compared to normal control values. For example, in some embodiments, from two to 50 (or more) different proteins are detected, e.g., from 2 to about 5, from about 5 to about 10, from about 10 to about 20, from about 20 to about 30, from about 30 to about 40, from about 40 to about 50, or more than 50, different proteins are detected, and the levels compared to normal controls.

In many embodiments, multiple samples taken, e.g., on 2, 3, 4, 5, 6, 7, or more success days are tested, and the optimal timing for a naturally-achieved conception is determined by comparing the level of mRNA or protein between the levels produced on two or more successive days.

Nucleic Acid Screening Methods

Some of the diagnostic and prognostic methods that involve the detection of an endometrial target transcript begin with the lysis of cells and subsequent purification of nucleic acids from other cellular material, particularly mRNA transcripts. A nucleic acid derived from an mRNA transcript refers to a nucleic acid for whose synthesis the mRNA transcript, or a subsequence thereof, has ultimately served as a template. Thus, a cDNA reverse transcribed from an mRNA, an RNA transcribed from that cDNA, a DNA amplified from the cDNA, an RNA transcribed from the amplified DNA, are all derived from the mRNA transcript and detection of such derived products is indicative of the presence and/or abundance of the original transcript in a sample. Thus, suitable samples include, but are not limited to, mRNA transcripts, cDNA reverse transcribed from the mRNA, cRNA transcribed from the cDNA, DNA amplified from nucleic acids, and RNA transcribed from amplified DNA.

A number of methods are available for analyzing nucleic acids for the presence of a specific sequence, e.g. upregulated or downregulated expression. The nucleic acid may be amplified by conventional techniques, such as the polymerase chain reaction (PCR), to provide sufficient amounts for analysis. The use of the polymerase chain reaction is described in Saiki et al. (1985) Science 239:487, and a review of techniques may be found in Sambrook, et al. Molecular Cloning: A Laboratory Manual, CSH Press 1989, pp.14.2-14.33.

A detectable label may be included in an amplification reaction. Suitable labels include fluorochromes, e.g. fluorescein isothiocyanate (FITC), rhodamine, Texas Red, phycoerythrin, allophycocyanin,6-carboxyfluorescein(6-FAM),2,7-dimethoxy-4,5-dichloro-6-carboxyfluorescein (JOE), 6-carboxy-X-rhodamine (ROX), 6-carboxy-2,4,7,4,7-hexachlorofluorescein (HEX), 5-carboxyfluorescein (5-FAM) or N,N,N,N-tetramethyl-6-carboxyrhodamine (TAMRA), radioactive labels, e.g. ³²P, ³⁵S, ³H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

The sample nucleic acid, e.g. amplified, labeled, cloned fragment, etc. is analyzed by one of a number of methods known in the art. Probes may be hybridized to northern or dot blots, or liquid hybridization reactions performed. The nucleic acid may be sequenced by dideoxy or other methods, and the sequence of bases compared to a wild-type sequence.

Single strand conformational polymorphism (SSCP) analysis, denaturing gradient gel electrophoresis (DGGE), and heteroduplex analysis in gel matrices are used to detect conformational changes created by DNA sequence variation as alterations in electrophoretic mobility. Fractionation is performed by gel or capillary electrophoresis, particularly acrylamide or agarose gels.

In situ hybridization methods are hybridization methods in which the cells are not lysed prior to hybridization. Because the method is performed in situ, it has the advantage that it is not necessary to prepare RNA from the cells. The method usually involves initially fixing test cells to a support (e.g., the walls of a microtiter well) and then permeabilizing the cells with an appropriate permeabilizing solution. A solution containing labeled probes is then contacted with the cells and the probes allowed to hybridize. Excess probe is digested, washed away and the amount of hybridized probe measured. This approach is described in greater detail by Harris, D. W. (1996) Anal. Biochem. 243:249-256; Singer, et al. (1986) Biotechniques 4:230-250; Haase et al. (1984) Methods in Virology, vol. VII, pp. 189-226; and Nucleic Acid Hybridization: A Practical Approach (Hames, et al., eds., 1987).

A variety of so-called “real time amplification” methods or “real time quantitative PCR” methods can also be utilized to determine the quantity mRNA present in a sample. Such methods involve measuring the amount of amplification product formed during an amplification process. Fluorogenic nuclease assays are one specific example of a real time quantitation method that can be used to detect and quantitate transcripts. In general such assays continuously measure PCR product accumulation using a dual-labeled fluorogenic oligonucleotide probe—an approach frequently referred to in the literature simply as the “TaqMan” method.

The probe used in such assays is typically a short (ca. 20-25 bases) polynucleotide that is labeled with two different fluorescent dyes. The 5′ terminus of the probe is typically attached to a reporter dye and the 3′ terminus is attached to a quenching dye, although the dyes can be attached at other locations on the probe as well. For measuring transcript levels, the probe is designed to have at least substantial sequence complementarity with the target sequence. Upstream and downstream PCR primers that bind to regions that flank the target gene are also added to the reaction mixture. Probes may also be made by in vitro transcription methods.

When the probe is intact, energy transfer between the two fluorophors occurs and the quencher quenches emission from the reporter. During the extension phase of PCR, the probe is cleaved by the 5′ nuclease activity of a nucleic acid polymerase such as Taq polymerase, thereby releasing the reporter dye from the polynucleotide-quencher complex and resulting in an increase of reporter emission intensity that can be measured by an appropriate detection system.

One detector which is specifically adapted for measuring fluorescence emissions such as those created during a fluorogenic assay is the ABI 7700 manufactured by Applied Biosystems, Inc. in Foster City, Calif. Computer software provided with the instrument is capable of recording the fluorescence intensity of reporter and quencher over the course of the amplification. These recorded values can then be used to calculate the increase in normalized reporter emission intensity on a continuous basis and ultimately quantify the amount of the mRNA being amplified.

Polypeptide Screening Methods

Screening for expression of the subject sequences may be based on the functional or antigenic characteristics of the protein. Protein truncation assays are useful in detecting deletions that may affect the biological activity of the protein. Various immunoassays designed to detect polymorphisms in proteins encoded by the target genes may be used in screening. Where many diverse genetic mutations lead to a particular disease phenotype, functional protein assays have proven to be effective screening tools. The activity of the encoded protein in protein assays, etc., may be determined by comparison with the wild-type protein.

Detection may utilize staining of cells or histological sections, performed in accordance with conventional methods, using antibodies or other specific binding members. The antibodies or other specific binding members of interest are added to a cell sample, and incubated for a period of time sufficient to allow binding to the epitope, usually at least about 10 minutes. The antibody may be labeled with radioisotopes, enzymes, fluorescers, chemiluminescers, or other labels for direct detection. Alternatively, a second stage antibody or reagent is used to amplify the signal. Such reagents are well known in the art. For example, the primary antibody may be conjugated to biotin, with horseradish peroxidase-conjugated avidin added as a second stage reagent. Final detection uses a substrate that undergoes a color change in the presence of the peroxidase. The absence or presence of antibody binding may be determined by various methods, including flow cytometry of dissociated cells, microscopy, radiography, scintillation counting, etc.

An alternative method for diagnosis depends on the in vitro detection of binding between antibodies and polypeptide in a lysate. Measuring the concentration of the target protein in a sample or fraction thereof may be accomplished by a variety of specific assays. A conventional sandwich type assay may be used. For example, a sandwich assay may first attach specific antibodies to an insoluble surface or support. The particular manner of binding is not crucial so long as it is compatible with the reagents and overall methods of the invention. They may be bound to the plates covalently or non-covalently, preferably non-covalently.

The insoluble supports may be any compositions to which polypeptides can be bound, which is readily separated from soluble material, and which is otherwise compatible with the overall method. The surface of such supports may be solid or porous and of any convenient shape. Examples of suitable insoluble supports to which the receptor is bound include beads, e.g. magnetic beads, membranes and microtiter plates. These are typically made of glass, plastic (e.g. polystyrene), polysaccharides, nylon or nitrocellulose. Microtiter plates are especially convenient because a large number of assays can be carried out simultaneously, using small amounts of reagents and samples.

Patient sample lysates are then added to separately assayable supports (for example, separate wells of a microtiter plate) containing antibodies. Preferably, a series of standards, containing known concentrations of the test protein is assayed in parallel with the samples or aliquots thereof to serve as controls. Preferably, each sample and standard will be added to multiple wells so that mean values can be obtained for each. The incubation time should be sufficient for binding, generally, from about 0.1 to 3 hr is sufficient. After incubation, the insoluble support is generally washed of non-bound components. Generally, a dilute non-ionic detergent medium at an appropriate pH, generally 7-8, is used as a wash medium. From one to six washes may be employed, with sufficient volume to thoroughly wash non-specifically bound proteins present in the sample.

After washing, a solution containing a second antibody is applied. The antibody will bind to one of the proteins of interest with sufficient specificity such that it can be distinguished from other components present. The second antibodies may be labeled to facilitate direct, or indirect quantification of binding. Examples of labels that permit direct measurement of second receptor binding include radiolabels, such as ³H or ¹²⁵I, fluorescers, dyes, beads, chemiluminescers, colloidal particles, and the like. Examples of labels that permit indirect measurement of binding include enzymes where the substrate may provide for a colored or fluorescent product. In a preferred embodiment, the antibodies are labeled with a covalently bound enzyme capable of providing a detectable product signal after addition of suitable substrate. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art. The incubation time should be sufficient for the labeled ligand to bind available molecules. Generally, from about 0.1 to 3 hr is sufficient, usually 1 hr sufficing.

After the second binding step, the insoluble support is again washed free of non-specifically bound material, leaving the specific complex formed between the target protein and the specific binding member. The signal produced by the bound conjugate is detected by conventional means. Where an enzyme conjugate is used, an appropriate enzyme substrate is provided so a detectable product is formed.

Other immunoassays are known in the art and may find use as diagnostics. Ouchterlony plates provide a simple determination of antibody binding. Western blots may be performed on protein gels or protein spots on filters, using a detection system specific for the ischemia associated polypeptide, or ischemia pathway polypeptide as desired, conveniently using a labeling method as described for the sandwich assay.

In some cases, a competitive assay will be used. In addition to the patient sample, a competitor to the targeted protein is added to the reaction mix. The competitor and the ischemia associated polypeptide, or ischemia pathway polypeptide compete for binding to the specific binding partner. Usually, the competitor molecule will be labeled and detected as previously described, where the amount of competitor binding will be proportional to the amount of target protein present. The concentration of competitor molecule will be from about 10 times the maximum anticipated protein concentration to about equal concentration in order to make the most sensitive and linear range of detection.

In some embodiments, the methods are adapted for use in vivo, e.g., to locate or identify sites where cells of interest are present. In these embodiments, a detectably-labeled moiety, e.g., an antibody, is administered to an individual (e.g., by injection), and labeled cells are located using standard imaging techniques, including, but not limited to, magnetic resonance imaging, computed tomography scanning, and the like.

The detection methods can be provided as part of a kit. Thus, the invention further provides kits for detecting the presence of mRNA, and/or a polypeptide encoded thereby, in a biological sample. Procedures using these kits can be performed by clinical laboratories, experimental laboratories, medical practitioners, or private individuals. The kits of the invention for detecting a polypeptide comprise a moiety that specifically binds the polypeptide, which may be a specific antibody. The kits of the invention for detecting a nucleic acid comprise a moiety that specifically hybridizes to such a nucleic acid. The kit may optionally provide additional components that are useful in the procedure, including, but not limited to, buffers, developing reagents, labels, reacting surfaces, means for detection, control samples, standards, instructions, and interpretive information.

Time Course Analyses

Certain prognostic and diagnostic methods involve monitoring expression levels for a patient susceptible to endometrial disorders, to track whether there is an alteration in expression of an endometrial target genes over time. As with other measures, the expression level for the patient being tested for endometriosis and/or fertility status is compared against a baseline value. The baseline in such analyses can be a prior value determined for the same individual or a statistical value (e.g., mean or average) determined for a control group (e.g., a population of individuals with no history of endometriosis and/or no history of infertility). An individual showing a statistically significant increase in expression levels over time can prompt the individual's physician to take prophylactic measures.

### Therapeutic/Prophylactic Treatment Methods

Agents that modulate activity of endometrial target genes provide a point of therapeutic or prophylactic intervention. Numerous agents are useful in modulating this activity, including agents that directly modulate expression, e.g. expression vectors, antisense specific for the targeted protein; and agents that act on the protein, e.g. specific antibodies and analogs thereof, small organic molecules that block catalytic activity, etc.

The genes, gene fragments, or the encoded protein or protein fragments are useful in therapy to treat disorders associated with defects in sequence or expression. From a therapeutic point of view, modulating activity has a therapeutic effect on a number of disorders. Antisense sequences may be administered to inhibit expression. Pseudo-substrate inhibitors, for example, a peptide that mimics a substrate for the protein may be used to inhibit activity. Other inhibitors are identified by screening for biological activity in a functional assay, e.g. in vitro or in vivo protein activity. Alternatively, expression can be upregulated by introduction of an expression vector, enhancing expression, providing molecules that mimic the activity of the targeted polypeptide, etc.

Expression vectors may be used to introduce the target gene into a cell. Such vectors generally have convenient restriction sites located near the promoter sequence to provide for the insertion of nucleic acid sequences. Transcription cassettes may be prepared comprising a transcription initiation region, the target gene or fragment thereof, and a transcriptional termination region. The transcription cassettes may be introduced into a variety of vectors, e.g. plasmid; retrovirus, e.g. lentivirus; adenovirus; and the like, where the vectors are able to transiently or stably be maintained in the cells, usually for a period of at least about one day, more usually for a period of at least about several days to several weeks.

The gene or protein may be introduced into tissues or host cells by any number of routes, including viral infection, microinjection, or fusion of vesicles. Jet injection may also be used for intramuscular administration, as described by Furth et al. (1992) Anal Biochem 205:365-368. The DNA may be coated onto gold microparticles, and delivered intradermally by a particle bombardment device, or “gene gun” as described in the literature (see, for example, Tang et al. (1992) Nature 356:152-154), where gold micro projectiles are coated with the protein or DNA, then bombarded into cells.

When liposomes are utilized, substrates that bind to a cell-surface membrane protein associated with endocytosis can be attached to the liposome to target the liposome to nerve cells and to facilitate uptake. Examples of proteins that can be attached include capsid proteins or fragments thereof that bind to nerve cells, antibodies that specifically bind to cell-surface proteins on nerve cells that undergo internalization in cycling and proteins that target intracellular localizations within cells. Gene marking and gene therapy protocols are reviewed by Anderson et al. (1992) Science 256:808-813.

Antisense molecules can be used to down-regulate expression in cells. The antisense reagent may be antisense oligonucleotides (ODN), particularly synthetic ODN having chemical modifications from native nucleic acids, or nucleic acid constructs that express such antisense molecules as RNA. The antisense sequence is complementary to the mRNA of the targeted gene, and inhibits expression of the targeted gene products. Antisense molecules inhibit gene expression through various mechanisms, e.g. by reducing the amount of mRNA available for translation, through activation of RNAse H, or steric hindrance. One or a combination of antisense molecules may be administered, where a combination may comprise multiple different sequences.

Antisense molecules may be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like. It has been found that short oligonucleotides, of from 7 to 8 bases in length, can be strong and selective inhibitors of gene expression (see Wagner et al. (1996) Nature Biotechnology 14:840-844).

A specific region or regions of the endogenous sense strand mRNA sequence is chosen to be complemented by the antisense sequence. Selection of a specific sequence for the oligonucleotide may use an empirical method, where several candidate sequences are assayed for inhibition of expression of the target gene in vitro or in an animal model. A combination of sequences may also be used, where several regions of the mRNA sequence are selected for antisense complementation.

Antisense oligonucleotides may be chemically synthesized by methods known in the art (see Wagner et al. (1993) supra. and Milligan et al., supra.) Preferred oligonucleotides are chemically modified from the native phosphodiester structure, in order to increase their intracellular stability and binding affinity. A number of such modifications have been described in the literature, which alter the chemistry of the backbone, sugars or heterocyclic bases.

Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH2-5′-O-phosphonate and 3′-NH-5′-O-phosphoroamidate. Peptide nucleic acids replace the entire ribose phosphodiester backbone with a peptide linkage. Sugar modifications are also used to enhance stability and affinity. The α-anomer of deoxyribose may be used, where the base is inverted with respect to the natural β-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

### Compound Screening

Compound screening may be performed using an in vitro model, an in vitro eukaryotic cell (e.g., an endometrial cell), a genetically altered cell or animal, or purified protein. One can identify ligands or substrates that bind to, modulate or mimic the action of the encoded polypeptide.

The polypeptides include those encoded by the provided endometrial target genes, as well as nucleic acids that, by virtue of the degeneracy of the genetic code, are not identical in sequence to the disclosed nucleic acids, and variants thereof. Variant polypeptides can include amino acid (aa) substitutions, additions or deletions. The amino acid substitutions can be conservative amino acid substitutions or substitutions to eliminate non-essential amino acids, such as to alter a glycosylation site, a phosphorylation site or an acetylation site, or to minimize misfolding by substitution or deletion of one or more cysteine residues that are not necessary for function. Variants can be designed so as to retain or have enhanced biological activity of a particular region of the protein (e.g., a functional domain and/or, where the polypeptide is a member of a protein family, a region associated with a consensus sequence). Variants also include fragments of the polypeptides disclosed herein, particularly biologically active fragments and/or fragments corresponding to functional domains. Fragments of interest will typically be at least about 10 aa to at least about 15 aa in length, usually at least about 50 aa in length, and can be as long as 300 aa in length or longer, but will usually not exceed about 500 aa in length, where the fragment will have a contiguous stretch of amino acids that is identical to a polypeptide encoded by an endometrial target gene, or a homolog thereof.

Transgenic animals or cells derived therefrom are also used in compound screening. Transgenic animals may be made through homologous recombination, where the normal locus is altered. Alternatively, a nucleic acid construct is randomly integrated into the genome. Vectors for stable integration include plasmids, retroviruses and other animal viruses, YACs, and the like. A series of small deletions and/or substitutions may be made in the coding sequence to determine the role of different exons in protein activity, signal transduction, etc. Specific constructs of interest include antisense sequences that block expression of the targeted gene and expression of dominant negative mutations. A detectable marker, such as lac Z may be introduced into the locus of interest, where up-regulation of expression will result in an easily detected change in phenotype. One may also provide for expression of the target gene or variants thereof in cells or tissues where it is not normally expressed or at abnormal times of development. By providing expression of the target protein in cells in which it is not normally produced, one can induce changes in cell behavior.

In some embodiments, a subject screening method identifies agents that modulate a level of an endometrial mRNA and/or polypeptide, wherein the endometrial mRNA is one that is differentially expressed during the window of implantation. In some embodiments, the methods involve contacting an endometrial cell in vitro with a test agent (a “candidate agent”); and determining the effect, if any, of the test agent on the level of the differentially expressed mRNA. In some embodiments, the methods involve contacting a eukaryotic cell with a test agent, where the eukaryotic cell is genetically modified with a construct that comprises a nucleotide sequence that encodes a differentially expressed mRNA; and determining the effect, if any, of the test agent on the level of the differentially expressed mRNA. The level of an mRNA is detected using any known method, including a hybridization-based method using a detectably-labeled nucleic acid that hybridizes to a differentially expressed mRNA; and the like. An agent that modulates a level of an mRNA that is differentially expressed during the window of implantation is a candidate agent for the treatment of endometrial disorders, including endometriosis, and in some embodiments is a candidate contraceptive.

Compound screening identifies agents that modulate a level or a function of an endometrial target mRNA and/or polypeptide. Of particular interest are screening assays for agents that have a low toxicity for human cells. A wide variety of assays may be used for this purpose, including labeled in vitro protein-protein binding assays, electrophoretic mobility shift assays, immunoassays for protein binding, and the like. Knowledge of the 3-dimensional structure of the encoded protein, derived from crystallization of purified recombinant protein, could lead to the rational design of small drugs that specifically inhibit activity. These drugs may be directed at specific domains.

The term “agent” as used herein describes any molecule, e.g. protein or pharmaceutical, with the capability of altering or mimicking the physiological function. Generally a plurality of assay mixtures are run in parallel with different agent concentrations to obtain a differential response to the various concentrations. Typically one of these concentrations serves as a negative control, i.e. at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Test agents can be obtained from libraries, such as natural product libraries or combinatorial libraries, for example. A number of different types of combinatorial libraries and methods for preparing such libraries have been described, including for example, PCT publications WO 93/06121, WO 95/12608, WO 95/35503, WO 94/08051 and WO 95/30642, each of which is incorporated herein by reference.

Where the screening assay is a binding assay, one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin, etc. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used. The mixture of components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hours will be sufficient.

Preliminary screens can be conducted by screening for compounds capable of binding to an endometrial target polypeptide, as at least some of the compounds so identified are likely inhibitors. The binding assays usually involve contacting a protein with one or more test compounds and allowing sufficient time for the protein and test compounds to form a binding complex. Any binding complexes formed can be detected using any of a number of established analytical techniques. Protein binding assays include, but are not limited to, methods that measure co-precipitation, co-migration on non-denaturing SDS-polyacrylamide gels, and co-migration on Western blots.

Certain screening methods involve screening for a compound that modulates the expression of a gene. Such methods generally involve conducting cell-based assays in which test compounds are contacted with one or more cells expressing an endometrial target polypeptide and then detecting an increase in gene expression (either transcript or translation product).

Compounds that are initially identified by any of the foregoing screening methods can be further tested to validate the apparent activity. The basic format of such methods involves administering a lead compound identified during an initial screen to an animal that serves as a model for humans and then determining if the target gene is in fact upregulated. The animal models utilized in validation studies generally are mammals. Specific examples of suitable animals include, but are not limited to, primates, mice, and rats.

### Pharmaceutical Compositions

Compounds identified by the screening methods described above and analogs thereof can serve as the active ingredient in pharmaceutical compositions formulated for the treatment of various disorders. The compositions can also include various other agents to enhance delivery and efficacy. The compositions can also include various agents to enhance delivery and stability of the active ingredients.

Thus, for example, the compositions can also include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mack Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture and/or animal studies can be used in formulating a range of dosages for humans. The dosage of the active ingredient typically lines within a range of circulating concentrations that include the ED₅₀ with little or no toxicity. The dosage can vary within this range depending upon the dosage form employed and the route of administration utilized.

The pharmaceutical compositions described herein can be administered in a variety of different ways. Examples include administering a composition containing a pharmaceutically acceptable carrier via oral, intranasal, rectal, topical, intraperitoneal, intravenous, intramuscular, subcutaneous, subdermal, transdermal and intrathecal methods.

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

The active ingredient, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen.

Suitable formulations for rectal administration include, for example, suppositories, which consist of the packaged active ingredient with a suppository base. Suitable suppository bases include natural or synthetic triglycerides or paraffin hydrocarbons. In addition, it is also possible to use gelatin rectal capsules which consist of a combination of the packaged active ingredient with a base, including, for example, liquid triglycerides, polyethylene glycols, and paraffin hydrocarbons.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

Kits

Also provided are reagents and kits thereof for practicing one or more of the above-described methods. The subject reagents and kits thereof may vary greatly. Reagents of interest include reagents specifically designed for use in production of the above described expression profiles of phenotype determinative genes.

A subject kit includes one or more binding agents that specifically bind an mRNA or protein that is differentially expressed in endometriosis and/or during a normal menstrual cycle. In some embodiments, the kit includes at least two binding agents specific for a differentially expressed mRNA or protein, wherein one binding agent is not labeled and is bound to an insoluble support, and the second binding agent is detectably labeled. The binding agent(s) is present in a suitable storage medium, e.g., buffered solution, typically in a suitable container. As discussed above, a binding agent may be bound to an insoluble support.

A subject kit may further include reagents for solubilizing a macromolecule from a cell membrane, buffers, washing solutions, reagents for developing a signal (e.g., from a detectably labeled binding agent), and the like.

A subject kit may further include reagents for detecting the presence or measuring the level of other components of the biological sample, including, but not limited to, a hormone, including, but not limited to, human chorionic gonadotropin, progesterone, and the like (see, e.g., Norwitz et al. (2001) N. Engl. J. Med. 345:1400-1408); and any placental product, including, but not limited to, HLA-G (a soluble class I MHC molecule).

In some embodiments, a binding agent is a nucleic acid binding agent that specifically binds a differentially expressed mRNA. In other embodiments, a binding agent is an antibody that specifically binds a differentially expressed protein.

In some embodiments, a binding agent is attached, directly or indirectly (e.g., via a linker molecule) to a solid support for use in a diagnostic assay to determine and/or measure the presence a differentially expressed mRNA or protein in a biological sample. Attachment is generally covalent, although it need not be. Solid supports include, but are not limited to, beads (e.g., polystyrene beads, magnetic beads, and the like); plastic surfaces (e.g., polystyrene or polycarbonate multi-well plates typically used in an enzyme linked immunosorbent assay (ELISA) or radioimmunoassay (RIA), and the like); sheets, e.g., nylon, nitrocellulose, and the like, which may be in the form of test strips; and chips, e.g., SiO₂ chips such as those used in microarrays. Accordingly, in some embodiments, a subject kit comprises an assay device comprising a binding agent attached to a solid support. Generally, a solid support will also include a control binding agent that binds to a control mRNA or protein. Suitable control binding agents include, e.g., a binding agent that binds an mRNA or protein that is constitutively expressed.

In some embodiments, a binding agent is provided as an array of binding agents. One type of such reagent is an array of probe nucleic acids in which the phenotype determinative genes of interest are represented. A variety of different array formats are known in the art, with a wide variety of different probe structures, substrate compositions and attachment technologies. Representative array structures of interest include those described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In many embodiments, the arrays include probes for at least 1 of the genes listed in Table 2 and/or Table 3 and/or Table 5 and/or Table 6. In certain embodiments, the number of genes that are from Table 2 and/or Table 3 and/or Table 5 and/or Table 6 that is represented on the array is at least 5, at least 10, at least 25, at least 50, at least 75 or more, including all of the genes listed in Table 2 and/or Table 3 and/or Table 5 and/or Table 6. The subject arrays may include only those genes that are listed in Table 2 and/or Table 3 and/or Table 5 and/or Table 6 or they may include additional genes that are not listed in Table 2 and/or Table 3 and/or Table 5 and/or Table 6. Where the subject arrays include probes for such additional genes, in certain embodiments the number % of additional genes that are represented does not exceed about 50%, usually does not exceed about 25%. In many embodiments where additional “non-Table 2 and/or Table 3 and/or Table 5 and/or Table 6” genes are included, a great majority of genes in the collection are phenotype determinative genes, where by great majority is meant at least about 75%, usually at least about 80% and sometimes at least about 85, 90, 95% or higher, including embodiments where 100% of the genes in the collection are phenotype determinative genes.

Another type of binding reagent that is specifically tailored for generating expression profiles of phenotype determinative genes is a collection of gene specific primers that is designed to selectively amplify such genes. Gene specific primers and methods for using the same are described in U.S. Pat. No. 5,994,076, the disclosure of which is herein incorporated by reference. Of particular interest are collections of gene specific primers that have primers for at least 1 of the genes listed in Table 2 and/or Table 3 and/or Table 5 and/or Table 6, often a plurality of these genes, e.g., at least 2, 5, 10, 15 or more. In certain embodiments, the number of genes that are from Table 2 and/or Table 3 and/or Table 5 and/or Table 6 that have primers in the collection is at least 5, at least 10, at least 25, at least 50, at least 75 or more, including all of the genes listed in Table 2 and/or Table 3 and/or Table 5 and/or Table 6. The subject gene specific primer collections may include only those genes that are listed in Table 2 and/or Table 3 and/or Table 5 and/or Table 6, or they may include primers for additional genes that are not listed in Table 2 and/or Table 3 and/or Table 5 and/or Table 6. Where the subject gene specific primer collections include primers for such additional genes, in certain embodiments the number % of additional genes that are represented does not exceed about 50%, usually does not exceed about 25%. In many embodiments where additional “non-Table 2 and/or Table 3 and/or Table 5 and/or Table 6” genes are included, a great majority of genes in the collection are phenotype determinative genes, where by great majority is meant at least about 75%, usually at least about 80% and sometimes at least about 85, 90, 95% or higher, including embodiments where 100% of the genes in the collection are phenotype determinative genes.

The kits of the subject invention may include the above described arrays and/or gene specific primer collections. The kits may further include one or more additional reagents employed in the various methods, such as primers for generating target nucleic acids, dNTPs and/or rNTPs, which may be either premixed or separate, one or more uniquely labeled dNTPs and/or rNTPs, such as biotinylated or Cy3 or Cy5 tagged dNTPs, gold or silver particles with different scattering spectra, or other post synthesis labeling reagent, such as chemically active derivatives of fluorescent dyes, enzymes, such as reverse transcriptases, DNA polymerases, RNA polymerases, and the like, various buffer mediums, e.g. hybridization and washing buffers, prefabricated probe arrays, labeled probe purification reagents and components, like spin columns, etc., signal generation and detection reagents, e.g. streptavidin-alkaline phosphatase conjugate, chemifluorescent or chemiluminescent substrate, and the like.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, compact disc (CD), etc., on which the information has been recorded. The information may be recorded on a digital versatile disk (DVD), audio cassette, video cassette, or other recording media. Yet another means that may be present is a website address which may be used via the internet to access the information at a removed site. Any convenient means may be present in the kits.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

### Genes Differentially Regulated in the Window of Implantation

Materials and Methods

Tissue Specimens and Cell Culture

Tissues. Endometrial biopsies were obtained from normally cycling women. A total of 28 biopsy samples were obtained from two time points of the menstrual cycle and used in this study: 10 in the late proliferative phase (peak circulating estradiol levels; cycle days 8-10), and 18 during the window of implantation [mid-secretory phase (peak estradiol and progesterone)] which were timed to the LH surge (LH+8 to LH+10, where LH=0 is the day of the LH surge). Timing to the LH surge assured sampling during the window of implantation. Of the 28 biopsies, 11 (4 in late proliferative phase and 7 window of implantation), were used for microarray studies, 5 secretory specimens were used exclusively for cell isolation and culture, and 12 were used for Northern analysis and RT-PCR validation. Different samples were used for the microarrays and the validation studies. Subjects ranged in age between 28-39 years of age, had regular menstrual cycles (26-35 days), were documented not to be pregnant, and had no history of endometriosis. Endometrial biopsies were performed with Pipelle catheters under sterile conditions, from the uterine fundus. A portion of each sample was processed for histologic confirmation, and the remainder was processed for cell culture or immediately frozen in liquid nitrogen for subsequent RNA isolation. Cycle stages for all specimens (proliferative and mid-secretory) were histologically confirmed independently by three observers: LCG, BAL, and an independent pathologist.

Cell Culture. Five mid-secretory specimens were used for cell isolation and culture for this study. Tissue was subjected to collagenase (Sigma, Mo.) digestion, and stromal cells were separated from epithelium. Initially, stromal cells were centrifuged and the resulting pellet was resuspended in DMEM/10% fetal bovine serum (FBS). The cells were then pre-plated in 10 cm standard culture plates in DMEM/F12 media for 1 hr at 37° C., and the media was then replaced with DMEM/10%FBS. Glands retained on the filter were backwashed into sterile tubes, washed with phosphate buffered saline (PBS) three times, centrifuged and resuspended in MCDB-105. Endometrial stromal cells were plated and passaged in standard tissue culture plates at a density of 2-3×10⁵/10 cm plate and cultured in phenol-red-free, high-glucose DMEM/MCDB-105 medium with 10% charcoal-stripped FBS, insulin (5 μg/ml), gentamicin, penicillin and streptomycin. Stromal cells were used at passages 2-6 for these studies. Endometrial epithelial cells were plated in two chamber collagen type I-coated chamber slides (Co-star, Cambridge, Mass.) and cultured in MEMα with 10% charcoal-stripped FBS at 37° C. in 9% CO₂ for up to one week. Purity was established by vimentin and cytokeratin immunostaining. The culture medium was renewed every two days, and the cells were harvested for RNA analysis at the end of the culture period.

Gene Expression Profiling

RNA Preparation/Target Preparation/Array Hybridization and Scanning. For microarray analysis, N=4 late proliferative phase samples and N=7 window of implantation samples were used. Each endometrial biopsy sample was processed individually for microarray hybridization (samples were not pooled) following the Affymetrix (Affymetrix, Santa Clara, Calif.) protocol. Poly(A)⁺-RNA was initially isolated from the tissue samples using Oligotex® Direct mRNA isolation kits (Qiagen, Valencia, Calif.), following the manufacturer's instructions. Specimens (120-260 mg) yielded between 1-8 μg poly(A)⁺-RNA and the purity of isolated mRNAs was evaluated spectrophotometrically by the A260/A280 ratio. A T7-(dT)₂₄ oligo-primer was used for double stranded cDNA synthesis by the Superscript Choice System (GIBCO-BRL). In vitro transcription was subsequently carried out with Enzo BioArray High Yield RNA T7 Transcript Labeling Kits (ENZO, Farmingdale, N.Y.). Additional cRNA clean-up was performed using RNeasy spin columns (Qiagen), prior to chemical fragmentation with 5×fragmentation buffer (200 mM Tris, pH 8.1, 500 mM KOAc, 150 mM MgOAc). After chemical fragmentation, biotinylated cRNAs were mixed with controls and were hybridized to Affymetrix Genechip Hu95A oligonucleotide microarrays [corresponding to 12,686 human genes and expressed sequence tags (ESTs)] on an Affymetrix fluidics station at the Stanford University School of Medicine Protein and Nucleic Acid (PAN) Facility. Fluorescent labeling and laser confocal scanning were conducted in the PAN Facility and generated the data for analysis.

TABLE 1

Oligonucleotide primers with predicted respective PCR product sizes

| | | |
|---|---|---|
| Gene | Sense primers | Antisense primers bp |

| | | |
|---|---|---|
| IGFBP-1 | 5′-ACTCTGCTGGTGCGTCTAC-3′; | 5′-TTAACCGTCCTCCTTCAAAC-3′ |
| | SEQ ID NQ:01 | SEQ ID NO:02; (499 bp PCR product) |
| Glycodelin | 5′-AAGTTGGCAGGGACCTGGCACTC-3′; | 5′-ACGGCACGGCTCTTCCATCTGTT-3′ |
| | SEQ ID NO:03 | SEQ ID NO:04; (420 bp PCR product) |
| CPE-1 R | 5′-TACTCCGCCAAGTATTCTG-3′; | 5′-ATTACAGTGATGAATAGCTGTT-3′; |
| | SEQ ID NO:05 | SEQ ID NO:06; (900 bp PCR product) |
| Dkk-1 | 5′-AGGCGTGCAAATCTGTCTCG-3′; | 5′-TGCATTTGGATAGCTGGTTTAGT-3′; |
| | SEQ ID NO:07 | SEQ ID NO:08 (502 bp PCR product) |
| GABAA R subunit | 5′-GCTGGGGCTATGATGGAAATG-3′; | 5′-CTAGCAAGGCCCCAAACACAAAG-3′; |
| | SEQ ID NO:09 | SEQ ID NO:10; (429 bp PCR product) |
| Mammaglobin | 5′-AGTTGCTGATGGTCCTCATG-3′; | 5′-AGAAGGTGTGGTTTGCAGC-3′; |
| | SEQ ID NO:11 | SEQ ID NO:12; (358 bp PCR product) |
| Apolipoprotein D | 5′-AAAAGCTCCAGGTCCCTTC-3′; | 5′-AGGGTTTCTTGCCAAGATCC-3′; |
| | SEQ ID NO:13 | SEQ ID NO:14; (498 bp PCR product) |
| PGRMC-1 | 5′-CTTCCTGCTCTACAAGATCG-3′; | 5′-CCTCATCTGAGTAGACAGTG-3′; |
| | SEQ ID NO:15 | SEQ ID NO:16; (408 bp PCR product) |
| FrpH E | 5′-CCGTGCTGCGCTTCTTCTTCTGTG-3′; | 5′-GCGGGACTTGAGTTCGAGGGATGG-3′; |
| | SEQ ID NQ:17 | SEQ ID NO:18; (461 bp PCR product) |
| Matrilysin | 5′-GTCTCAATAGGAAAGAGAAG-3′; | 5′-TGAATAAGACACAGTCACAC-3′; |
| | SEQ ID NO:19 | SEQ ID NO:20; (230 bp PCR product) |
| ITE | 5′-TTGCTGTCCTGCAGCTCTG-3′; | 5′-CAGGCTCCAGATATGAAC-3′; |
| | SEQ ID NO:21 | SEQ ID NO:22; (322 bp PCR product) |
| GAPDH | 5′-CACAGTCCATGCCATCACTGC-3′; | 5′-GGTCTACATGGGAACTGTGAG-3′ |
| | SEQ ID NO:23 | SEQ ID NO:24; (609 bp PCR product) |

Data Analysis. One of the most critical steps in microarray profiling experiments is accurate assessment of the expression ratios between the sample and the reference, because most subsequent analyses depend on the accuracy of these ratios. The observed signal is comprised of the true expression level with noise due to background and noise due to experimental variations from the probe preparation and hybridization efficiency. Due to variations in the hybridization and scanning processes, several approaches for data analysis have been devised to compensate for these differences.

Two major steps are: 1) to eliminate weak expressions that are statistically too close to the background estimate to avoid the detrimental effects on the ratios, and 2) to adjust the expression of each gene by the over-all expression of signals on a specific chip. In the current study the data were analyzed with GeneChip® Analysis Suite v4.01 (Affymetrix), GeneSpring v4.0.4 (Silicon Genetics), and Microsoft Excel/Mac2001 software. Expression profile data were first prepared using GeneChip Microarray Analysis Suite® and subsequently exported to GeneSpring for further analysis. The GeneSpring v4.0.4 software allows rank-sum normalization and statistical analysis. Initially, within each hybridization, the 50th percentile of all measurements was used as a positive control, and each measurement for each gene was divided by this control. The bottom tenth percentile was used for background subtraction.

Between different hybridization outputs/arrays, each gene was normalized to itself by making a synthetic positive control for that gene comprised of the median of the gene's expression values over all samples of an experimental group, and dividing the measurements for that gene by this positive control, as per the manufacturer's instructions. Mean values were then calculated among individual experimental groups for each gene probe-set, and between-group “fold-change” ratios [i.e., window of implantation (N=4): late proliferative phase (N=7) ratios] were derived. A difference of 2-fold was applied to select up-regulated and down-regulated genes.

Since the data were not normally distributed, non-parametric testing was also conducted using the Mann-Whitney U test to calculate p-values, and applying p<0.05 to assign statistical significance between the two groups. To assess chip-to-chip variability, preliminary experiments were conducted in which RNA from one tissue sample was subjected to two independent hybridizations. Less than 2.7% of the total genes on the array showed more than 3-fold variation, providing a greater than 95% confidence level, consistent with the manufacturer's claims for chip-to-chip variability.

Validation of Gene Expression Data. Reverse transcription-polymerase chain reaction (RT-PCR). Genes of different expression fold changes were randomly selected for validation by RT-PCR and/or Northern analyses. Total RNA from cultured endometrial epithelial cells, stromal cells or whole endometrial tissue was isolated using Trizol (Gibco/BRL, MD) protocol, then treated with DNase (Qiagen) and purified by RNeasy Spin Columns (Qiagen). Reverse transcription was first performed with Omniscript kit (Qiagen) for 1 h at 37° C., followed by PCR in a 50 μl reaction volume with Taq polymerase (Qiagen) and specific primer pairs using the Eppendorf Mastercycler Gradient. The amplification cycle consisted of a hot start at 94° C. for 2 min followed by 35 cycles of denaturation at 94° C. for 1 min, annealing at 58° C. for 1 min and extension at 72° C. for 1 min. Specific primer pairs (Table I) were synthesized by the PAN Facility, Stanford University School of Medicine, and were used at 25 pmol per reaction. Sequences were derived from public databases, and all PCR products were confirmed by the Stanford PAN Sequencing Facility. Subcloning by TA cloning into pGEM Teasy (Promega, Madison, Wis.) or pDrive Cloning Vector (Qiagen) were performed to generate specific probes for Northern analyses.

Northern Analysis. Twelve endometrial biopsy samples were used for these studies, 6 from the late proliferative phase and 6 during the window of implantation. Total RNAs (10-20 μg) were electrophoresed on 1% formaldehyde agarose gels and transferred to Nylon membranes for Northern analyses. Specific P³²-labeled cDNA probes, ranging 400-900 bp, were generated using Ready-to-Go random primer kit (Pharmacia Biotech, Peapack, N.J.) and ³²αP-dCTP (NEN Life Science Products, Boston, Mass.). Membranes were prehybridized at 68° C. for 30 min in ExpressHyb buffer (Clontech, Palo Alto, Calif.) and hybridization carried out for another hour at 68° C. using ExpressHyb buffer containing 1-2×10⁶ cpm/ml of labeled probe. Washing was subsequently carried out according to the manufacturers' instructions. Membranes were exposed to Kodak MS X-ray films, and densitometry performed with Bio-Rad GS-710 Imaging Densitometer (Bio-Rad, Hercules, Calif.) and analyzed by its accompanied software Quantity One, v.4.0.2. GAPDH mRNA intensities were used for normalization prior to comparison. Mean values of relative expression intensities from different blots were used for final data presentation. Stripping and reprobing were performed using the same membranes.

Results

Data Analysis. The data were analyzed with GeneChip® Analysis Suite v4.01, GeneSpring v4.0.4, and Microsoft Excel/Mac2001 software, as described in Materials and Methods. A scatter plot of the normalized data for all genes and all experiments for samples in the proliferative phase and the secretory phase (window of implantation), showed that the data are not normally distributed. Fold-change ratios between groups (i.e., window of implantation: late proliferative phase ratios) were subsequently derived, and a difference of 2-fold, a generally adopted fold-change difference for oligonucleotide microarray profile analysis, was applied to select up-regulated and down-regulated genes.

Nonparametric testing was further applied, using a P-value of 0.05 to identify statistical significance between the two groups. With this strategy, we identified, during the window of implantation, 156 genes that were significantly upregulated, of which 40 were ESTs, and 377 genes that were significantly down-regulated, of which 153 were ESTs. Table 2 and Table 3 show, in descending order, respectively, the fold increase and fold decrease, the P-values (P<0.05), and the GenBank accession numbers for the 116 specifically up-regulated genes (Table 2) and the 224 down-regulated genes (Table 3) in the window of implantation in human endometrium, compared to the late proliferative phase, according to clustering assignments.

TABLE 2

| | | | |
|---|---|---|---|
| Families/ | | | |
| GenBank | | | |
| Accession No. | Fold Up | p-value | Description (N = 156) |

cholesterol transprt/trafficking

| | | | |
|---|---|---|---|
| M12529 | 100.0 | 0.013 | apolipoprotein-E |
| J02611 | 5.6 | 0.0013 | apolipoprotein-D |

prostaglandin biosynthesis

| | | | |
|---|---|---|---|
| M22430 | 18.2 | 0.0300 | RASF-A PLA2 (phospholipase A2) |
| U19487 | 3.6 | 0.0300 | prostaglandin E2 receptor |

carbohydrate/glycoprotein synthesis

| | | | |
|---|---|---|---|
| AB009598 | 15.6 | 0.03 | glucuronyltransferase I |
| AB014679 | 6.4 | 0.0066 | N-acetylglucosamine-6-O-sulfotransferase (GlcNAc6ST) |

secretory proteins

| | | | |
|---|---|---|---|
| M61886 | 14.6 | 0.0272 | pregnancy-associated endometrial alpha2-globulin |
| | | | (glycodelin) |
| U33147 | 12.4 | 0.0255 | mammaglobin |
| AB020315 | 12.1 | 0.0057 | Dickkopf-1 (hdkk-1) |
| M31452 | 7.0 | 0.0272 | proline-rich protein (PRP) |
| M57730 | 4.9 | 0.0057 | B61 |
| X16302 | 2.7 | 0.0130 | insulin-like growth factor binding protein (IGFBP-2) |
| M93311 | 2.4 | 0.0049 | metallothionein-III |
| AB000584 | 2.4 | 0.0057 | TGF-beta superfamily protein |

cell cycle

| | | | |
|---|---|---|---|
| M69199 | 9.2 | 0.0184 | G0S2 protein |
| M14752 | 6.4 | 0.0300 | c-abl |
| M60974 | 3.9 | 0.0057 | growth arrest and DNA-damage-inducible protein |
| | | | (gadd45) |
| AF002697 | 2.2 | 0.0130 | E1B 19K/Bcl-2-binding protein Nip3 |
| U66469 | 2.0 | 0.0418 | cell growth regulator CGR19 |

proteases/peptidases

| | | | |
|---|---|---|---|
| M17016 | 9.0 | 0.0130 | Serine protease-like protein |
| M30474 | 5.2 | 0.0343 | gamma-glutamyl transpeptidase type II |
| L12468 | 4.0 | 0.0279 | aminopeptidase A |
| AL008726 | 2.5 | 0.0013 | Lysosomal protective protein precursor, cathepsin A, |
| | | | carboxypeptidase C |
| nitric oxide synthesis | 8.3 | 0.0057 | arginase type II |
| U82256 | | | |

extracellular matrix/cell adhesion molecules

| | | | |
|---|---|---|---|
| J04765 | 8.1 | 0.0013 | osteopontin |
| U17760 | 4.1 | 0.0017 | laminin S B3 chain |
| M61916 | 2.6 | 0.0184 | laminin B1 chain |

Neuromodulators/synthesis/receptors

| | | | |
|---|---|---|---|
| M68840 | 7.5 | 0.0013 | monoamine oxidase A (MAOA) |
| U95367 | 2.6 | 0.0437 | GABA-A receptor pi subunit |

immune modulators/cytokines

| | | | |
|---|---|---|---|
| L41268 | 7.2 | 0.0082 | natural killer-associated transcript 2 (NKAT2) |
| M84526 | 6.7 | 0.0272 | adipsin/complement factor D |
| M31516 | 5.9 | 0.0013 | Decay-accelerating factor |
| AF031167 | 5.9 | 0.0013 | interleukin 15 precursor (IL-15) |
| D63789 | 4.5 | 0.0300 | SCM-1 beta precursor (lymphotactin) |
| M85276 | 4.0 | 0.0437 | NKG5 NK & T-cell specific gene |
| U14407 | 3.7 | 0.0300 | interleukin 15 (IL15) |
| M34455 | 3.7 | 0.0049 | interferon-gamma-inducible indoleamine 2,3-dioxygenase |
| | | | (IDO) |
| U31628 | 3.3 | 0.0066 | interleukin-15 receptor alpha chain precursor (IL15RA) |
| AC006293 | 2.9 | 0.0082 | chromosome 19, cosmid F15658 |
| D87002 | 2.4 | 0.0130 | immunoglobulin lambda gene locus |
| L09708 | 2.1 | 0.0279 | complement component 2 (C2) |
| M14058 | 2.0 | 0.0130 | complement C1r |

Detoxification

| | | | |
|---|---|---|---|
| J03910 | 5.9 | 0.0013 | metallothionein-IG (MTIG) |
| M10943 | 3.8 | 0.0049 | metallothionein-If |
| R93527 | 3.6 | 0.0049 | Homo sapiens cDNA similar to metallothionein |
| M13485 | 3.5 | 0.0013 | metallothionein I-B |
| H68340 | 3.5 | 0.0049 | Homo sapiens cDNA similar to metallothionein-If |
| K01383 | 3.0 | 0.0279 | metallothionein-I-A |
| X71973 | 2.9 | 0.0130 | phospholipid hydroperoxide glutathione peroxidase |

structural/cytoskeletal proteins

| | | | |
|---|---|---|---|
| M88338 | 5.2 | 0.0418 | Serum constituent protein (MSE55) |
| M34175 | 4.3 | 0.0212 | beta adaptin |
| M19267 | 3.7 | 0.0300 | tropomyosin |
| X06956 | 3.4 | 0.0082 | Alpha-tubulin |

phospholipid binding proteins

| | | | |
|---|---|---|---|
| D28364 | 4.7 | 0.0300 | Annexin II |
| M82809 | 2.2 | 0.0279 | Annexin IV (ANX4) |

cell surface proteins/receptors

| | | | |
|---|---|---|---|
| L78207 | 4.3 | 0.0013 | sulfonylurea receptor (SUR1)(K⁺-channel) |
| U11863 | 3.4 | 0.0418 | HP-DAO2 diamine oxidase, copper/topa quinone |
| | | | containing mRNA |
| J03779 | 2.7 | 0.0184 | common acute lymphoblastic leukemia antigen (CALLA) |
| D50683 | 2.6 | 0.0437 | TGF-beta II-R alpha |
| X97324 | 2.1 | 0.0272 | adipophilin |

Transporters

| | | | |
|---|---|---|---|
| AB000712 | 3.9 | 0.0272 | HCPE-R (Clostridia Perfringens Enterotoxin receptor-1) |
| U81800 | 3.4 | 0.0057 | monocarboxylate transporter (MCT3) |
| U36341 | 2.9 | 0.0255 | creatine transporter (SLC6A8) |
| AJ131182 | 2.5 | 0.0130 | Epsilon COP |
| AB000714 | 2.2 | 0.0057 | HRVP1 (splice variant of CPE-R) |
| X57522 | 2.1 | 0.0437 | RING4 |

transcription factors

| | | | |
|---|---|---|---|
| J04102 | 3.9 | 0.0255 | erythroblastosis virus oncogene homolog 2 (ets-2) |
| V00568 | 3.1 | 0.0437 | c-myc |
| U51127 | 2.8 | 0.0300 | interferon regulatory factor 5 (Humirf5) |
| AL022726 | 2.8 | 0.0130 | ID4 Helix-loop-helix DNA binding protein |
| L32164 | 2.4 | 0.0117 | zinc finger protein |

signal transduction

| | | | |
|---|---|---|---|
| Y10032 | 3.6 | 0.0066 | putative serine/threonine protein kinase |
| D87953 | 3.5 | 0.0013 | RTP |
| X69550 | 2.9 | 0.0272 | rho GDP-dissociation inhibitor 1 |
| L76200 | 2.8 | 0.0130 | guanylate kinase (GUK1) |
| U67156 | 2.7 | 0.0013 | mitogen-activated kinase kinase kinase 5 (MAPKKK5) |
| D38305 | 2.5 | 0.0130 | Tob |
| tigr:HG162-HT3165 | 2.4 | 0.0066 | Tyrosine Kinase, Receptor Axi, Alt. Splice 2 |
| M54915 | 2.1 | 0.0013 | h-pim-1 protein (h-pim-1) |
| L12535 | 2.1 | 0.0279 | RSU-1/RSP-1 |

other cellular functions

| | | | |
|---|---|---|---|
| U07919 | 7.3 | 0.0057 | aldehyde dehydrogenase 6 |
| U12778 | 3.7 | 0.0130 | acyl-CoA dehydrogenase |
| M94856 | 3.5 | 0.0130 | fatty acid binding protein homologue (PA-FABP) |
| U80184 | 3.4 | 0.0272 | FLII |
| U09196 | 3.4 | 0.0013 | 1.1 kb mRNA upregulated in retinoic acid treated HL-60 |
| | | | neutrophilic cells |
| AF042800 | 3.4 | 0.0300 | suppressor of white apricot homolog 2 (SWAP2) |
| D83198 | 3.3 | 0.0013 | mRNA expressed in thyroid gland |
| X79882 | 3.2 | 0.0057 | Lrp |
| M62896 | 3.2 | 0.0057 | lipocortin (LIP) 2 pseudogene mRNA |
| D38047 | 3.0 | 0.0013 | 26S proteasome subunit p31 |
| U90551 | 3.0 | 0.0057 | histone 2A-like protein (H2A/l) |
| AJ223352 | 2.8 | 0.0130 | histone H2B |
| L38928 | 2.8 | 0.0437 | 5,10-methenyltetrahydrofolate synthetase |
| L33799 | 2.7 | 0.0130 | procollagen C-proteinase enhancer protein (PCOLCE) |
| U20938 | 2.7 | 0.0255 | lymphocyte dihydropyrimidine dehydrogenase |
| S72370 | 2.6 | 0.0066 | pyruvate carboxylase |
| X00737 | 2.6 | 0.0437 | purine nucleoside phosphorylase |
| X59960 | 2.6 | 0.0279 | sphingomyelinase |
| X15573 | 2.6 | 0.0300 | liver-type 1-phosphofructokinase (PFKL) |
| D26535 | 2.5 | 0.0300 | dihydrolipoamide succinyltransferase |
| U02556 | 2.6 | 0.0279 | RP3 |
| U78190 | 2.5 | 0.0300 | GTP cyclohydrolase I feedback regulatory protein (GFRP) |
| AF090421 | 2.5 | 0.0255 | ribosome S6 protein kinase |
| AF054825 | 2.4 | 0.0437 | VAMPS |
| J04444 | 2.4 | 0.0130 | cytochrome c-1 |
| X02152 | 2.3 | 0.0049 | lactate dehydrogenase-A (LDH-A) |
| M61832 | 2.3 | 0.0437 | S-adenosylhomocysteifle hydrolase (AHCY) |
| U61263 | 2.2 | 0.0130 | acetolactate synthase homolog |
| Z80779 | 2.2 | 0.0388 | H2B/g |
| X13973 | 2.2 | 0.0279 | ribonuclease/angiogenin inhibitor (RAI) |
| AF000573 | 2.2 | 0.0437 | homogentisate 1,2-dioxygenase |
| X93086 | 2.1 | 0.0212 | biliverdin IX alpha reductase |
| AF042386 | 2.2 | 0.0255 | cyclophilin-33B (CYP-33) |
| AF020736 | 2.0 | 0.0130 | ATPase homolog |

| | |
|---|---|
| EST's/Unknown | N = 40 |
| function | |

TABLE 3

| | | | |
|---|---|---|---|
| Families/GenBank | | | |
| Accession No. | Fold Down | p-value | Description (N = 377) |

secretory proteins

| | | | |
|---|---|---|---|
| L08044 | 49.8 | 0.0418 | intestinal trefoil factor |
| AF026692 | 19.8 | 0.0017 | frizzled related rotein frpHE |
| AF056087 | 6.3 | 0.0013 | secreted frizzled related protein FRP |
| AB000220 | 5.8 | 0.0047 | semaphorin E |
| X78947 | 2.9 | 0.0279 | connective tissue growth factor |
| U38276 | 2.6 | 0.0130 | semaphorin III family homolog |
| AF020044 | 2.2 | 0.0130 | lymphocyte secreted C-type lectin precursor |

proteases

| | | | |
|---|---|---|---|
| L22524 | 24.1 | 0.0082 | matrilysin |
| M96859 | 10.8 | 0.0213 | dipeptidyl aminopeptidase like protein |
| X51405 | 9.7 | 0.0117 | carboxypeptidase E |
| AF071748 | 3.1 | 0.0117 | cathepsin F (CATSF) |

signal transduction

| | | | |
|---|---|---|---|
| L15388 | 23.5 | 0.0213 | G protein-coupled receptor kinase (GRK5) |
| M29551 | 7.6 | 0.0464 | calcineurin A2 |
| AB007972 | 5.3 | 0.0130 | chromosome 1 specific transcript KIAA0503 |
| L06139 | 5.1 | 0.0279 | receptor protein-tyrosine kinase (TEK) |
| U31384 | 4.7 | 0.0212 | G protein gamma-11 subunit |
| L07592 | 3.9 | 0.0213 | peroxisome proliferator activated receptor |
| S62539 | 3.5 | 0.0013 | insulin receptor substrate-1 |
| U02390 | 3.4 | 0.0274 | adenylyl cyclase-associated protein homolog CAP2 |
| | | | (CAP2) |
| D87116 | 3.4 | 0.0212 | MAP kinase kinase 3b |
| AB015019 | 3.2 | 0.0274 | BAP2-alpha |
| AB009356 | 3.2 | 0.0049 | TGF-beta activated kinase 1a |
| U61167 | 3.1 | 0.0130 | SH3 domain-containing protein SH3P18 |
| AF015254 | 3.1 | 0.0117 | serine/threonine kinase (STK-1) |
| U59863 | 2.9 | 0.0212 | TRAF-interacting protein 1-TRAF |
| U36764 | 2.8 | 0.0300 | TGF-beta receptor interacting protein 1 |
| D50863 | 2.6 | 0.0017 | TESK1 |
| L33881 | 2.5 | 0.0212 | protein kinase C iota isoform |
| U59912 | 2.4 | 0.0049 | Smad1 |
| Y18046 | 2.4 | 0.0117 | FOP (FGFR1 oncogene partner) |
| S59184 | 2.4 | 0.0212 | RYK = related to receptor tyrosine kinase |
| AF042081 | 2.3 | 0.0279 | SH3 domain binding glutamic acid-rich-like protein |
| X56468 | 2.2 | 0.0049 | mRNA for 14.3.3 protein, a protein kinase regulator |
| U94905 | 2.2 | 0.0013 | diacylglycerol kinase zeta |
| D10522 | 2.2 | 0.0130 | 80K-L protein |
| U37139 | 2.2 | 0.0025 | beta 3-endonexin |
| L36870 | 2.2 | 0.0386 | MAP kinase kinase 4 (MKK4) |
| U02570 | 2.2 | 0.0279 | CDC42 GTPase-activating protein |
| U85245 | 2.1 | 0.0049 | phosphatidylinositol-4-phosphate 5-kinase type II |
| | | | beta |
| X02596 | 2.0 | 0.0013 | bcr (breakpoint cluster region) gene in Philadelphia |
| | | | chromosome |

cell surface proteins/receptors

| | | | |
|---|---|---|---|
| D10925 | 11.3 | 0.0082 | HM145 |
| L78132 | 4.8 | 0.0133 | prostate carcinoma tumor antigen (pcta-1) |
| AB011542 | 3.5 | 0.0177 | MEGF9 |
| M34641 | 3.4 | 0.0013 | fibroblast growth factor (FGF) receptor-1 |
| M87770 | 3.2 | 0.0212 | fibroblast growth factor receptor (K-sam) |
| U09278 | 3.2 | 0.0130 | fibroblast activation protein |
| AB015633 | 3.0 | 0.0017 | type II membrane protein |
| X83425 | 2.7 | 0.0388 | Lutheran blood group glycoprotein |
| Y00264 | 2.6 | 0.0130 | amyloid A4 precursor |
| L20852 | 2.2 | 0.0212 | leukemia virus receptor-2 (GLVR2) |

extracellular matrix/cell adhesion molecules

| | | | |
|---|---|---|---|
| M92642 | 11.2 | 0.0013 | alpha-1 type XVI collagen (COL16A1) |
| AL049946 | 10.1 | 0.0017 | DKFZp564l1922 |
| M34064 | 6.0 | 0.0013 | human N-cadherin |
| U69263 | 5.6 | 0.0117 | matrilin-2 precursor |
| J04599 | 4.2 | 0.0013 | hPGI mRNA encoding bone small proteoglycan I |
| | | | (biglycan) |
| X78565 | 3.9 | 0.0130 | tenascin-C |
| U19718 | 3.0 | 0.0066 | microfibril-associated glycoprotein (MFAP2) |
| D13666 | 2.8 | 0.0117 | osteoblast specific factor-2 (OSF-2os) |
| X53002 | 2.4 | 0.0049 | integrin beta-5 subunit |
| X53586 | 2.3 | 0.0013 | integrin alpha 6 |
| X17042 | 2.1 | 0.0279 | hematopoetic proteoglycan core protein |

transcription factors

| | | | |
|---|---|---|---|
| D89377 | 9.0 | 0.0213 | MSX-2 |
| L11672 | 7.2 | 0.0343 | Kruppel related zinc finger protein (HTF10) |
| M21535 | 6.1 | 0.0386 | erg protein (ets-related gene) |
| V01512 | 4.9 | 0.0464 | oncogene c-fos |
| U09848 | 4.8 | 0.0343 | zinc finger protein (ZNF139) |
| M68891 | 4.0 | 0.0418 | GATA-binding protein (GATA2) |
| AJ222700 | 4.0 | 0.0049 | TSC-22 protein |
| X62534 | 3.8 | 0.0130 | HMG-2 |
| AF003540 | 3.1 | 0.0343 | Kruppel family zinc finger protein (znfp104) |
| X07384 | 3.0 | 0.0117 | GLI protein |
| M31523 | 3.0 | 0.0184 | transcription factor (E2A) |
| L13689 | 3.0 | 0.0279 | proto-oncogene (BMI-1) |
| AF045451 | 2.9 | 0.0049 | transcriptional regulatory protein p54 |
| D63874 | 2.8 | 0.0017 | HMG-1 |
| AL096880 | 2.8 | 0.0049 | mRNA containing zinc finger C2H2 type domains |
| AC004774 | 2.8 | 0.0057 | BAC clone RG300E22 |
| X59871 | 2.8 | 0.0213 | T cell factor 1 (TCF-1, splice form C) |
| M97676 | 2.7 | 0.0388 | homeobox protein (HOX7) |
| L19314 | 2.7 | 0.0047 | HRY |
| X84373 | 2.7 | 0.0130 | nuclear factor RIP140 |
| X53390 | 2.6 | 0.0025 | upstream binding factor (hUBF) |
| X17360 | 2.5 | 0.0279 | HOX 5.1 |
| AF071309 | 2.5 | 0.0013 | OPA-containing protein |
| AJ223321 | 2.5 | 0.0017 | RP58 |
| U80760 | 2.4 | 0.0130 | CAGH1 alternate open reading frame |
| D28118 | 2.4 | 0.0464 | DB1 |
| M16937 | 2.3 | 0.0057 | homeobox c1 protein |
| U31814 | 2.3 | 0.0049 | transcriptional regulator homolog RPD3 |
| AF104913 | 2.3 | 0.0300 | eukaryotic protein synthesis initiation factor |
| D13969 | 2.3 | 0.0049 | Mel-18 protein |
| AL031668 | 2.3 | 0.0049 | EIF2S2 [eukaryotic translation initiation factor 2, |
| | | | subunit 2 (β, 38kD)] |
| X59268 | 2.3 | 0.0117 | transcription factor IIB |
| AF031383 | 2.2 | 0.0049 | hMed7 (MED7) |
| AB006572 | 2.2 | 0.0130 | RMP mRNA for RPB5 mediating protein |
| M27691 | 2.1 | 0.0212 | transactivator protein (CREB) |
| X72889 | 2.1 | 0.0130 | hbrm |
| D85939 | 2.1 | 0.0274 | p97 homologous protein |
| M62831 | 2.1 | 0.0130 | transcription factor ETR101 |
| X95525 | 2.1 | 0.0013 | TAFII100 protein |

apoptosis/inhibitors

| | | | |
|---|---|---|---|
| AF001294 | 5.6 | 0.0117 | IPL |
| AF036956 | 4.4 | 0.0047 | neuroblastoma apoptosis-related RNA binding |
| | | | protein (NAPOR-1) |
| M96954 | 3.0 | 0.0213 | nucleolysin |
| M77142 | 2.7 | 0.0130 | polyadenylate binding protein (TIA-1) |
| AF005775 | 2.3 | 0.0212 | caspase-like apoptosis regulatory protein 2 (clarp) |
| AF016266 | 2.2 | 0.0013 | TRAIL receptor 2 |
| M59465 | 2.0 | 0.0464 | tumor necrosis factor alpha inducible protein A20 |

immune modulators/receptors

| | | | |
|---|---|---|---|
| M83664 | 4.7 | 0.0049 | MHC class II lymphocyte antigen (HLA-DP) beta |
| | | | chain |
| M60028 | 4.6 | 0.0017 | MHC class II HLA-DQ-beta (DQB1,DQw9) |
| X94232 | 3.5 | 0.0386 | T-cell activation protein |
| J00194 | 2.9 | 0.0130 | HLA-dr antigen alpha-chain |
| M24594 | 2.6 | 0.0213 | interferon-inducible 56Kd protein |

vasoactive substances

| | | | |
|---|---|---|---|
| J05081 | 4.7 | 0.0418 | endothelin 3 (EDN3) |
| AF022375 | 3.4 | 0.0279 | vascular endothelial growth factor |

cell cycle

| | | | |
|---|---|---|---|
| X77494 | 4.1 | 0.0279 | MSSP-2 |
| AF017790 | 4.1 | 0.0117 | retinoblastoma-associated protein HEC |
| AF059617 | 3.5 | 0.0212 | serum-inducible kinase |
| M68520 | 3.4 | 0.0049 | cdc2-related protein kinase |
| AB000449 | 3.2 | 0.0418 | VRK1 |
| D38073 | 2.7 | 0.0343 | hRlf beta subunit (p102 protein) |
| U37359 | 2.6 | 0.0213 | MRE11 homologue hMre11 |
| M25753 | 2.5 | 0.0213 | cyclin B |
| L20046 | 2.5 | 0.0133 | ERCC5 excision repair protein |
| U50535 | 2.4 | 0.0300 | BRCA2 |
| X59798 | 2.4 | 0.0013 | PRAD1 mRNA for cyclin |
| L78833 | 2.2 | 0.0274 | BRCA1, Rho7 and vatl genes |

structural/cytoskeletal proteins

| | | | |
|---|---|---|---|
| L10678 | 3.0 | 0.0049 | profilin II |
| AF027299 | 2.6 | 0.0279 | protein 4.1-G |
| S78296 | 2.1 | 0.0418 | neurofilament-66 |
| U03057 | 2.1 | 0.0013 | actin bundling protein (HSN) |

transport proteins

| | | | |
|---|---|---|---|
| L04569 | 2.8 | 0.0213 | L-type voltage-dependent calcium channel a1 |
| | | | subunit (hHT) |
| U83993 | 2.5 | 0.0057 | P2X4 purinoreceptor |
| U07139 | 2.0 | 0.0130 | voltage-gated calcium channel beta subunit |

ion binding proteins

| | | | |
|---|---|---|---|
| X72964 | 2.5 | 0.0013 | caltractin |
| AF070616 | 2.1 | 0.0049 | BDP-1 protein |
| M81637 | 2.1 | 0.0388 | grancalcin |
| U29091 | 2.0 | 0.0279 | selenium-binding protein (hSBP) |

steroid hormone action

| | | | |
|---|---|---|---|
| Y12711 | 2.4 | 0.0057 | putative progesterone binding protein |
| AJ000882 | 2.1 | 0.0212 | steroid receptor coactivator 1e |
| neuromodulators/ | 2.2 | 0.0418 | neuronal pentraxin II (NPTX2) |
| receptors | | | |
| U29195 | | | |

Other cellular functions

| | | | |
|---|---|---|---|
| AF041210 | 7.1 | 0.0013 | midline 1 fetal kidney isoform 3 (MID1) |
| M97815 | 5.6 | 0.0274 | retinoic acid-binding protein II (CRABP-II) |
| M90656 | 5.1 | 0.0130 | gamma-glutamylcysteine synthetase (GCS) |
| U16954 | 5.1 | 0.0386 | AF1q |
| X69838 | 5.1 | 0.0082 | G9a |
| U90268 | 4.6 | 0.0418 | Krit1 |
| AJ000644 | 4.5 | 0.0279 | SPOP |
| U79299 | 4.2 | 0.0418 | neuronal olfactomedin-related ER localized protein |
| M14539 | 4.1 | 0.0025 | factor XIII subunit a |
| U57646 | 4.0 | 0.0047 | cysteine and glycine-rich protein 2 (CSRP2) |
| U03911 | 3.8 | 0.0049 | Human mutator gene (hMSH2) |
| AJ001381 | 3.7 | 0.0279 | myh-1c |
| AL031230 | 3.6 | 0.0117 | NAD+-dependent succinic semialdehyde |
| | | | dehydrogenase (SSADH) |
| U78027 | 3.5 | 0.0057 | Brutons tyrosine kinase (BTK), alpha-D |
| | | | galactosidase A (GLA), L44-like ribosomal protein |
| | | | (L44L) and FTP3 (FTP3) |
| J02683 | 3.5 | 0.0049 | ADP/ATP carrier protein |
| AC004770 | 3.3 | 0.0057 | hFEN1 |
| D89053 | 3.3 | 0.0212 | Acyl-CoA synthetase 3 |
| L35594 | 3.2 | 0.0279 | autotaxin |
| U42360 | 3.2 | 0.0279 | N33 |
| U46689 | 3.1 | 0.0013 | microsomal aldehyde dehydrogenase (ALD10) |
| U39067 | 3.1 | 0.0418 | translation initiation factor eIF3 p36 subunit |
| S71018 | 3.0 | 0.0184 | cyclophilin C |
| X96752 | 3.0 | 0.0013 | L-3-hydroxyacyl-CoA dehydrogenase |
| U90030 | 3.0 | 0.0076 | bicaudal-D (BICD) |
| S79639 | 3.0 | 0.0388 | EXT1 = putative tumor suppressor/hereditary multiple |
| | | | exostoses candidate gene |
| AF000416 | 3.0 | 0.0130 | EXT-like protein 2 (EXTL2) |
| AJ131244 | 2.9 | 0.0130 | Sec24 protein (Sec24A isoform) |
| D38076 | 2.9 | 0.0279 | RanBP1 (Ran-binding protein 1) |
| AF058718 | 2.9 | 0.0386 | putative 13 S Golgi transport complex |
| U84011 | 2.9 | 0.0279 | glycogen debranching enzyme isoform 6 (AGL) |
| D38524 | 2.8 | 0.0279 | 5′-nucleotidase |
| AF043325 | 2.8 | 0.0017 | N-myristoyltransferase 2 |
| X97335 | 2.7 | 0.0437 | kinase A anchor protein |
| M37721 | 2.7 | 0.0279 | peptidylglycine alpha-amidating monooxygenase |
| Y00757 | 2.7 | 0.0013 | polypeptide 7B2 |
| X95592 | 2.6 | 0.0130 | C1D protein |
| AF051321 | 2.6 | 0.0076 | Sam68-like phosphotyrosine protein alpha (SALP) |
| K03000 | 2.6 | 0.0013 | aldehyde dehydrogenase 1 |
| M96860 | 2.6 | 0.0418 | dipeptidyl aminopeptidase like protein |
| U35451 | 2.6 | 0.0013 | heterochromatin protein p25 |
| tigr:HG4074-HT4344 | 2.5 | 0.0057 | Rad2 |
| U03634 | 2.5 | 0.0418 | P47 LBC oncogene |
| U14518 | 2.5 | 0.0213 | centromere protein-A (CENP-A) |
| J04031 | 2.5 | 0.0133 | methylenetetrahydrofolate dehydrogenase- |
| | | | methenyltetrahydrofolate cyclohydrolase- |
| | | | formyltetrahydrofolate synthetase |
| X59543 | 2.4 | 0.0049 | M1 subunit of ribonucleotide reductase |
| AJ236876 | 2.4 | 0.0076 | poly(ADP-ribose) polymerase-2 |
| AF093774 | 2.4 | 0.0082 | type 2 iodothyronine deiodinase |
| U74324 | 2.4 | 0.0117 | guanine nucleotide exchange factor mss4 |
| U73737 | 2.4 | 0.0076 | hMSH6 |
| X06745 | 2.4 | 0.0025 | DNA polymerase alpha-subunit |
| AF060219 | 2.4 | 0.0130 | RCC1-like G exchanging factor RLG |
| AF005043 | 2.3 | 0.0049 | poly(ADP-ribose) glycohydrolase (hPARG) |
| D61391 | 2.3 | 0.0013 | phosphoribosypyrophosphate synthetase- |
| | | | associated protein 39 |
| AF068754 | 2.3 | 0.0049 | heat shock factor binding protein 1 HSBP1 |
| Y10746 | 2.3 | 0.0013 | MBD 1 |
| U31930 | 2.3 | 0.0013 | deoxyuridine nucleotidohydrolase |
| M97287 | 2.3 | 0.0025 | MAR/SAR DNA binding protein (SATB1) |
| U84720 | 2.2 | 0.0049 | mRNA export protein (RAE1) |
| AF000993 | 2.2 | 0.0117 | ubiquitous TPR motif, X isoform (UTX) |
| U04840 | 2.2 | 0.0117 | onconeural ventral antigen-1 (Nova-1) |
| D14041 | 2.2 | 0.0130 | H-2K binding factor-2 |
| D55654 | 2.2 | 0.0049 | cytosolic malate dehydrogenase |
| U36336 | 2.2 | 0.0279 | lysosome-associated membrane protein-2b |
| | | | (LAMP2) |
| L36140 | 2.1 | 0.0130 | DNA helicase (RECQL) |
| AF047442 | 2.1 | 0.0279 | vesicle trafficking protein sec22b |
| AF084481 | 2.1 | 0.0082 | transmembrane protein (WFS1) |
| U53209 | 2.1 | 0.0279 | transformer-2 alpha (htra-2 alpha) |
| L24521 | 2.1 | 0.0049 | transformation-related protein mRNA |
| L42572 | 2.1 | 0.0130 | p87/89 gene |
| L37043 | 2.1 | 0.0013 | casein kinase I epsilon |
| AJ001258 | 2.1 | 0.0279 | NIPSNAP1 protein |
| AJ005896 | 2.1 | 0.0437 | JM4 protein |
| U87459 | 2.1 | 0.0117 | autoimmunogenic cancer/testis antigen NY-ESO-1 |
| M30938 | 2.0 | 0.0130 | Ku (p70/p80) subunit |
| U59151 | 2.0 | 0.0279 | Cbf5p homolog (CBF5) |
| AB010882 | 2.0 | 0.0279 | hSNF2H |
| AJ132917 | 2.0 | 0.0130 | methyl-CpG-binding protein 2 |
| U96915 | 2.0 | 0.0049 | sin3 associated polypeptide p18 (SAP18) |

| | |
|---|---|
| EST's/Unknown | N = 153 |
| function | |

Clustering. The stringent data filtering for significant and consistent changes permitted identification of biologically relevant gene clustering in human endometrium during the window of implantation versus the late proliferative phase. We performed unsupervised cluster analysis, based on NCBI/Entrez/OMIM database search, which allowed grouping of genes into several categories (Table 2 and Table 3). The most markedly up-regulated genes (categories in descending order of maximal fold change) include those involved in cholesterol trafficking and transport (apolipoprotein E and D), prostaglandin biosynthesis and action (phospholipase A2 and the PGE2 receptor), proteoglycan synthesis (glucuronyltransferase I), and a variety of secretory proteins, including glycodelin (pregnancy-associated endometrial α₂ globulin), mammaglobin (a member of the uteroglobin family), members of the Wnt regulation pathway (Dickkopf-1), IGFBP family and TGF-βsuperfamily. Additional genes were upregulated, including G0S2 (a cell cycle switch protein), several genes involved in signal transduction, nitric oxide metabolism (arginase II), and extracellular matrix components/cell adhesion molecules, including osteopontin and laminin subunits. Also, of note are the marked up-regulation of genes for neuromodulator synthesis/receptors (GABA_{A} receptor π subunit), immune modulators [e.g., natural killer-associated transcript (NKAT) 2, members of the complement family, and interferon-induced genes (interferon γ-inducible indoleamine 2,3-dioxygenase (IDO)], genes involved in detoxification (several types of metallothioneins and glutathione peroxidase), phospholipid binding proteins (annexins), as well as some proteases, transcription factors and structural/cytoskeletal proteins. Among several gene families not heretofore known to exist in endometrium are members of water and ion transport that are common to the gastrointestinal epithelial mucosa and other mucosal surfaces [e.g., Clostridia Perfringens Enterotoxin (CPE)-1 receptor and the sulfonylurea receptor (K⁺ ion channel)]. Several genes for other cellular functions were also up-regulated.

The most abundantly down-regulated genes involved secretory proteins, including intestinal trefoil factor (a member of a family of proteins that maintains intestinal luminal epithelial cell integrity) and proteases, such as matrilysin (matrix metalloproteinase 7), dipeptidyl aminopeptidase and carboxypeptidase E. Also markedly down-regulated genes included those for G protein-coupled receptor signaling: G-protein coupled receptor kinase and G-protein gamma-11 subunit. Also, marked down regulation of calcineurin (a protein involved in Ca²⁺ signaling) was observed, as well as some members of the Wnt pathway [frizzed related protein (FrpHE) and secreted frizzed related protein (FRP)], genes for TGF-β signaling (Smad 1), the peroxisome proliferator activated receptor and members of the fibroblast growth factor receptor family. Select extracellular matrix/cell adhesion molecules were down regulated, including α-1 type XVI collagen and the extracellular matrix protein, tenascin-C. Numerous genes corresponding to transcription factors were found to be down-regulated during the implantation window, including homeobox genes (MSX-2, HOX-7), Kruppel family of zinc finger proteins, the erg protein (ets-related gene), several proto-oncogenes (c-fos, BMI-1 and others), apoptosis/inhibitors (TRAIL receptor 2), and immune modulators (MHC class II subunits). Of interest is the observed down-regulation of vasoactive substances (endothelin 3 and VEGF), several cell cycle regulators, and genes whose products have relevance to steroid hormone actions (putative progesterone binding protein/progesterone receptor membrane component 1 (PGRMC1) and steroid receptor coactivator 1e). Down regulation of several transporters and calcium channel subunits, structural and cytoskeletal proteins, and ion binding proteins were observed, as well as genes for other cellular functions.

Since clinical endometrial biopsy samples contain a mixture of different cell populations, including glandular and surface epithelial cells, stromal cells, and vascular, smooth muscle, and blood cell components, it is anticipated that many genes and gene families participating in different processes would be represented in the microarray data. In addition, previously documented genes in these cellular components would be anticipated to be detected in the GeneChip analysis. Reassuringly, many genes known to be significantly up-regulated in human endometrium during the secretory phase were up-regulated >2-fold and included (Table 2): pregnancy-associated endometrial α₂-globulin (glycodelin, an exclusively endometrial epithelial cell product predominantly expressed in the secretory phase of the cycle); IGFBP-2 which is exclusively an endometrial stromal cell product upregulated in the secretory phase; osteopontin, an endometrial epithelial-specific protein; prostaglandin E2 receptor; transforming growth factor (TGF) β-Type II receptor (R); and interleukin (IL)-15 and its receptor. Others, such as insulin-like growth factor-II (IGF-II), plasminogen activator inhibitor (PAI-I), urokinase receptor, tissue inhibitor of metalloproteinase-3 (TIMP-3), fibroblast growth factor (FGF)-6 and FGF-8, and IGFBP-1 were also up-regulated, although they did not reach statistical significance by non-parametric testing. For the genes down regulated in the window of implantation (Table 3), significant down-regulation of matrilysin (MMP-7) and tenascin-C was detected, consistent with previous studies demonstrating their decreased expression in secretory, compared to proliferative phase, endometrium.

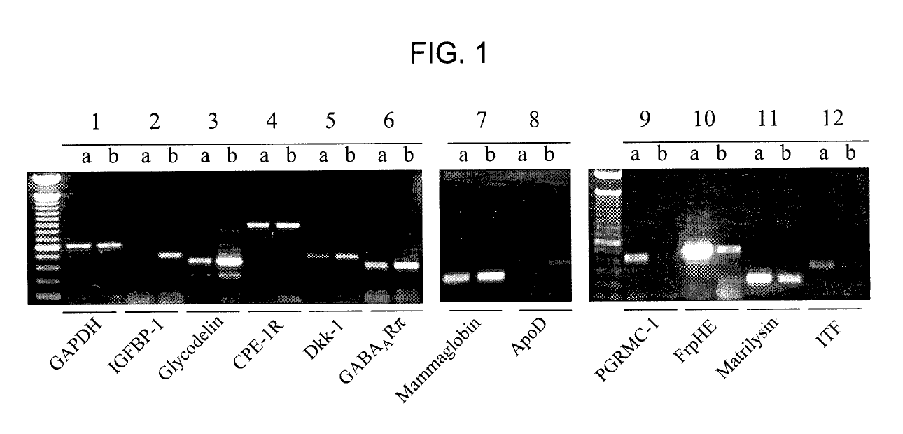

Validation of Gene Expression. We validated expression of select up-regulated and down-regulated genes using two approaches: RT-PCR and Northern analyses with RNAs from late proliferative and window of implantation endometrial biopsy tissue samples and RT-PCR using RNA isolated from cultured human endometrial glandular and stromal cells. The results are shown in FIGS. 1-3. For the RT-PCR studies, the primer sets are shown in Table 1. Although quantitative PCR was not performed, the RT-PCR data in FIG. 1 demonstrate clearly in the implantation window compared to late proliferative phase endometrium, upregulation of IGFBP-1, glycodelin, CPE-1 R, Dkk-1, GABA_{A}receptor π subunit, mammaglobin, and ApoD, and down-regulation of PGRMC1, frpHE, matrilysin, and ITF. These data are consistent with the observations from the microarray data (Tables 2 and 3).

FIG. 1. Validation of selected genes >2-fold up- or down-regulated during the window of implantation in human endometrium by RT-PCR. Endometrial biopsy samples from late proliferative phase (n=3) and the window of implantation (n=3) were processed for total RNA, and representative results are shown. RT-PCR was conducted with specific primer sets shown in Table 1, using the samples from the proliferative phase (lanes a) or window of implantation (lanes b). Appropriate sized products corresponding to glyceraldehyde 3-phosphate dehydrogenase (GAPDH) (lanes 1a, 1b), insulin-like growth factor binding protein-1 (IGFBP-1) (lanes 2a, 2b), glycodelin (lanes 3a, 3b), Clostridia Perfringens Enterotoxin-1 receptor (CPE-1 R) (lanes 4a, 4b), Dickkopf-1 (Dkk-1) (lanes 5a, 5b), gamma aminobutyric acid-A receptor π subunit (GABA_{A} R π) (lanes 6a, 6b), mammaglobin (lanes 7a, 7b), Apolipoprotein D (ApoD) (lanes 8a, 8b), Progesterone receptor membrane component 1/putative progesterone binding protein (PGRMC-1) (lanes 9a, 9b), Frizzled related protein (FrpHE) (lanes 10a, 10b), matrilysin (lanes 11a, 11b) and intestinal trefoil factor (ITF) (lanes 12a, 12b) are shown.

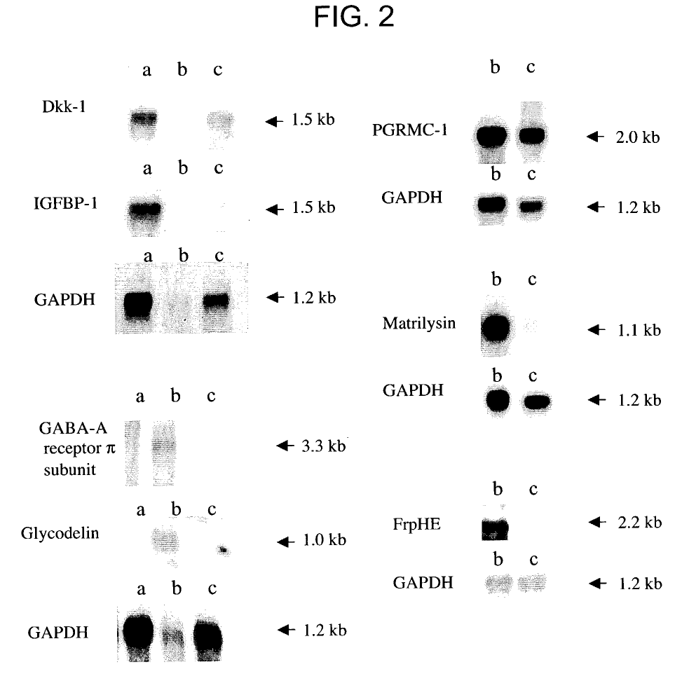

Northern analyses were conducted to validate further select changes in gene expression in the implantation window versus the late proliferative phase, and a representative set of Northern blots are demonstrated in FIG. 2. Densitometric analyses were conducted, and means values of relative mRNA expression were derived after normalization of signals to GAPDH. Fold-changes between the implantation window and the proliferative phase were then calculated. These data demonstrate up-regulation of Dikkopf-1 (Dkk-1) (3.2-fold), IGFBP-1 (2.8-fold), GABA_{A} receptor π subunit (24.6-fold), and glycodelin (3.1-fold), and the down-regulation of PGRMC-1 (1.3-fold), matrilysin (20.0-fold), and frizzled related protein (FrpHE, 50-fold). The data are consistent with and validate those obtained through the microarray expression profiling analysis, although the levels of fold-change are not the same as in the microarray analysis (Tables 2 and 3) and would not be expected to be the same.

FIG. 2. Northern analysis demonstrating up-regulation of Dkk-1, IGFBP-1, GABA_{A} R n subunit, glycodelin, and down-regulation of PGRMC-1, matrilysin and FrpHE in the secretory phase (implantation window, lane c), compared to the proliferative phase (lane b). Placental basal plate with decidua (lane a) is shown as a positive control for Dkk-1 and IGFBP-1 on the left panel. GAPDH hybridization of respective blots are shown for comparison.

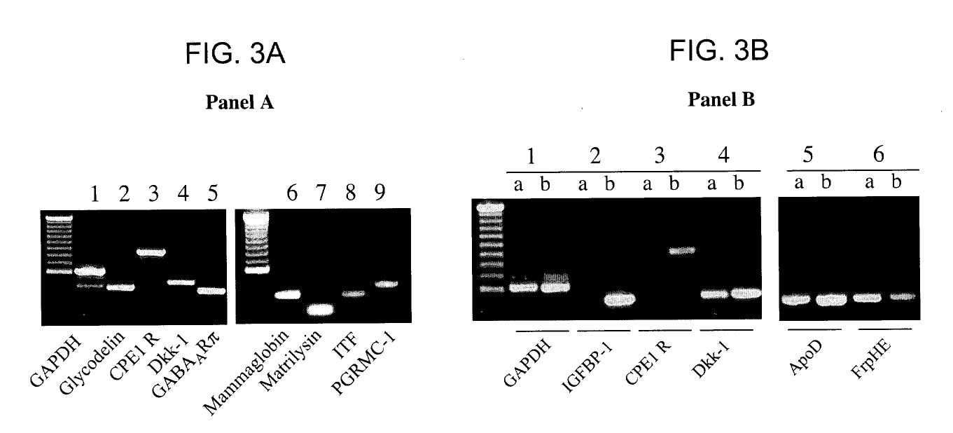

RT-PCR experiments using RNA from cultured endometrial epithelial and stromal cells and the primers listed in Table 1, revealed the following (FIG. 3): PCR products corresponding to glycodelin (positive control), the CPE-1 receptor, Dkk-1, the GABA_{A} receptor π subunit, mammaglobin, matrilysin, intestinal trefoil factor, and PGRMC-1 were all expressed in human endometrial epithelial cells (panel A), demonstrating their expression in this cell type in human endometrium. With cultured human endometrial stromal cells (FIG. 3, Panel B), up-regulation of the CPE-1 receptor, Dkk-1, ApoD, and IGFBP-1 (positive control) and down regulation of frizzled related protein (frpHE) upon in vitro decidualization with progesterone after e stradiol priming were observed. The GABA_{A} receptor π subunit, mammaglobin, matrilysin, intestinal trefoil factor, and PGRMC-1 were not detected in isolated and cultured endometrial stromal cells before or after decidualization.

FIGS. 3A-B. Expression of selected genes in cultured human endometrial epithelial (Panel A) and stromal (Panel B) cells by RT-PCR. Panel A Lane 1, GAPDH (control); lane 2, Glycodelin; lane 3, CPE-1 R; lane 4, Dkk-1; lane 5, GABA_{A} R π subunit; lane 6, Mammaglobin; lane 7, Matrilysin; lane 8, ITF; lane 9, PGRMC-1. Panel B demonstrates RT-PCR products using endometrial stromal cells non-decidualized (lanes “a”) or decidualized (lanes “b”) with progesterone after estradiol priming, as described in Material and Methods. Lanes 1a, 1b: GAPDH (control); lanes 2a, 2b, IGFBP-1; lanes 3a, 3b, CPE-1 R; lanes 4a, 4b, Dkk-1; lanes 5a, 5b, Apolipoprotein D; lanes 6a, 6b, FrpHE. Experiments were conducted with isolated cells from 5 different samples. Representative results are shown.

Molecular mechanisms that involve apposition, attachment, and intrusion of an implanting embryo into human endometrium are beginning to be appreciated. Most of what is believed to occur during human implantation is derived from animal models that have been invaluable, especially when the reproductive phenotype involves implantation failure. A limiting factor in research with human endometrium has been the availability of appropriately characterized clinical specimens. Herein, we have presented global gene profiling of well characterized human endometrial biopsy samples that were obtained during the window of implantation, defined by timing to the LH surge and histologically confirmed. About one-third of the samples we collected had to be discarded because their histology was not consistent with normal temporal development in the cycles in which they were obtained. This observation underscores the need for precise histologic confirmation of endometrial samples prior to analysis. The approach taken in this study also demonstrates that reproducible and sensitive experimental methodology for global gene analysis can be applied to small quantities of human endometrial tissue. Similar approaches have been successfully pursued with whole tissues. The current study used high-density oligonucleotide microarray expression profiling which allowed profiling and interrogation of expression of 12,686 full-length genes and ESTs in human endometrial biopsies. The microarray technologies and the data presented herein highly support the use of this powerful approach to investigate molecular candidates involved in human uterine receptivity. Results from the human genome project suggest that we have interrogated about one-third to one-half of existing human genes, and thus, investigation of additional genes, as well as their validation, present a formidable task and await further investigation.

Endometrial biopsy specimens contain several cell populations and may differ in their complement of such populations. This heterogeneity may contribute to differences observed in relative expression of select genes between the implantation window and the late proliferative phase as assessed by the microarray approach versus Northern analysis or RT-PCR approaches. In addition, since different samples were used for the microarrays and the validation studies, subject-to-subject biologic variation in samples obtained in the same phase of would be anticipated. Also, in the microarray analysis, the mean of an individual gene readout from the samples in the window of implantation was compared to the mean of the same gene readout of the proliferative phase samples; whereas, in calculating the fold-change for a given gene analyzed by Northern analysis, the mean of the densitometric OD readings were calculated after normalization to GAPDH. We speculate that differences in the fold-change values between the two methodologies may also be due to the lower abundance of specific mRNAs in relation to the highly abundant GAPDH mRNA, especially since the microarray profile represents true abundance of each mRNA species globally within the tissue whereas Northern analysis reflects mRNAs of higher abundance and is poor in detecting very low abundance transcripts.

While this heterogeneity among samples may influence the relative expression of some genes, we confirmed previously documented genes within the window of implantation, such as the endometrial glandular-specific glycodelin and the stromal cell-specific, IGFBP-1 and IGFBP-2. Other molecules, such as the PGE2 receptor, interleukin-15, and the TGF-β type II receptor have all been reported to be up-regulated in human endometrium during the secretory phase in various cell types, further validating the approach taken herein. Down-regulation of matrix metalloproteinase-7 (matrilysin) has been demonstrated previously, as has the down-regulation of tenascin-C, a multifunctional extracellular matrix glycoprotein that is regulated by multiple soluble factors, integrins, and mechanical forces, and known to be highly expressed in the proliferative phase of the menstrual cycle compared to the secretory phase. While the data presented contribute to the molecular signature of the endometrium that defines the state of receptivity to embryonic implantation, localization of cell type expression for select genes is clearly needed and is underway in our laboratories. Also, while the validation studies presented herein support cell-specific expression for a few, selected genes and validate in a limited fashion the microarray data, they do not represent the full spectrum of cell types in the endometrium, underscoring the need for in situ hybridization studies and subsequent protein demonstration. In addition, endometrial proteins that require posttranslational modifications for their activity are not revealed by gene profiling techniques and require alternative methods of investigation.

The choice to compare gene expression profiles in the window of implantation to the late proliferative phase was made to focus on the comparison of genes expressed during peak exposure of the endometrium to estradiol and progesterone (window of implantation) versus peak estradiol (late proliferative phase). While many of the genes are known to be regulated by progesterone directly, e.g., glycodelin, IGFBP-1 and TIMP-3, regulation of others during the window of implantation likely derive from progesterone-induced (or suppressed) paracrine products that are mediators of the progesterone response. The finding of unique gene families, not previously known to be expressed in human endometrium or to be regulated by progesterone provides new avenues of investigation which is an advantage of this unbiased technique and which transcends the goals of the current investigation to other fields. In addition, why more genes are down-regulated than up-regulated is an interesting observation and we speculate that since during the implantation window, compared to the late proliferative phase, estrogen receptors are down-regulated in endometrial epithelial cells, genes that were up-regulated by estradiol in the proliferative phase are now down-regulated due to the loss of estradiol action. In addition, direct down-regulation by progesterone and multiple progestomedins during the implantation window resulting in more down-regulated genes compared to upregulated genes.

Up-regulated Genes. Several genes and gene families that are up-regulated in the window of implantation warrant further discussion. Apolipoprotein E is the most abundantly (100-fold) up-regulated gene in the window of implantation. It binds hydrophobic molecules and is important in cholesterol transport and trafficking. Local production of apo E in steroidogenic tissues, particularly the ovary, has been reported, through mechanisms involving the LDL receptor family. The high expression of apo E (and apo D) in the endometrium suggests an important role for it in cholesterol transport in this tissue, perhaps for steroid hormone biosynthesis or steroid hormone binding.

Phospholipase A2 (PLA2), the second most abundantly (18-fold) up-regulated gene in the window of implantation, belongs to a family of enzymes (secreted, membrane bound, Ca⁺⁺-dependent) that catalyze the hydrolysis of membrane glycerophopholipids, resulting in the release and metabolism of arachadonic acid and generation of lipid signals: platelet-activating factor, lysophosphatidic acid, prostaglandins (PG) and leukotrienes. The importance of PG action during the window of implantation is underscored by the concomitant (4-fold) up-regulation of the PGE2 receptor (Table 2). PLA2 is also involved in calcium influx into non-excitable cells and in the modulation of TNF-α and IL-1β-induced NF-kappa B activation, which is important in endometrial function. PGs are important for vascular permeability and endometrial decidualization. Further definition of mechanisms underlying PLA2 and PG actions during the implantation window are major challenges for further investigation.

Of interest in the implantation window is the finding of expression and marked (12-fold) up-regulation of mammaglobin, classically known as a breast-specific uteroglobin family member. Mammaglobin B has been identified in rat uterus, and uteroglobin has been well characterized in rabbit and human endometrium and is known to be regulated by progesterone. Several properties of members of the uteroglobin family have been identified, including serving as a substrate for transglutaminases and acting as an anti-inflammatory agent by inhibiting phospholipase A2. It is striking that both PLA2 and mammaglobin, a putative inhibitor of this enzyme, are so markedly up-regulated during the window of implantation in human endometrium. Of course, mammaglobin, a member of the secretory lipophilin family of proteins that are prominent in glandular secretions and hormone-responsive tissues, may have other functions, yet to be identified in the implantation window in human endometrium.

Another inhibitor of PLA2 is annexin IV, a member of the annexins or lipocortin family of calcium-dependent phospholipid-binding proteins. Annexin IV, also known as placental anticoagulant protein II, has anticoagulant activity, as well. The upregulation of annexin IV, annexin II, and lipocortin-2 in the implantation window underscores the importance of regulating PLA2 activity and maintaining an environment for anti-coagulation during implantation.

Pregnancy-associated endometrial α₂ globulin, also known as glycodelin, is an endometrial epithelial-specific protein and is upregulated in human endometrium during the peri-implantation period and in the late secretory phase. Data in this study support these well established observations. Glycodelin belongs to a family of lipocalins that participate in regulation of the immune response that also includes α₁ microglobulin and the γ chain of complement factor 8. The lipocalins typically bind small hydrophobic molecules, like retinol and retinoic acid, although glycodelin does not bind these molecules.

The finding of members of the Wnt family is surprising. Of particular interest is the marked up-regulation of Dickkopf-1 (an inhibitor of Wnt signaling) and of LRP [low density lipoprotein (LDL) receptor like protein] and the down regulation of frizzled related protein (FrpHE), also an inhibitor of Wnt signaling. Dickkopf-1 inhibits Wnt signaling by binding LRP5/6, and FrHPE inhibits Wnt action by competitive binding to Wnt ligand(s). Wnt 7A −/− null mice are infertile and have complete absence of uterine glands and a reduction in mesenchymally-derived uterine stroma. We have localized Wnt 7A exclusively to epithelium and frizzled receptor to epithelium and stroma in human endometrium. It is possible that the Wnt family may play a role in epithelial-embryo and/or epithelial-stromal interactions and thus in uterine receptivity. The role of the Wnt family in human endometrium and implantation is currently under investigation in our laboratories.

Proteoglycans, extracellular matrix (ECM) proteins, and cell surface glycoproteins function in epithelial-embryonic interactions. T he ECM is also a reserve of many peptide growth factors and angiogenesis modulators, underscoring the importance of its regulation in events occurring in the endometrium. Of particular interest is the marked (16-fold) up-regulation of glucyronyltransferase I, a central enzyme in heparan/chondroitin sulfate and other proteoglycan biosynthesis. Also, significantly up-regulated (8-fold) during the window of implantation is the ECM protein, osteopontin, known to be progesterone-regulated and up-regulated in the mid-secretory phase in human endometrium. Osteopontin has been postulated to bridge embryo-epithelial attachment. Also, we found up-regulation of laminin B, and proline-rich protein, an ECM protein commonly found in intestinal epithelium.

A number of genes involved in immune modulation deserve special mention, although their cellular expression h as not yet been determined. These include (also see Table 2): natural killer-associated transcript 2 (NKAT-2), members of the complement family (including adipsin which is the same as complement D, decay-accelerating factor, and complement 1r), interleukin 15 and its receptor, NKG5 (an NK and T-cell specific gene strongly up-regulated upon cell activation), interferon γ-inducible indoleamine 2,3-dioxygenase (IDO), interferon regulatory factor 5, and lymphotaxin/SCM1γ (expressed in NK cells). Some of these immune modulators are well characterized in human endometrium and have functions related to NK cell differentiation (e.g., IL-15) and complement action, and may play key roles in immune tolerance of an implanting embryo (e.g., IDO may have a role in the prevention of allogeneic fetal rejection by tryptophan catabolism). The impressive regulation of immune modulators underscores the need for further investigation into this important group of gene families in the implantation process, especially in view of the controversies currently surrounding immune-based therapies for some infertility patients.

Of interest are transport proteins for water and ions that are common to kidney and gastrointestinal epithelium (Table 2). It is reasonable that mechanisms are conserved for water and ion transport—whether they be in the gut, the kidney or the endometrium. Finding expression of these transporters and their marked upregulation during the implantation window likely reflects the importance of water (and ion) shifts that take place across the epithelium and the importance of endometrial stromal edema that occur during the window of implantation. The gene for the Clostridia Perfringens Enterotoxin (CPE) 1 receptor, e.g. was upregulated 4-fold in the implantation window. This receptor is a tight junction protein component that forms pores for water transport in the gut, and in response to Clostridia Perfringens Enterotoxin results in massive water shifts into the intestinal lumen. It has been found to be abundantly expressed in the gastrointestinal tract and in the uterus. Whether this receptor is involved in water transport that occurs during the mid-secretory phase, is unknown. Further, its endogenous ligand in the endometrium (and its true function) await definition. The finding of the CPE-1 receptor and of a membrane protein potassium channel—the sulfonyl urea receptor, open new avenues of investigation in endometrial biology, focusing on, e.g., signaling from an embryo involving ion fluxes, with appropriate channels in place for such interactions.

Genes for members of the metallothionein family of proteins that are involved in detoxification and zinc binding are upregulated 2.3-5.8-fold during the implantation window. In zinc-deficiency and in metallothionein knockout mice there is an alteration of Th1 and Th2 cytokines. Since the ratio of Th1 to Th2 is believed to be important for successful implantation in humans, this gene family may provide a mechanism to regulate the immune balance for embryonic tolerance during implantation. Also of note is the up-regulation of genes governing intracellular Ca²⁺ signaling and Ca²⁺ homeostasis [annexin II], underscoring the importance of Ca²⁺ in the implantation window (5,6). Genes whose products are involved in G protein-coupled receptor desensitization, e.g., β-arrestin, β-adaptin, and clathrin, are up-regulated, supporting attenuated G-protein coupled receptor signaling in the implantation window. Cyclophilins are upregulated during the implantation window, and since they bind with Hsp 90 to inactivate steroid hormone receptors, they may contribute to the observed down-regulation of the estrogen receptor in endometrial epithelium between cycle days 20-24.

Up-regulation of the GABA_{A} receptor π subunit and documentation of its epithelial origin in human endometrium during the implantation window (Table 2 and FIGS. 1, 2 and 3) raise the issue of the role of neurotransmitters and of progesterone metabolism in this tissue. The GABA_{A} receptor has been reported in rat uterus and is important in the binding of reduced metabolites of progesterone in this tissue. Whether this is important in human endometrium remains to be determined. The observations of up-regulation of monoamine oxidase (important in norepinephrine synthesis) and diamine oxidase (DAO), well recognized in human endometrium, underscore the need to reach beyond conventional thinking about mechanisms operating in endometrial development and perhaps embryo-endometrial interactions. Cellular localization of these genes and their ligands (e.g., for the GABAA receptor π subunit) clearly need further definition. However, these findings and our recent findings of neuromodulators and their receptors in decidualized human endometrial stromal cells underscore further consideration of neurotransmitter receptors participating in signals from an implanting embryo during nidation into the endometrium. The roles of some of these receptors may have other functions, as has been shown for dopamine and morphine stimulating nitric oxide production by human endometrial glandular epithelial cells in culture.

Down-regulated Genes. Intestinal trefoil factor (ITF), a member of a family of secreted proteins that are expressed in the epithelial mucosal layer of the small intestine and colon, is the most markedly (50-fold) down regulated gene in human endometrium during the window of implantation (Table 3). Studies with ITF null (−/−) mice support a central role for ITF in maintenance and repair of the intestinal mucosa. Whether an analogous role is present in endometrium warrants further investigation.

Other markedly down regulated genes include some that are involved in G protein-coupled receptor signaling: G-protein-coupled receptor kinase (23-fold reduction); HM145 (a G-protein-coupled receptor for leukocyte chemoattractants, 11-fold reduction), and the G-protein gamma 11 subunit (4.7-fold reduction). Down-regulation of this signaling pathway raises questions of identifying ligand/receptor complexes using this pathway and why their down-regulation is important during the implantation window. This is notable, especially since this apparently is coordinated with up-regulation of G-protein receptor inhibitory factors.

Several peptidases were also found to be down-regulated during the implantation window (Table 3), including, matrilysin (24-fold), dipeptidyl amino peptidase (10-fold), carboxypeptidase E (9.7-fold), and cathepsin F (3-fold), suggesting that proteolysis is minimized during this part of the menstrual cycle. As has been shown for MMPs, inhibition of MMPs may be critical to the maintenance of endometrial tissue architecture, very important during the implantation window. Dipeptidyl amino peptidase is a brush-border membrane-bound enzyme in the kidney proximal tubule and has been implicated in regulation of the biologic activity of multiple hormones and chemokines. Carboxypeptidase E is a regulated secretory pathway (RSP) sorting receptor which regulates hormone, neuropeptide, and granin secretion in a calcium-dependent manner, important in prohormone processing, including pro-insulin and neurotransmitters. Down-regulation of these enzymes may be part of a local control mechanism for regulating peptide activity within the endometrium.

Several other genes were also markedly down-regulated, including MSX-2 (a homeobox gene, 9-fold), genes involved in calcium and ion transport, and calcineurin, a protein involved in Ca²⁺ signaling (7.5-fold). Calcineurin is important in the activation of T cells. Antigen recognition by T-cell receptors initiates signal transduction resulting in activation of tyrosine kinases, followed by phospholipase C (PLC) phosphorylation. This causes phosphatidyl inositol phosphate (PIP) phosphorylation to PIP3, elevating intracellular Ca²⁺ and 1,2-diacylglycerol. Through the increased level of free Ca²⁺, a complex of calmodulin and calcineurin is formed. Calcineurin is a Ca-/calmodulin-dependent ser-thr phosphatase and dephosphorylates the nuclear factor of activating T cells (NF-AT). In the dephosphorylated form, NF-AT crosses into to the nucleus to function as a transcription activator for IL-2 expression. Down regulation of calcineurin in endometrium would suggest limitation of NF-AT activation in this tissue. In addition, several transcription factors are down-regulated. Of note is the erg protein, a member of the ets family, important in regulation of extracellular matrix. With the dynamic changes in the extracellular matrix that occur in endometrium during the window of implantation and during early pregnancy, ets family members may play an important role.

Semaphorin E and semaphorin III family homologue were found to be downregulated (6- and 3-fold, respectively) during the implantation window. Semaphorin III interacting with its receptor can result in either chemorepulsion or chemoattraction of developing axons, depending on levels of cellular cyclic GMP. Finding the semaphorins and neurotransmitter receptors, as described above, suggests that perhaps we should be looking at other systems, such as ion signaling and chemoattractants/repellants for mechanisms to guide an embryo within the endometrium, analogous to neurotransmitter and semaphorin action in the neuronal system.

Of note also is down-regulation during the implantation window of the vasoactive factor, endothelin 3, and the angiogenic factor, VEGF (Table 3). Minimizing vasoconstriction is teleologically sound during a period that requires enhanced blood flow to the conceptus. Why VEGF is down regulated is not clear, and conflicting reports have been reported on cyclic variations of this angiogenic factor in human endometrium. However, Semaphorin III and VEGF compete for the same receptor, neuropilin-1 and this interaction results in inhibition of aortic endothelial cell migration. Interactions between the angiogenic system and the neuronal guidance system suggest potential new mechanisms for regulation of cellular motility in the endometrium during the implantation window, if indeed this extrapolation can be made.

The current study opens new conceptual approaches to mechanisms involved in the steroid hormone-dependent differentiation of the endometrium in the secretory phase of the menstrual cycle and mechanisms underlying endometrial development optimal for embryonic implantation and for embryo-endometrial interactions. The classes of molecules described herein support the following model. As an embryo attaches to the endometrial epithelium, bridging to cell surface carbohydrates and proteins is important, and mechanisms must be in place in the maternal endometrium for synthesis of these molecules. Once attachment occurs, a set of mechanisms is put into motion for endometrial-stromal interactions, intrusion of the trophoblast into the stromal compartment, and guidance of the trophoblast to the maternal spiral arteries, while maintaining integrity of the ECM and anticoagulation. It is envisioned that embryo-endometrial interactions involve ion transport and signaling through paracrine mechanisms via growth factors and cytokine families, as well as adaptation of guidance mechanisms similar to those used in angiogenesis and neuronal migration to target the trophoblast through the stroma to reach to the maternal vasculature. The immune system must facilitate tolerance of the implanting allograph and other protective mechanisms (anti-bacterial, detoxification, e.g.,) are likely to be important to maximize viability of the implanting conceptus. This model provides a framework for the role of the genes identified in this study in these processes for further investigation. It is important to note that despite the anticipated interactions between the endometrium and the conceptus, based on gene expression in the endometrium during the implantation window described herein, the microarray approach provides a static snapshot of gene expression and does not reveal the dynamic dialog that occurs minute-to-minute during embryonic implantation. Nonetheless, it does provide insight into the molecular pathways, molecular signals, and physiologic processing that await an embryo should nidation occur.

Validation of functions for genes in the window of implantation will derive, in the future, from animal models of homologous recombination and gene knockouts, transgenic mice, studies in nonhuman primates and other species whose endometrium and implantation processes are similar to those in humans, and further studies in human endometrial disorders related to implantation-based infertility. We believe that the current study provides the basis for defining markers of uterine receptivity during the window of implantation in human endometrium. Recent applications of global gene profiling relevant to implantation include a study by Reece et al in which uterine genes and gene families were characterized in mice during implantation in a variety of pregnancy models, and by Aronow et al on genes involved in human trophoblast differentiation. Information from these studies and the current study in human endometrium should further advance our knowledge about implantation in humans.

### Example 2

### Genes Differentially Expressed in Endometriosis

Materials and Methods

Tissue Specimen

Tissues

Endometrial biopsies were obtained from normally cycling women after informed consent, under a protocol approved by the Stanford University Committee on the Use of Human Subjects in Medical Research and the Human Subjects Committees at the University of North Carolina, Vanderbilt University, and the University of California at San Francisco. All specimens were obtained in accordance with the Declaration of Helsinki. A total of 20 biopsy samples were obtained during the window of implantation (mid-secretory phase/peak estradiol and progesterone) which were timed to the LH surge (LH+8 to LH+10, where LH=0 is the day of the LH surge) from women without (N=12) and with (N=8) mild/moderate endometriosis. Timing to the LH surge assured sampling during the window of implantation. Of the 20 biopsies, 15 were used for microarray studies and 5 were used for Northern or Dot Blot analyses and RT-PCR validation (vide infra) and 2 were used for both. The subjects were between 28-39 years old, had regular menstrual cycles (26-35 days), were documented not to be pregnant, and were taking no medications. Endometrial biopsies were performed with Pipelle catheters under sterile conditions, from the uterine fundus. A portion of each sample was processed for histologic confirmation, and the remainder was immediately frozen in liquid nitrogen for subsequent RNA isolation. Secretory phase histologies were confirmed independently by three observers: LCG, BAL, and an independent pathologist.

Gene Expression Profiling

RNA Preparation/Target Preparation/Array Hybridization and Scanning

Of the fifteen window of implantation samples used for microarray analysis, N=8 were from patients with surgically confirmed pelvic endometriosis (mild/moderate disease) and N=7 from women without endometriosis. The latter samples served as the basis for our recent study comparing gene expression in the window of implantation compared to the late proliferative phase in normally cycling women without endometriosis. Each endometrial biopsy sample was processed individually for microarray hybridization following the Affymetrix (Affymetrix, Santa Clara, Calif.) protocol. Briefly, poly (A)⁺-RNA was initially isolated from the tissue samples using Oligotex® Direct mRNA isolation kits (Qiagen, Valencia, Calif.), and a T7-(dT)₂₄ oligo-primer was subsequently used for double stranded cDNA synthesis by the Superscript Choice System (InVitrogen, Carlsbad, Calif.). In vitro transcription was subsequently carried out with Enzo BioArray High Yield RNA T7 Transcript Labeling Kits (ENZO, Farmingdale, N.Y.) to generate biotinylated cRNAs. After chemical fragmentation with 5× fragmentation buffer (200 mM Tris, pH 8.1, 500 mM KOAc, 150 mM MgOAc), biotinylated cRNAs were mixed with controls and were hybridized to Affymetrix Genechip Hu95A oligonucleotide microarrays on an Affymetrix fluidics station at the Stanford University School of Medicine Protein and Nucleic Acid (PAN) Facility. Fluorescent labeling and laser confocal scanning were conducted in the PAN Facility and generated the data for analysis.

Data Analysis

The data were analyzed using GeneChip® Analysis Suite v4.01 (Affymetrix), GeneSpring v4.0.4 (Silicon Genetics, Redwood City, Calif.), and Microsoft Excel/Mac2001 software, as described. Kao et al. (2002) Endocrinol. 143:2119-2138. To assess the expression ratios between the two groups, expression profile data were first prepared using GeneChip Microarray Analysis Suite® and subsequently exported to GeneSpring for rank-sum normalization and statistical analysis. Chip-to-chip variability is low; e.g., when RNA from one endometrial tissue sample was subjected to two independent hybridizations, less than 2.7% of the total genes on the array showed more than 3-fold variation, providing a greater than 95% confidence level, consistent with the manufacturer's published claims. Lipshutz et al. (1999) Nat Gene 21:20-24; and Wodicka et al. (1997) Nat. Biotech. 15:1359-1367. With GeneSpring v4.0.4 software, within each hybridization panel the 50th percentile of all measurements was used as a positive control for normalization, and each measurement for each gene was divided by this control, utilizing the bottom tenth percentile for background subtraction. Between different hybridization outputs/arrays, each gene was further normalized by synthesizing a positive control for that gene, using the median of the gene's expression values over all samples of an experimental group, and dividing the measurements for that gene by this positive control, as per the manufacturer's instructions. Mean values were then calculated among individual experimental groups for each gene probe-set, and between-group “fold-change” ratios were derived [i.e., with endometriosis (N=8): without disease (N=7) ratios]. A difference of 2-fold was applied to select up-regulated and down-regulated genes. Non-parametric Mann-Whitney U test was conducted to calculate the p-values, applying p<0.05 to assign statistical significance between the two groups.

Validation of Gene Expression Data

Reverse Transcription-Polymerase Chain Reaction (RT-PCR)

Genes of different expression fold changes were randomly selected for validation by RT-PCR and/or Northern or dot blot analyses. Total RNA from whole endometrial tissue was isolated using Trizol (Invitrogen) protocol, digested with DNase (Qiagen) and then purified by RNeasy Spin Columns (Qiagen). Four window of implantation endometrial biopsy samples were used for these experiments: two from female infertility patients with surgically proven endometriosis and two from normal fertile volunteers. Reverse transcription was first performed with Omniscript kit (Qiagen) for 1 h at 37° C., followed by a 50 μl reaction volume PCR with 40 pmol of specific oligo-primer pairs (Table 4) and Taq polymerase (Qiagen), using the Eppendorf Mastercycler Gradient. The amplification consisted of a hot start at 94° C. for 15 min, followed by 25-35 cycles of: denaturation at 94° C., annealing at optimized temperature, and extension at 72° C., each for 45 sec. Specific oligo-primer pairs were derived from public databases and synthesized by the PAN Facility, Stanford University School of Medicine. All PCR products used for Northern and dot blot analyses were purified with QIAquick Gel Extraction Kit (Qiagen) and verified by the Stanford PAN Sequencing Facility.

TABLE 4

PCR primer pairs sequences
and anticipated product length

| | | |
|---|---|---|
| Primer | Sequence | Length |

| | | |
|---|---|---|
| GAPDH | (S) 5′ CACAGTCCATGCCATCACTGC 3′ | 609 bp |
| | (SEQ ID NO:23) | |
| | (AS) 5′ GGTCTACATGGCAACTGTGAG 3′ | |
| | (SEQ ID NO:24) | |
| Semaphorin | (S) 5′ CTGATGGGAGATACCATGTC 3′ | 380 bp |
| E | (SEQ ID NO:25) | |
| | (AS) 5′ TCCTCTGCATTGAGTCAGTG 3′ | |
| | (SEQ ID NO:26) | |
| Collagen α2 | (S) 5′ TGCACCACTTGTGGCTTTTG 3′ | 425 bp |
| type I | (SEQ ID NO:27) | |
| | (AS) 5′ AAGCTTCTGTGGAACCATGG 3′ | |
| | (SEQ ID NO:28) | |
| ANK-3 | (S) 5′ AATGCTTGCCGCTTTAGAGG 3′ | 353 bp |
| | (SEQ ID NO:29) | |
| | (AS) 5′ ATCGACTAGGTCATCCAGTG 3′ | |
| | (SEQ ID NQ:30) | |
| BSEP | (S) 5′ AATGTCAAGTGGCAGCTCAG 3′ | 326 bp |
| | (SEQ ID NO:31) | |
| | (AS) 5′ CCCTATCCTTAGCCTTAGAG 3′ | |
| | (SEQ ID NO:32) | |
| Integrin α2 | (S) 5′ CTCTTCGGATGGGAATGTTC 3′ | 602 bp |
| | (SEQ ID NO:33) | |
| | (AS) 5′ TTGCAACCAGAGCTAACAGC 3′ | |
| | (SEQ ID NO:34) | |
| PD-EGF | (S) 5′ ATGGATCTGGAGGAGACCTC 3′ | 390 bp |
| | (SEQ ID NO:35) | |
| | (AS) 5′ AGAATGGAGGCTGTGATGAG 3′ | |
| | (SEQ ID NO:36) | |
| GlcNAc | (S) 5′ TGATTCCCTGTGGTGATACC 3′ | 296 bp |
| | (SEQ ID NO:37) | |
| | (AS) 5′ CCCACTTCAAAATGGAAGGC 3′ | |
| | (SEQ ID NO:38) | |
| Ephrin | (S) 5′ AAACAAGCTGTGCAGGCATG 3′ | 349 bp |
| | (SEQ ID NO:39) | |
| | (AS) 5′ CCTTACAGCTACACTCTAAG 3′ | |
| | (SEQ ID NO:40) | |
| Glycodelin | (S) | 420 bp |
| | 5′ AAGTTGGCAGGGACCTGGCACTC 3′ | |
| | (SEQ ID NO:41) | |
| | (AS) | |
| | 5′ ACGGCACGGCTCTTCCATCTGTT 3′ | |
| | (SEQ ID NO:42) | |

GAPDH = glyceraldehyde 3-phosphate dehydrogenase
ANK-3 = ankyrin G
BSEP = bile salt export pump
PD-EGF = platelet-derived endothelial cell grwoth factor
GlcNAc = N-actelyglucosamine-6-O-sulfotransferase

Northern and Dot Blot Analyses

Five window of implantation endometrial biopsy samples were used for these experiments, 2 from patients with endometriosis previously used in RT-PCR validation, and 3 from normal fertile volunteers, not used before. Total RNA (10-20 μg) was denatured and electrophoresed on 1% formaldehyde agarose gels and transferred for Northern analyses, or directly blotted for dot blot analysis through the Convertible Filtration Manifold System (Invitrogen), onto Nylon membranes. Specific radioactive probes were generated with Ready-to-Go random primer kit (Pharmacia Biotech, Peapack, N.J.), using PCR generated cDNAs, ranging 296-609 bp, and ³²αP-dCTP (NEN Life Science Products, Boston, Mass.), followed by MicroSpin S-200 HR Columns (Pharmacia) cleanup. Membranes were prehybridized at 68° C. for 60 min in ExpressHyb buffer (Clontech, Palo Alto, Calif.) containing salmon sperm DNA (Invitrogen), and hybridization carried out for another hour at 68° C. using buffer containing 1-2×10⁶ cpm/ml of labeled probe. After washing according to the manufacturer's instructions, membranes were exposed to Kodak MS X-ray films, scanned by Bio-Rad GS-710 Imaging Densitometer (Bio-Rad, Hercules, Calif.), and analyzed by its accompanied software Quantity One, v.4.0.2. GAPDH mRNA intensities were used for normalization prior to comparison. Mean values of relative expression intensities from different blots were used for final data presentation. Stripping and reprobing were performed using the same membranes.

Results

Data Analysis

The data were analyzed using GeneChip® Analysis Suite v4.01, GeneSpring v4.0.4, and Microsoft Excel/Mac2001, as detailed in Materials and Methods. As generally adopted for oligonucleotide microarray profile analysis, a minimal change of 2-fold was applied to select up-regulated and down-regulated genes. Nonparametric statistical testing was subsequently applied with a p-value of 0.05 used to designate significance between groups. Applying this strategy, we identified in the window of implantation in endometrium from women with versus without endometriosis, 91 genes that were significantly upregulated, of which 28 were ESTs, and 115 genes that were significantly down-regulated, of which 29 were ESTs. Table 5 and Table 6 show, in descending order, respectively, the fold-increase and fold-decrease, the p-values (p<0.05), and the GenBank accession numbers for the 63 specifically up-regulated genes and the 86 down-regulated genes in eutopic endometrium of women with endometriosis during the window of implantation, compared to normal fertile women, according to clustering assignments (vide infra).

TABLE 5
Genes Up-Regulated In Women With Endometriosis

| | GeneBank | | | |
|---|---|---|---|---|
| Function/Grouping | ID | Fold Up | p-value | Description (N = 91) |
| apoptosis related | U28015 | 100.0 | 0.0469 | cysteine protease (ICErel-III) |
| posttranslational protein | U47054 | 100.0 | 0.0156 | putative mono-ADP-ribosyltransferase |
| modification | | | | (htMART) |
| RNA processing | U63289 | 100.0 | 0.0080 | RNA-binding protein CUG-BP/hNab50 |
| | | | | (NAB50) |
| transporter | AF091582 | 100.0 | 0.0365 | bile salt export pump (BSEP) |
| voltage-dependent anion | AJ002428 | 100.0 | 0.0156 | VDAC1 pseudogene |
| channel/outer membrane | | | | |
| pore-forming protein | | | | |
| zinc finger protein | AF104902 | 100.0 | 0.0280 | ZIC2 protein (ZIC2) |
| zinc metalloenzymes | M33987 | 100.0 | 0.0015 | carbonic anhydrase I (CAI) |
| DNA mismatch repair | D38501 | 100.0 | 0.0080 | PMS7 mRNA (yeast mismatch repair gene |
| | | | | PMS1 homologue) |
| DNA replication | X74331 | 100.0 | 0.0113 | DNA primase (subunit p58) |
| immune function/cytokine | V00540 | 100.0 | 0.0211 | leukocyte (alpha) interferon |
| immune/cytokine | M60556 | 100.0 | 0.0156 | transforming growth factor beta-3 |
| immune function/cytokine— | L42243 | 27.1 | 0.0113 | interferon receptor (IFNAR2) gene |
| receptor | | | | |
| immune function | J03507 | 6.3 | 0.0469 | complement protein component C7 mRNA, |
| immune function/cytokine— | S71043 | 2.6 | 0.0037 | immunoglobulin A heavy chain allotype 2 |
| receptor | | | | |
| secretory protein | M25756 | 100.0 | 0.0113 | secretogranin II gene |
| secretory protein | X07704 | 23.9 | 0.0280 | PRB4 gene, allele M |
| secretory protein | AB000220 | 4.6 | 0.0113 | semaphorin E |
| secretory protein | AB000220 | 3.6 | 0.0080 | semaphorin E |
| tumor suppressor gene | AF010238 | 100.0 | 0.0056 | von Hippel-Lindau tumor suppressor (VHL) |
| | | | | gene |
| tumor suppressor gene | D50550 | 14.6 | 0.0280 | LLGL mRNA |
| signal transduction | tigr:HG2709-HT2805 | 100.0 | 0.0156 | Serine/Threonine Kinase |
| signal transduction | L37361 | 100.0 | 0.0365 | ELK receptor tyrosine kinase ligand |
| signal transduction | AF042838 | 100.0 | 0.0469 | MEK kinase 1 (MEKK1) |
| signal transduction | AF068864 | 6.0 | 0.0365 | p21-activated kinase 3 (PAK3) mRNA |
| signal transduction | M73548 | 4.1 | 0.0080 | polyposis locus (DP2.5 gene) mRNA |
| signal transduction | U96919 | 3.6 | 0.0156 | inositol polyphosphate 4-phosphatase type I- |
| | | | | beta mRNA |
| signal transduction | AJ000388 | 3.3 | 0.0113 | calpain-like protease |
| signal transduction | U08023 | 2.3 | 0.0156 | proto-oncogene (c-mer) |
| cell surface glycoprotein | L13283 | 100.0 | 0.0469 | mucin (MG2) |
| cell surface receptor | U33267 | 5.3 | 0.0211 | glycine receptor beta subunit (GLRB) mRNA |
| major histocompatibility | X14975 | 100.0 | 0.0024 | CD1 R2 gene for MHC-related antigen |
| (MHC)-like glycoproteins | | | | |
| membrane-associated protein | X56958 | 100.0 | 0.0113 | ankyrin, Brank-2 protein |
| membrane receptor protein | X98248 | 5.0 | 0.0280 | sortilin |
| membrane-associated protein | U43965 | 3.8 | 0.0156 | ankyrin G119 (ANK3) |
| membrane-associated protein | U13616 | 3.1 | 0.0113 | ankyrin G (ANK-3) mRNA |
| membrane-associated protein | U06452 | 2.4 | 0.0015 | melanoma antigen recognized by T-cells |
| | | | | (MART-1) |
| extracellular matrix/cell cell | X17033 | 100.0 | 0.0469 | integrin alpha-2 subunit |
| contact | | | | |
| extracellular matrix/cell cell | M22092 | 3.1 | 0.0280 | neural cell adhesion molecule (N-CAM) |
| contact | | | | |
| extracellular matrix/cell cell | U68186 | 2.2 | 0.0080 | extracellular matrix protein 1 |
| contact | | | | |
| extracellular matrix/cell cell | J03464 | 2.1 | 0.0469 | collagen alpha-2 type I |
| contact | | | | |
| transcription factor | X82324 | 100.0 | 0.0080 | Brain 4 mRNA |
| transcription factor | X98054 | 100.0 | 0.0009 | G13 protein |
| transcription factor | X59373 | 3.0 | 0.0365 | HOX4D mRNA for a homeobox protein |
| transcription factor | U70862 | 2.5 | 0.0365 | nuclear factor I B3 mRNA |
| channel | L02840 | 4.8 | 0.0280 | potassium channel Kv2.1 mRNA |
| cytoskeleton/cell structure | M94151 | 4.4 | 0.0156 | cadherin-associated protein-related (cap-r) |
| | | | | mRNA |
| cytoskeleton/cell structure | U43959 | 2.2 | 0.0365 | beta 4 adducin |
| 7-transmembrane G-protein | M60284 | 4.4 | 0.0211 | neurokinin A receptor (NK-2R) gene |
| coupledreceptor | | | | |
| serine protease inhibitor | M68516 | 3.7 | 0.0469 | protein C inhibitor gene |
| serine biosynthesis | AF006043 | 2.1 | 0.0037 | 3-phosphoglycerate dehydrogenase |
| Polyadenylation of mRNA | M85085 | 2.0 | 0.0365 | cleavage stimulation factor |
| other | M64936 | 100.0 | 0.0469 | retinoic acid-inducible endogenous retroviral |
| | | | | DNA |
| other | U40992 | 100.0 | 0.0211 | heat shock protein hsp40 homolog |
| other | M25629 | 100.0 | 0.0365 | kallikrein |
| other | U58096 | 100.0 | 0.0365 | testis-specific protein (TSPY) |
| other | AB011406 | 100.0 | 0.0469 | alkaline phosphatase |
| other | U79299 | 4.2 | 0.0211 | neuronal olfactomedin-related ER localized |
| | | | | protein mRNA |
| other | U57911 | 4.2 | 0.0080 | fetal brain (239FB) mRNA, from the WAGR |
| | | | | region |
| other | Y15164 | 3.0 | 0.0280 | cxorf5 (71-7A) gene |
| other | X69392 | 2.7 | 0.0080 | ribosomal protein L26 |
| other | AF003001 | 2.7 | 0.0156 | TTAGGG repeat binding factor 1 (hTRF1-AS) |
| | | | | mRNA |
| other | U39487 | 2.3 | 0.0469 | xanthine dehydrogenaseloxidase |
| other | AF051321 | 2.2 | 0.0211 | Sam68-like phosphotyrosine protein alpha |
| | | | | (SALP) |
| EST/Unknown (N = 28) | | | | |

TABLE 6
Genes Down-Regulated In Women With Endometriosis

| | GeneBank | Fold | | |
|---|---|---|---|---|
| Function/Grouping | ID | Down | p-value | Description (N = 115) |
| calcium-binding protein | Z18948 | 100.0 | 0.0365 | S100E calcium binding protein |
| regulator of vesicular | U44105 | 100.0 | 0.0365 | Rab9 expressed pseudogene |
| transport | | | | |
| RNA polymerase | AF069735 | 100.0 | 0.0365 | PCAF associated factor 65 alpha |
| serine protease | D49742 | 100.0 | 0.0080 | HGF activator like protein |
| serine protease inhibitor | L40377 | 100.0 | 0.0024 | cytoplasmic antiproteinase 2 (CAP2) |
| signal transduction | M64788 | 100.0 | 0.0280 | GTPase activating protein (rap1GAP) |
| signal transduction | L36463 | 100.0 | 0.0365 | ras interactor (RIN1) mRNA |
| signal transduction | L26318 | 100.0 | 0.0469 | protein kinase (JNK1) |
| signal transduction | L13436 | 100.0 | 0.0211 | guanylate cyclase |
| signal transduction | U23852 | 100.0 | 0.0211 | T-lymphocyte specific protein tyrosine kinase |
| | | | | p56lck (lck) abberant mRNA |
| signal transduction | M64322 | 100.0 | 0.0280 | protein tyrosine phosphatase (LPTPase) |
| signal transduction | X75342 | 100.0 | 0.0156 | SHB mRNA |
| signal transduction | X77909 | 6.4 | 0.0211 | IKBL mRNA |
| signal transduction adaptor | U12707 | 2.2 | 0.0469 | Wiskott-Aldrich syndrome protein (WASP) |
| signal transduction//focal | AF023674 | 2.1 | 0.0015 | nephrocystin (NPHP1) |
| adhesion signaling complex | | | | |
| signal transduction/adaptor | AJ223280 | 2.1 | 0.0280 | 36 kDa phosphothyrosine protein |
| protein | | | | |
| transcription factor | AJ001481 | 100.0 | 0.0469 | DUX1 |
| transcription factor | M64673 | 6.4 | 0.0211 | heat shock factor 1 (TCF5) |
| transcription factor/nuclear | M99438 | 2.8 | 0.0113 | transducin-like enhancer protein (TLE3) |
| protein | | | | |
| transcription factor/nuclear | AB006909 | 2.6 | 0.0156 | A-type microphthalmia associated transcription |
| protein | | | | factor |
| transcription factor | U72882 | 2.1 | 0.0024 | interferon-induced leucine zipper protein |
| | | | | (IFP35) |
| transcription factor | S81914 | 2.1 | 0.0365 | IEX-1 = radiation-inducible immediate-early |
| | | | | gene |
| immune function/cytokine— | AJ001383 | 100.0 | 0.0156 | activating NK-receptor (NK-p46) |
| receptor | | | | |
| immune function/cytokine— | Y16645 | 100.0 | 0.0156 | monocyte chemotactic protein-2 |
| receptor | | | | |
| immune function/cytokine— | X67301 | 4.9 | 0.0469 | IgM heavy chain constant region |
| receptor | | | | |
| immune function/cytokine— | AF072099 | 3.7 | 0.0113 | immunoglobulin-like transcript 3 protein variant |
| receptor/immunoreceptor | | | | 1 gene |
| immune function/cytokine— | AF004230 | 3.0 | 0.0113 | monocyte/macrophage Ig-related receptor |
| receptor/immunoreceptor | | | | MIR-7 (MIR cl-7) |
| immune function/cytokine— | M31452 | 3.7 | 0.0037 | proline-rich protein (PRP) |
| receptor/regulation of the | | | | |
| complement system | | | | |
| immune function/cytokine | AF031167 | 2.2 | 0.0211 | interleukin 15 precursor (IL-15) |
| cell surface proteolipid | U17077 | 100.0 | 0.0211 | BENE mRNA |
| cell surface receptor | X61070 | 100.0 | 0.0280 | T cell receptor |
| cell surface receptor | U66497 | 100.0 | 0.0005 | leptin receptor splice variant form 13.2 |
| cell surface adhesion | M25280 | 100.0 | 0.0365 | lymph node homing receptor |
| molecule | | | | |
| cell surface glycoprotein | tigr:HG3477- | 100.0 | 0.0365 | Cd4 Antigen |
| | HT3670 | | | |
| cell surface glycoprotein | X17033 | 100.0 | 0.0469 | integrin alpha-2 subunit |
| cell surface glycoprotein | tigr:HG3175- | 4.3 | 0.0056 | Carcinoembryonic Antigen |
| | HT3352 | | | |
| cell surface receptor | M31932 | 3.3 | 0.0280 | IgG low affinity Fc fragment receptor (FcRIIa) |
| cell surface receptor | D13168 | 2.6 | 0.0280 | endothelin-B receptor (hET-BR) |
| cell surface glycoprotein | M5991 | 12.6 | 0.0280 | integrin alpha-3 chain |
| cytoskeleton/cell structure | J03796 | 100.0 | 0.0469 | erythroid isoform protein 4.1 mRNA |
| cytoskeleton/cell | U53204 | 3.6 | 0.0365 | plectin (PLEC1) |
| structure/intermediate | | | | |
| filament binding protein | | | | |
| carrier for retinol | X00129 | 12.6 | 0.0080 | retinol binding protein (RBP) |
| apoptosis related | D38122 | 9.1 | 0.0469 | Fas ligand |
| ion transport regulators or | U28249 | 8.4 | 0.0024 | 11kd protein |
| channels | | | | |
| 7-transmembrane G-protein | AF095448 | 6.5 | 0.0113 | G protein-coupled receptor (RAIG1) |
| coupled receptor | | | | |
| 7-transmembrane G-protein | AF056085 | 2.2 | 0.0365 | GABA-B receptor mRNA |
| coupled receptor | | | | |
| secretory protein | V00511 | 6.2 | 0.0024 | pregastrin |
| secretory protein | M63193 | 4.7 | 0.0024 | platelet-derived endothelial cell growth factor |
| secretory protein | M31682 | 4.0 | 0.0365 | inhibin beta-B-subunit |
| secretory protein | U29195 | 3.3 | 0.0024 | neuronal pentraxin II (NPTX2) |
| secretory protein | M57730 | 2.9 | 0.0015 | B61 mRNA |
| secretory protein | AB020315 | 2.9 | 0.0365 | Dickkopf-1 (hdkk-1) |
| secretory protein/growth | AF055008 | 2.6 | 0.0365 | epithelin 1 and 2 |
| factor | | | | |
| secretory protein | M61886 | 2.5 | 0.0280 | pregnancy-associated endometrial alpha2- |
| | | | | globulin |
| secretory protein | J04129 | 2.2 | 0.0365 | Human placental protein 14 (PP14 |
| vitamin B12-binding protein | J05068 | 5.7 | 0.0005 | transcobalamin I |
| extracellular matrix/cell cell | Z15008 | 5.6 | 0.0113 | laminin |
| contact | | | | |
| extracellular matrix protein | X82494 | 2.2 | 0.0469 | fibulin-2 |
| extracellular matrix protein | X15998 | 2.2 | 0.0113 | chondroitin sulphate proteoglycan versican, V1 |
| | | | | splice-variant |
| extracellular matrix/cell cell | X15606 | 2.1 | 0.0080 | ICAM-2, cell adhesion ligand for LFA-1 |
| contact | | | | |
| organic cation transporter | AB007448 | 5.2 | 0.0024 | OCTN1 |
| membrane-associated protein | U04343 | 3.8 | 0.0113 | CD86 antigen |
| membrane protein/tight | U89916 | 2.3 | 0.0469 | claudin-10 (CLDN10) |
| junction | | | | |
| transporter | U08989 | 3.1 | 0.0280 | glutamate transporter |
| transporter | U21936 | 2.9 | 0.0015 | Human peptide transporter (HPEPT1) |
| plasma | | | | |
| metalloprotein/peroxidation of | M13699 | 3.0 | 0.0080 | ceruloplasmin (ferroxidase) |
| Fe(II) transferrin to form | | | | |
| Fe(III) transferrin/essential for | | | | |
| iron homeostasis | | | | |
| oncogene/protein kinase | M16750 | 2.3 | 0.0156 | pim-1 oncogene |
| tumor suppressor | X92814 | 2.2 | 0.0211 | HREV107-like protein |
| tumor suppressor | AB012162 | 2.0 | 0.0024 | APCL protein |
| cell cycle | M69199 | 2.3 | 0.0280 | G0S2 protein |
| cell cycle/gatekeeper in DNA | AF076838 | 2.3 | 0.0080 | Rad17-like protein (RAD17) |
| damage checkpoint control | | | | |
| other | AB020735 | 100.0 | 0.0113 | ENDOGL-2 endonuclease G-like protein-2 |
| other | D84454 | 100.0 | 0.0009 | UDP-galactose translocator |
| other | M38180 | 100.0 | 0.0280 | 3-beta-hydroxysteroid dehydrogenase/delta-5- |
| | | | | delta-4-isomerase (3-beta-HSD) |
| other | Y11731 | 100.0 | 0.0056 | DNA glycosylase |
| other | AF007170 | 100.0 | 0.0280 | DEME-6 mRNA |
| other | AB014679 | 4.7 | 0.0037 | N-acetylglucosamine-6-O-sulfotransferase |
| | | | | (GlcNAc6ST) |
| other/urea cycle | K02100 | 4.0 | 0.0113 | ornithine transcarbamylase (OTC) |
| other | M25079 | 3.6 | 0.0211 | beta-globin |
| other/synthesis of cytochrome | AL021683 | 3.5 | 0.0469 | homologous to Yeast SCO1 & SCO2 |
| C oxidase | | | | |
| other/catalyzes the transfer of | AB017566 | 2.9 | 0.0365 | lipoyltransferase |
| the lipoyl group | | | | |
| other/anchoring of cell surface | AF022913 | 2.6 | 0.0211 | GPI transamidase |
| proteins | | | | |
| other/phosphorolytic cleavage | X00737 | 2.2 | 0.0080 | purine nucleoside phosphorylase |
| of inosine to hypoxanthine | | | | |
| other/regulator of vitamin A | AF061741 | 2.2 | 0.0469 | retinal short-chain dehydrogenase/reductase |
| metabolism | | | | retSDR1 mRNA |
| other/intramitochondrial free | X07834 | 2.1 | 0.0211 | manganese superoxide dismutase |
| radical scavenging enzyme | | | | |
| other | AF093420 | 2.0 | 0.0365 | Hsp70 binding protein HspBP1 |
| EST/Unknown (N = 29) | | | | |

Clustering

Stringent data filtering permits identification of significantly and consistently changed genes. Clustering further allows grouping of genes of biological relevance in eutopic endometrium during the window of implantation of women with endometriosis. We performed unsupervised cluster analysis, based on NCBI (National Center for Biotechnology Information)/Entrez/OMIM (Online Mendelian Inheritance in Man) database searches, which segregated genes of interest into various categories (Tables 5 & 6). The most highly up-regulated genes, reaching the upper limit of the program algorithm of 100 fold, include those involved in: apoptosis [cysteine protease (ICErel-III)], protein or RNA processing [putative mono-ADP-ribosyltransferase] [RNA-binding protein CUG-BP], transporter protein [bile salt export pump (BSEP)], zinc metalloenzyme [carbonic anhydrase I (CAI)], DNA repair [PMS7 mRNA (yeast mismatch repair gene PMS1 homologue), DNA primase], immune function [alpha interferon, transforming growth factor beta-3], secretory protein [secretogranin II], signal transduction [Serine/Threonine Kinase, ELK receptor tyrosine kinase ligand, MEK kinase 1], cell surface protein [mucin, MHC-related antigen] and transcription factors [Brain 4, G13]. Other genes of unspecified biological pathways such as retinoic acid-inducible endogenous retroviral DNA, heat shock protein hsp40 homolog, kallikrein, testis-specific protein (TSPY) and alkaline phosphatase also were up-regulated to the algorithm maximum of 100-fold. Other up-regulated genes include members of cytokine receptor families, secretory proteins, signal transduction, cell surface receptors, membrane-associated proteins and extracellular matrix/cell-cell contact, potassium channel, cytoskeleton/cell structure, neurokinin receptor, and others.

The most highly down-regulated genes include those involved in: calcium-binding [S100E calcium binding protein], regulator of vesicular transport [Rab9 expressed pseudogene], RNA polymerase [PCAF associated factor 65 alpha], serine protease/inhibitor [HGF activator like protein, cytoplasmic antiproteinase 2 (CAP2)], signal transduction [GTPase activating protein (rap1GAP), ras interactor (RIN1), protein kinase JNK1, protein tyrosine phosphatase (LPTPase)], transcription factor [DUX1], immune function [activating NK-receptor (NK-p46), monocyte chemotactic protein-2], cell surface proteins [T cell receptor, leptin receptor splice variant, integrin alpha-2 subunit], all reached 100-fold difference, as did ENDOGL-2 endonuclease G-like protein-2 and DNA glycosylase. Down-regulated genes also included signal transduction, immune function and cytokine/receptor genes, cell surface glycoproteins/receptors, retinol binding protein, ion transporters, secretory proteins including inhibin beta-B, B61, Dickkopf-1, and glycodelin, GlcNAc6ST/GlcNAC, and others.

Validation of Gene Expression

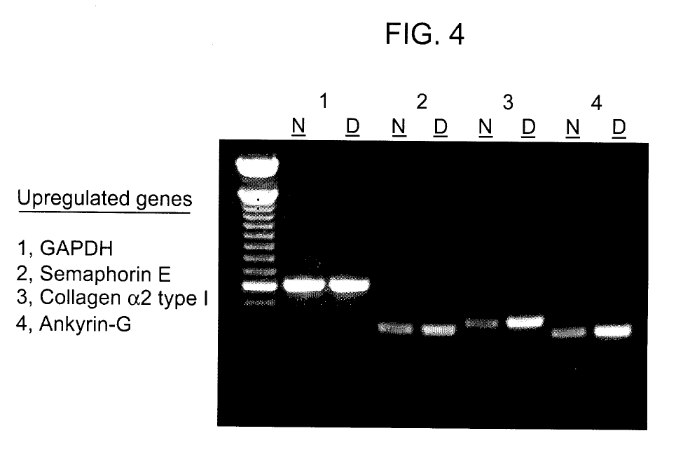

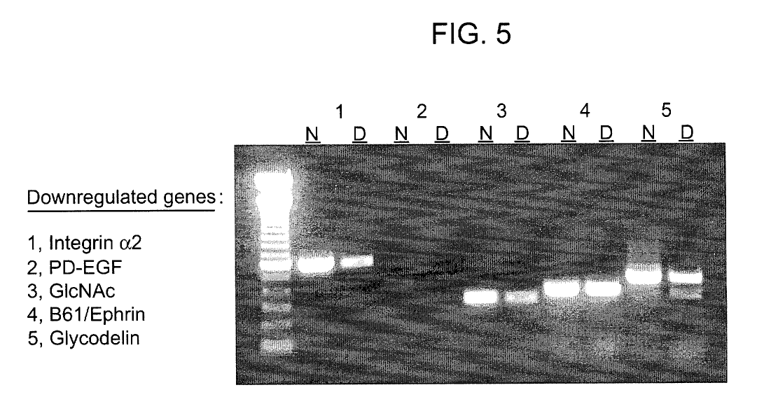

Expression of select up-regulated and down-regulated genes was validated by RT-PCR and/or Northern or dot blot analysis, using RNA isolated from endometrial biopsy samples in the window of implantation, from women with and without endometriosis. The results are shown in FIGS. 4-6. For the RT-PCR studies, the primer sets are shown in Table 4. Although real-time quantitative PCR was not performed, the RT-PCR data in FIGS. 4 and 5 demonstrate clearly in the women with endometriosis compared to normal fertile women, up-regulation of semaphorin E, collagen α₂ type 1, ankyrin G, and down-regulation of integrin □₂, platelet-derived endothelial cell growth factor (PD-EGF), N-actelyglucosamine-6-O-sulfotransferase (GlcNAc6ST/GlcNAC), B61/ephrin, and glycodelin. These data are consistent with the observations from the microarray data (Tables 5 and 6).

FIG. 4: Equal cycle RT-PCR of selected genes up-regulated in eutopic human endometrium during the window of implantation, from women without (N) and with (D) endometriosis. Specific primer pairs used are shown in Table 4. Appropriate size bands are depicted for the housekeeping gene GAPDH (lane 1), as well as for the upregulated genes: semaphorin E (lane 2), collagen alpha-2 type I (lane 3) and ankyrin G (lane 4). Two samples from women without and two from women with endometriosis were used for this study; representative results are shown.

FIG. 5: Equal cycle RT-PCR of selected genes down-regulated in eutopic human endometrium during the window of implantation, from women without (N) and with (D) endometriosis. Specific primer pairs used are shown in Table 4. Appropriate size bands are depicted for: integrin alpha2 subunit (lanes 1), PD-EGF (lanes 2), GlcNAc (lanes 3), B61/Ephrin (lanes 4) and Glycodelin (lanes 5). Two samples from women without and two from women with endometriosis were used for this study; representative results are shown.

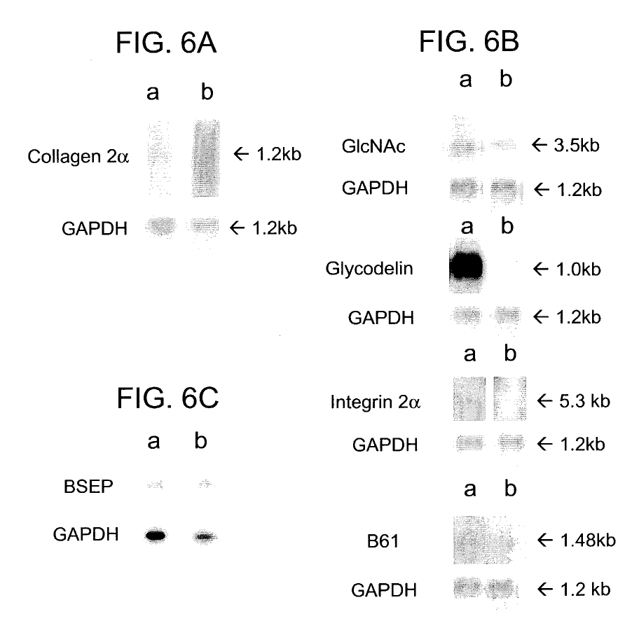

Northern or dot-blot analyses were also conducted to validate changes in gene expression in the samples from women with endometriosis versus normal controls. Representative Northern blots and dot-blots are demonstrated in FIG. 6. Densitometric analyses of band intensities and dot intensities were conducted, and GAPDH was used as a control to determine relative mRNA expression in each sample. Normalized relative expressions of select mRNAs in endometrium during the implantation window in women with versus without endometriosis were then calculated. The data demonstrate up-regulation of collagen a type I of 2.63-fold, bile salt export pump (BSEP) of 1.97-fold; and down-regulation of GlcNAC, 1.75-fold; glycodelin, 51.5-fold; integrin α2, 1.82-fold; and B61/ephrin, 4.46-fold in endometrium from women with versus without endometriosis. Northern and dot blot analyses parallel results obtained from the microarray expression profiling analysis, although exact fold-change differences are not the same as in the microarray analysis (Tables 5 and 6). The fold changes are not necessarily identical among various methodologies due to several factors such as tissue heterogeneity, subject-to-subject biologic variation and the lower abundance of specific mRNAs relative to the highly abundant GAPDH mRNA.

FIGS. 6A-C Northern blot analyses demonstrating: (A) up-regulation of collagen alpha-2 type 1, (B) down-regulation of GlcNAc, glycodelin, integrin 2 α subunit and B61, in eutopic human endometrium during the window of implantation, from women without (a) or with (b) endometriosis. FIG. 6C demonstrates dot-blot analysis for up-regulation of BSEP. Three samples from women without and two from women with endometriosis were used and representative results are shown. GAPDH hybridization densities of corresponding lanes are also shown for comparison, and subsequent densitometric calculations.

Target Identification

Comparisons were made between differentially expressed genes in the implantation window in endometrium of women with versus without endometriosis and genes previously identified to be differentially up- or down-regulated in normal human endometrium in the implantation window compared to the late proliferative phase (Kao et al., supra). Twelve target genes of three distinct patterns are identified. In group 1, eight genes normally up-regulated in the window of implantation were significantly down-regulated in endometrium of women with endometriosis. In group 2, genes normally down-regulated during the window of implantation, three were up regulated in endometriosis. In group 3, one gene already down regulated in the normal window of implantation was further down-regulated with endometriosis. Group 1 consists of IL-15, proline-rich protein, B61, Dickkopf-1, glycodelin, N-acetylglucosamine-6-O-sulfotransferase (GlcNAc6ST), G0S2 protein and purine nucleoside phosphorylase. Group 2 consists of semaphorin E, neuronal olfactomedin-related ER localized protein mRNA, and Sam68-like phosphotyrosine protein alpha (SALP), and Group 3 is represented by a single gene, neuronal pentraxin II (NPTX2).

What is claimed is:

1. A method of detecting, in a biological sample, a gene product that a gene product that is differentially expressed in the endometrium during the window of implantation, the method comprising contacting the biological sample with a binding agent specific for the gene product.

2. The method of claim 1, wherein the gene product is an mRNA that is normally upregulated during the window of implantation and that is down-regulated in endometriosis.

3. The method of claim 2, wherein the gene product is a protein encoded by the mRNA.

4. The method of claim 1, wherein the gene product is an mRNA that is normally down-regulated during the window of implantation and that is up-regulated in endometriosis.

5. The method of claim 4, wherein the gene product is a protein encoded by the mRNA.

6. A method for the diagnosis of endometrial disorders, the method comprising:
determining the upregulation of expression in any one of the sequences set forth in Table 5.

7. The method according to claim 6, wherein said determining comprises detecting the presence of increased amounts of mRNA or polypeptide in endometrial cells.

8. A method for the diagnosis of endometrial disorders, the method comprising:
determining the downregulation of expression in any one of the sequences set forth in Table 6.

9. The method according to claim 8, wherein said determining comprises detecting the presence of increased amounts of mRNA or polypeptide in endometrial cells.

10. An array of nucleic acids, comprising:
two or more nucleic acids comprising sequences set forth in Table 2, Table 3, Table 5, and Table 6.

11. A method for determining the probability of success of blastocyst implantation following an assisted reproductive technology or naturally achieved conception, the method comprising:
determining the level, in a biological sample, of a gene product that is differentially expressed in the endometrium during the window of implantation;
comparing the level to a standard; and
determining the probability of success of implantation following an assisted reproductive technology or naturally achieved conception based on the level of the gene product.

12. The method of claim 11, wherein the gene product is an mRNA that is normally upregulated during the window of implantation.

13. The method of claim 12, wherein the gene product is a protein encoded by the mRNA.

14. The method of claim 11, wherein the gene product is an mRNA that is normally down-regulated during the window of implantation.

15. The method of claim 14, wherein the gene produce is a protein encoded by the mRNA.

16. A kit for detecting a level, in a biological sample, of a gene product that is differentially expressed in the endometrium during the window of implantation, the kit comprising a detectably labeled binding agent that binds specifically to the gene product.

17. The kit according to claim 16, wherein the kit further comprises an unlabeled binding agent that binds specifically to the gene product, wherein the unlabeled binding agent is bound to an insoluble support.

18. The kit according to claim 16, wherein the binding agent is an antibody.

19. The kit according to claim 16, wherein the binding agent is a nucleic acid.

20. A method of identifying an agent that modulates a level of a gene product that is differentially expressed in the window of implantation, the method comprising:
contacting a test agent in vitro with a eukaryotic cell that produces a gene product that is differentially expressed in the window of implantation; and
determining the effect, if any, on the level of the gene product.

21. The method of claim 20, wherein the agent increases the level of the gene product.

22. The method of claim 20, wherein the agent decreases the level of the gene product.


SEQUENCE LISTING (42 sequences)

SEQ ID NO: 1 - DNA, 19 bp, Artificial Sequence
  primer
actctgctggtgcgtctac

SEQ ID NO: 2 - DNA, 20 bp, Artificial Sequence
  primer
ttaaccgtcctccttcaaac

SEQ ID NO: 3 - DNA, 23 bp, Artificial Sequence
  primer
aagttggcagggacctggcactc

SEQ ID NO: 4 - DNA, 23 bp, Artificial Sequence
  primer
acggcacggctcttccatctgtt

SEQ ID NO: 5 - DNA, 19 bp, Artificial Sequence
  primer
tactccgccaagtattctg

SEQ ID NO: 6 - DNA, 22 bp, Artificial Sequence
  primer
attacagtgatgaatagctctt

SEQ ID NO: 7 - DNA, 20 bp, Artificial Sequence
  primer
aggcgtgcaaatctgtctcg

SEQ ID NO: 8 - DNA, 23 bp, Artificial Sequence
  primer
tgcatttggatagctggtttagt

SEQ ID NO: 9 - DNA, 21 bp, Artificial Sequence
  primer
gctggggctatgatggaaatg

SEQ ID NO: 10 - DNA, 23 bp, Artificial Sequence
  primer
ctagcaaggccccaaacacaaag

SEQ ID NO: 11 - DNA, 20 bp, Artificial Sequence
  primer
agttgctgatggtcctcatg

SEQ ID NO: 12 - DNA, 19 bp, Artificial Sequence
  primer
agaaggtgtggtttgcagc

SEQ ID NO: 13 - DNA, 19 bp, Artificial Sequence
  primer
aaaagctccaggtcccttc

SEQ ID NO: 14 - DNA, 20 bp, Artificial Sequence
  primer
agggtttcttgccaagatcc

SEQ ID NO: 15 - DNA, 20 bp, Artificial Sequence
  primer
cttcctgctctacaagatcg

SEQ ID NO: 16 - DNA, 20 bp, Artificial Sequence
  primer
cctcatctgagtacacagtg

SEQ ID NO: 17 - DNA, 24 bp, Artificial Sequence
  primer
ccgtgctgcgcttcttcttctgtg

SEQ ID NO: 18 - DNA, 24 bp, Artificial Sequence
  primer
gcgggacttgagttcgagggatgg

SEQ ID NO: 19 - DNA, 20 bp, Artificial Sequence
  primer
ctctcaataggaaagagaag

SEQ ID NO: 20 - DNA, 20 bp, Artificial Sequence
  primer
tgaataagacacagtcacac

SEQ ID NO: 21 - DNA, 19 bp, Artificial Sequence
  primer
ttgctgtcctccagctctg

SEQ ID NO: 22 - DNA, 18 bp, Artificial Sequence
  primer
caggctccagatatgaac

SEQ ID NO: 23 - DNA, 21 bp, Artificial Sequence
  primer
cacagtccatgccatcactgc

SEQ ID NO: 24 - DNA, 21 bp, Artificial Sequence
  primer
ggtctacatggcaactgtgag

SEQ ID NO: 25 - DNA, 20 bp, Artificial Sequence
  primer
ctgatgggagataccatgtc

SEQ ID NO: 26 - DNA, 20 bp, Artificial Sequence
  primer
tcctctgcattgagtcagtg

SEQ ID NO: 27 - DNA, 20 bp, Artificial Sequence
  primer
tgcaccacttgtggcttttg

SEQ ID NO: 28 - DNA, 20 bp, Artificial Sequence
  primer
aagcttctgtggaaccatgg

SEQ ID NO: 29 - DNA, 20 bp, Artificial Sequence
  primer
aatgcttgccgctttagagg

SEQ ID NO: 30 - DNA, 20 bp, Artificial Sequence
  primer
atcgactaggtcatccagtg

SEQ ID NO: 31 - DNA, 20 bp, Artificial Sequence
  primer
aatgtcaagtggcagctcag

SEQ ID NO: 32 - DNA, 20 bp, Artificial Sequence
  primer
ccctatccttagccttagag

SEQ ID NO: 33 - DNA, 20 bp, Artificial Sequence
  primer
ctcttcggatgggaatgttc

SEQ ID NO: 34 - DNA, 20 bp, Artificial Sequence
  primer
ttgcaaccagagctaacagc

SEQ ID NO: 35 - DNA, 20 bp, Artificial Sequence
  primer
atggatctggaggagacctc

SEQ ID NO: 36 - DNA, 20 bp, Artificial Sequence
  primer
agaatggaggctgtgatgag

SEQ ID NO: 37 - DNA, 20 bp, Artificial Sequence
  primer
tgattccctgtggtgatacc

SEQ ID NO: 38 - DNA, 20 bp, Artificial Sequence
  primer
cccacttcaaaatggaaggc

SEQ ID NO: 39 - DNA, 20 bp, Artificial Sequence
  primer
aaacaagctgtgcaggcatg

SEQ ID NO: 40 - DNA, 20 bp, Artificial Sequence
  primer
ccttacagctacactctaag

SEQ ID NO: 41 - DNA, 23 bp, Artificial Sequence
  primer
aagttggcagggacctggcactc

SEQ ID NO: 42 - DNA, 23 bp, Artificial Sequence
  primer
acggcacggctcttccatctgtt